SEQUENCE LISTING (13 sequences)

SEQ ID NO: 1 - protein, 591 aa, Caulobacter crescentus
MSNRTPRRFRSRDWFDNPDHIDMTALYLERFMNYGITPEELRSGKPIIGIAQTGSDISPCNRIHLDLVQRVRDGIRDAGGIPMEFPVHPIFENCRRPTAALDRNLSYLGLVETLHGYPIDAVVLTTGCDKTTPAGIMAATTVNIPAIVLSGGPMLDGWHENELVGSGTVIWRSRRKLAAGEITEEEFIDRAASSAPSAGHCNTMGTASTMNAVAEALGLSLTGCAAIPAPYRERGQMAYKTGQRIVDLAYDDVKPLDILTKQAFENAIALVAAAGGSTNAQPHIVAMARHAGVEITADDWRAAYDIPLIVNMQPAGKYLGERFHRAGGAPAVLWELLQQGRLHGDVLTVTGKTMSENLQGRETSDREVIFPYHEPLAEKAGFLVLKGNLFDFAIMKSSVIGEEFRKRYLSQPGQEGVFEARAIVFDGSDDYHKRINDPALEIDERCILVIRGAGPIGWPGSAEVVNMQPPDHLLKKGIMSLPTLGDGRQSGTADSPSILNASPESAIGGGLSWLRTGDTIRIDLNTGRCDALVDEATIAARKQDGIPAVPATMTPWQEIYRAHASQLDTGGVLEFAVKYQDLAAKLPRHNH

SEQ ID NO: 2 - protein, 338 aa, Escherichia coli
MRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPD

SEQ ID NO: 3 - protein, 348 aa, Escherichia coli
MTQFNPVDHPHRRYNPLTGQWILVSPHRAKRPWQGAQETPAKQVLPAHDPDCFLCAGNVRVTGDKNPDYTGTYVFTNDFAALMSDTPDAPESHDPLMRCQSARGTSRVICFSPDHSKTLPELSVAALTEIVKTWQEQTAELGKTYPWVQVFENKGAAMGCSNPHPHGQIWANSFLPNEAEREDRLQKEYFAEQKSPMLVDYVQRELADGSRTVVETEHWLAVVPYWAAWPFETLLLPKAHVLRITDLTDAQRSDLALALKKLTSRYDNLFQCSFPYSMGWHGAPFNGEENQHWQLHAHFYPPLLRSATVRKFMVGYEMLAETQRDLTAEQAAERLRAVSDIHFRESGV

SEQ ID NO: 4 - protein, 382 aa, Escherichia coli
MSLKEKTQSLFANAFGYPATHTIQAPGRVNLIGEHTDYNDGFVLPCAIDYQTVISCAPRDDRKVRVMAADYENQLDEFSLDAPIVAHENYQWANYVRGVVKHLQLRNNSFGGVDMVISGNVPQGAGLSSSASLEVAVGTVLQQLYHLPLDGAQIALNGQEAENQFVGCNCGIMDQLISALGKKDHALLIDCRSLGTKAVSMPKGVAVVIINSNFKRTLVGSEYNTRREQCETGARFFQQPALRDVTIEEFNAVAHELDPIVAKRVRHILTENARTVEAASALEQGDLKRMGELMAESHASMRDDFEITVPQIDTLVEIVKAVIGDKGGVRMTGGGFGGCIVALIPEELVPAVQQAVAEQYEAKTGIKETFYVCKPSQGAGQC

SEQ ID NO: 5 - protein, 346 aa, Escherichia coli
MLNETPALAPDGQPYRLLTLRNNAGMVVTLMDWGATLLSARIPLSDGSVREALLGCASPECYQDQAAFLGASIGRYANRIANSRYTFDGETVTLSPSQGVNQLHGGPEGFDKRRWQIVNQNDRQVLFALSSDDGDQGFPGNLGATVQYRLTDDNRISITYRATVDKPCPVNMTNHVYFNLDGEQSDVRNHKLQILADEYLPVDEGGIPHDGLKSVAGTSFDFRSAKIIASEFLADDDQRKVKGYDHAFLLQAKGDGKKVAAHVWSADEKLQLKVYTTAPALQFYSGNFLGGTPSRGTEPYADWQGLALESEFLPDSPNHPEWPQPDCFLRPGEEYSSLTEYQFIAE

SEQ ID NO: 6 - DNA, 1922 bp, Artificial Sequence
  xylD expression cassette
aggctgtctcgtctcgtctctttacggctagctcagtcctaggtacaatgctagcaacaacagcttagaaggaggtcaatatgaggtccgccttgtctaaccgcacgccccgccggttccggtcccgcgattggttcgataaccccgaccatatcgacatgaccgcgctctatctggagcgcttcatgaactacgggatcacgccggaggagctgcgcagcggcaagccgatcatcggcatcgcccagaccggcagcgacatctcgccctgcaaccgcatccacctggacctggtccagcgggtgcgggacgggatccgcgacgccgggggcatccccatggagttcccggtccatccgatcttcgagaactgccgtcgcccgacggcggcgctggaccggaacctctcgtacctgggtctcgtcgagaccctgcacggctatccgatcgacgccgtggttctgaccaccggctgcgacaagaccaccccggccgggatcatggccgccaccacggtcaatatcccggccatcgtgctgtcgggcggcccgatgctggacggctggcacgagaacgagctcgtgggctcgggcaccgtgatctggcgctcgcgccgcaagctggcggccggcgagatcaccgaggaagagttcatcgaccgcgccgccagctcggcgccgtcggcgggccactgcaacaccatgggcacggcctcgaccatgaacgccgtggccgaggcgctgggcctgtcgctgaccggctgcgcggccatccccgccccctaccgcgagcgcggccagatggcctacaagaccggccagcgcatcgtcgatctggcctatgacgacgtcaaaccgctcgacatcctgaccaagcaagccttcgagaacgccatcgccctggtggcggcggccggcggctcgaccaacgcccagccgcacatcgtggccatggcccgtcacgccggcgtcgagatcaccgccgacgactggcgcgcggcctatgacatcccgctgatcgtcaacatgcagccggccggcaagtatctgggcgagcgcttccaccgagccggcggcgcgccggcggtgctgtgggagctgttgcagcaaggccgcctgcacggcgacgtgctgaccgtcaccggcaagacgatgagcgagaacctgcaaggccgcgaaaccagcgaccgcgaggtgatcttcccgtaccacgagccgctggccgagaaggccgggttcctggttctcaagggcaacctcttcgacttcgcgatcatgaagtccagcgtgatcggcgaggagttccgcaagcgctacctgtcgcagcccggccaggaaggcgtgttcgaagcccgcgccatcgtgttcgacggctcggacgactatcacaagcggatcaacgatccggccctggagatcgacgagcgctgcatcctggtgatccgcggcgcgggtccgatcggctggcccggctcggccgaggtcgtcaacatgcagccgccggatcaccttctgaagaaggggatcatgagcctgcccaccctgggcgatggccgtcagtcgggcaccgccgacagcccctcgatcctgaacgcctcgcccgaaagcgcgatcggcggcggcctgtcgtggctgcgcaccggcgacaccatccgcatcgacctcaacaccggccgctgcgacgccctggtcgacgaggcgacgatcgccgcgcgcaagcaggacggcatcccggcggttcccgccaccatgacgccctggcaggaaatctaccgcgcccacgccagtcagctcgacaccggcggcgtgctggagttcgcggtcaagtaccaggacctggcggccaagctgccccgccacaaccactgagctgggagttcgtagacggacgcaaaaaaccccgcttcggcggggttttttcgc

SEQ ID NO: 7 - DNA, 15 bp, Artificial Sequence
  Insertion sequence
gcgccagttcgtggc

SEQ ID NO: 8 - DNA, 12 bp, Artificial Sequence
  Insertion sequence
ctgagcctggcc

SEQ ID NO: 9 - DNA, 35 bp, Artificial Sequence
  BBa_J23110 promoter
tttacggctagctcagtcctaggtacaatgctagc

SEQ ID NO: 10 - DNA, 25 bp, Artificial Sequence
  5′-untranslated region
aacaacagcttagaaggaggtcaat

SEQ ID NO: 11 - DNA, 20 bp, Artificial Sequence
  Insulating sequence
aggctgtctcgtctcgtctc

SEQ ID NO: 12 - DNA, 20 bp, Artificial Sequence
  Insulating sequence
gctgggagttcgtagacgga

SEQ ID NO: 13 - DNA, 34 bp, Artificial Sequence
  BBa_B1002 terminator
cgcaaaaaaccccgcttcggcggggttttttcgc

(19) United States
(12) Patent Application Publication
(10) Pub. No.: US 2023/0119263 A1
(43) Pub. Date: Apr. 20, 2023

(54) Pseudomonas mutant strains with enhanced xylose and galactose utilization

(71) Applicant: The Regents of the University of California, Oakland, CA (US)

(72) Inventors: Hyungyu Lim, San Diego, CA (US); Thomas T. Eng, El Cerrito, CA (US); Adam M. Feist, San Diego, CA (US)

(21) Appl. No.: 17/709,332

(22) Filed: Mar. 30, 2022

Related U.S. Application Data
(60) Provisional application No. 63/168,687, filed on Mar. 31, 2021.
(60) Provisional application No. 63/217,759, filed on Jul. 1, 2021.

(51) Int. Cl. C12N 1/20; C12N 15/52; C12N 9/88; C12N 9/90; C12N 9/12

(52) U.S. Cl. CPC C12N 1/20; C12N 15/52; C12N 9/88; C12Y 402/01082; C12N 9/90; C12Y 501/03002; C12N 9/1241; C12Y 207/07012; C12N 9/1205; C12Y 207/01006; C12R 2001/40

(57) ABSTRACT

The present invention provides for a Pseudomonas cell is able to grow in a medium with xylose or galactose as a sole carbon source with a growth rate of equal to or higher than 0.10 h⁻¹. The present invention provides for methods and compositions relating to an engineered Pseudomonas putida KT2440 utilizing a non-native carbon source, such as galactose or xylose or both.

### CROSS-REFERENCE TO RELATED APPLICATIONS

This application claims priority to U.S. Provisional Patent Application Ser. Nos. 63/168,687, filed on Mar. 31, 2021, and 63/217,759, filed on Jul. 1, 2021, both of which are hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of Pseudomonas mutant strains.

### BACKGROUND OF THE INVENTION

Pseudomonas putida KT2440 (hereafter, KT2440) has been widely studied for its utilization as a microbial platform in biorefinery processes due to its tolerance to various stresses and the ability to grow on biomass-abundant aromatics (e.g., coumaric acid) (Belda et al., 2016; Nikel and de Lorenzo, 2018; Nogales et al., 2020). Because conventional microbial hosts such as Escherichia coli are incapable of utilizing such aromatics, the use of KT2440 is expected to improve cost effectiveness of bioprocesses by achieving whole-conversion of biomass-derivable carbon sources. In this regard, so far, various studies have been conducted to produce various biochemicals using KT2440 (Bentley et al., 2020; Nikel and de Lorenzo, 2018).

Despite these great advantages, one drawback in the use of KT2440 is that it cannot metabolize several sugars (e.g., xylose, galactose) obtainable from biomass (Isikgor and Becer, 2015; Lim et al., 2013). While few Pseudomonas species are known to naturally have enzymes to catabolize xylose and galactose (Buckel and Zehelein, 1981; Kohler et al., 2015; Liu et al., 2015), KT2440 lacks the utilization pathway of these sugars, requiring its engineering for their efficient conversion into biochemicals. To enable xylose and galactose utilization, KT2440 has previously been engineered by the heterologous introduction of key missing genes in known sugar utilization pathways (Table 2 and FIG. 6). Specifically, the xylose isomerase pathway has been constructed by the expression of xylose isomerase (XylA) and xylulokinase (XylB) from E. coli (Dvořák and de Lorenzo, 2018; Le Meur et al., 2012; Meijnen et al., 2008; Y. Wang et al., 2019). Additionally, the Weimberg pathway (i.e., xylose oxidative pathway) has been constructed by the expression of xylonate dehydratase (XylD) from Caulobacter crescentus (Bator et al., 2019; Meijnen et al., 2009; Shen et al., 2020). Enabling galactose utilization has been less studied compared to xylose metabolism; two studies reported the construction of De Ley-Doudoroff (DLD) pathway (i.e., galactose oxidative pathway) (Peabody et al., 2019) or Leloir pathway (Banerjee et al., 2020) by the expression of DgoKAD, galactonate catabolic enzymes, from P. fluorescens SBW2 or GalETKM, galactose operon, from E. coli K-12 MG1655, respectively.

TABLE 2
Previous studies for enabling non-native sugars in P. putida strains.

| | | | | | | Biomass | |
|---|---|---|---|---|---|---|---|
| | | | | Growth | Sugar | yield | |
| | | | | rate | uptake rate | (g DCW | |
| Sugar | Pathway | Expression | Approach | (h⁻¹) | (g L⁻¹ h⁻¹) | g⁻¹ sugar) | Reference |
| Xylose | Isomerase | Plasmid | Host: P. putida S12 | 0.35 | — | 0.52 | (Meijnen et al.. |
| | pathway | | Heterologous expression of xylAB | | | | 2008) |
| | | | from Eschertchia coli | | | | |
| | | | Adaptive laboratory evolution in | | | | |
| | | | a xylose medium | | | | |
| Xylose | Isomerase | Plasmid | Host: P. putida KT2440 | 0.24 | 2.5 | 0.5 | (Le Meur et al., |
| | pathway | | Heterologous expression of xylAB | | | | 2008) |
| | | | from E. coli | | | | |
| Xylose | Isomerase | Plasmid | Host: P. putida EM42 | 0.17 | 0.05 | 0.27 | (Dvo\check{r}ák and de |
| | pathway | | (KT2440 derivative) | | | | Lorenzo, 2018) |
| | | | Heterologous expression of xylAB | | | | |
| | | | from E. coli | | | | |
| Xylose | Isomerase | Plasmid | Host: P. putida KT2440 | 0.39 | 0.72 | 0.30 | (Wang et al., |
| | pathway | | Heterologous expression of | | | | 2019) |
| | | | xylAB from E. coli | | | | |
| Xylose | Isomerase | Plasmid | Host: P. putida KT2440 | 0.02 | 0.07 | — | (Bator et al., |
| | pathway | | Heterologous expression of xylAB | | | | 2019) |
| | | | from E. coli | | | | |
| Xylose | Isomerase | Chromosome | Host: P. putida KT2440 | 0.32 | — | — | (Elmore et al., |
| | pathway | | Heterologous expression of xylAB, | | | | 2020) |
| | | | [symbol] , [symbol] xylE from E. coli | | | | |
| | | | Adaptive laboratory evolution in | | | | |
| | | | xylose medium | | | | |
| Xylose | Weimberg | Plasmid | Host: P. putida S12 | 0.21 | — | 0.53 | (Meijneu et al., |
| | pathway | | Heterologous expression of xylXABCD | | | | 2009) |
| | | | from Caulobacter crescentus | | | | |
| Xylose | Weimberg | Plasmid | Host: P. putida KT2440 | 0.21-0.3 [symbol] | 0.29-0.45 | — | (Bator et al., |
| | pathway | | Heterologous expression of PVLB18550, | | | | 2019) |
| | | | PVLB18555, PVLB18560, PVLB18565 from | | | | |
| | | | P. [symbol] VLB120 | | | | |
| Xylose | Weimberg | Chromosome | Host: P. putida KT2440 | 0.19-0.25 | 0.74-1.21 | 0.20-0.27 | This study |
| | pathway | | Heterologous expression of xylD from | | | | |
| | | | Caulobacter crescentus | | | | |
| | | | Adaptive laboratory evaluation in | | | | |
| | | | a xylose medium | | | | |
| Xylose | [symbol] | Plasmid | Host: P. putida KT2440 | 0.21 | 0.22-0.46 | — | (Bator et al., |
| | pathway | | Heterologous expression of PVLB18555 | | | | 2019) |
| | | | and PVLB18565 from P. [symbol] VLB120 | | | | |
| Galactose | De Ley- | Chromosome | Host: P. putida KT2440 | 0.37 | — | — | (Peabody et al., |
| | Doudoroff | | Heterologous expression of [symbol] AB | | | | 2019) |
| | pathway | | from [symbol] [symbol] , [symbol] KAD | | | | |
| | | | from P. fluorescens SBW25, [symbol] AB and | | | | |
| | | | galP from E. coli | | | | |
| Galactose | Leloir | Chromosome | Host: P. putida KT2440 | <0.1 | — | — | (Banerjee et al., |
| | pathway | | Heterologous expression of galETKM | | | | 2020) |
| | | | from E. coli | | | | |
| Galactose | Leloir | Chromosome | Host: P. putida KT2440 | 0.37-0.52 | 1.30-2.55 | 0.15-0.32 | This study |
| | pathway | | Heterologous expression on galETKM | | | | |
| | | | from E. coli | | | | |
| | | | Adaptive laboratory evolution in a | | | | |
| | | | galactose medium | | | | |

[symbol]

indicates data missing or illegible when filed

While it was shown that KT2440 can be engineered to utilize xylose and galactose, several limitations remain. We currently have insufficient understanding of which endogenous genes are involved in the sugar catabolism. Furthermore, initial studies have expressed heterologous genes using plasmids, which is not ideal and preferable for industry-scale cultivations, as plasmid-based expression increases genotypic or phenotypic instability due to its heterogeneous nature (Elmore et al., 2020; Kang et al., 2018). However, genome-based engineering often resulted in unsatisfactory cell growth (i.e., slow growth rate or long lag phase) which makes their practical deployments challenging. The successful activation of non-native sugar utilization pathways with chromosomal expression has been demonstrated for only the xylose isomerase pathway (Elmore et al., 2020) and De Ley-Doudoroff pathway (Peabody et al., 2019) (Table 2). Given each utilization pathway generates different intermediates and biochemical yields (Bator et al., 2019), further studies to construct less-explored pathways in KT2440 are warranted.

In recent decades, the approach of adaptive laboratory evolution (ALE) has shown significant potential to generate useful strains for industry-relevant purposes (Sandberg et al., 2019). The continuous cell culture and growth-based selection allow for the accumulation of beneficial mutations for improved fitness under a given condition. The recent development of automated ALE platforms (LaCroix et al., 2017; Wong et al., 2018) enabled multiplexity and controllability to change environments dynamically (e.g., substrate feeding). In addition to the strain generation, the increased accessibility to the next-generation sequencing allows for the rapid identification of genomic and transcriptomic variations in evolved cells, providing hints to understand mutational mechanisms. Indeed, many strains with industry-relevant phenotypes (e.g., higher tolerance, substrate utilization) (Guzmán et al., 2019; Lim et al., 2020; Mohamed et al., 2020, 2019, 2017; Nguyen-Vo et al., 2019; Reider Apel et al., 2016) have been generated and their mutational mechanisms were also suggested or validated through reverse engineering. In this regard, it was expected that the ALE approach and related analysis has the potential to generate strains with improved sugar utilization and that the resulting mutational mechanisms could lead to a deeper understanding of how strains can optimize novel phenotypes related to the introduction of heterologous pathways.

### SUMMARY OF THE INVENTION

The present invention provides for a Pseudomonas cell is able to grow in a medium with xylose or galactose as a sole carbon source with a growth rate of equal to or higher than 0.10 h⁻¹.

In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P, oryzihabitans, P. parafluva, or P. plecoglossicida. In some embodiments, the P. putida is strain KT2440.

In the embodiments of the Pseudomonas cell capable of utilizing xylose and/or galactose as a sole carbon source, the Pseudomonas cell comprises the following genes: (A) a gene encoding a heterologous xylonate dehydratase (such as the xylD gene, such the Caulobacter crescentus xylD gene), and mutations in native ptxS and/or kguT genes encoding a 2-ketogluconate operon repressor and 2-ketogluconate transporter, respectively; and/or, (B) genes encoding heterologous UDP-glucose 4-epimerase, galactose-1-phosphate uridylyltransferase, galactokinase, and galactose-1-epimerase (such as the galETKM genes, such the Escherichia coli galETKM genes), and mutation(s) in a native gtsABCD gene encoding an ATP-binding cassette (ABC) sugar transporting system.

In the embodiments of the Pseudomonas cell comprises one or more of the following mutations in the chromosome: ptxS, kguT, gacS, ftsH, PP_4173, galP-I/PP_1174, gtsABCD, oprB-1I/yeaD, and oprB-II as described herein.

In the embodiments of the Pseudomonas cell capable of utilizing xylose as a sole carbon source, the Pseudomonas cell comprises the following genes: a gene encoding xylonate dehydratase (such as the xylD gene, such the Caulobacter crescentus xylD gene), and mutations in ptxS and/or kguT genes encoding a 2-ketogluconate operon repressor and 2-ketogluconate transporter, respectively. In some embodiments, the Pseudomonas cell growing in a medium with xylose as a sole carbon source has a growth rate of equal to or higher than 0.10 h⁻¹, 0.15 h⁻¹, 0.20 h⁻¹, or 0.25 h⁻¹.

In the embodiments of the Pseudomonas cell capable of utilizing galactose as a sole carbon source, the Pseudomonas cell comprises the following genes: genes encoding UDP-glucose 4-epimerase, galactose-1-phosphate uridylyltransferase, galactokinase, and galactose-1-epimerase (such as the galETKM genes, such the Escherichia coli galETKM genes), and mutation(s) in gtsABCD genes encoding an ATP-binding cassette (ABC) sugar transporting system. In some embodiments, the Pseudomonas cell growing in a medium with galactose as a sole carbon source has a growth rate of equal to or higher than 0.10 h⁻¹, 0.15 h⁻¹, 0.20 h⁻¹, 0.25 h⁻¹, 0.30 h⁻¹, 0.35 h⁻¹, 0.40 h⁻¹, 0.45 h⁻¹, 0.50 h⁻¹, or 0.52 h⁻¹.

In some embodiments, the native ptxS, kguT, and/or gtsABCD genes are each independently knocked out or deleted from the chromosome.

In some embodiments, the xylD gene and/or the galETKM genes are each independently capable of expression from the chromosome under a constitutive promoter and 5′-untranslated region.

In some embodiments, the mutations in ptxS and/or kguT genes encoding a 2-ketogluconate operon repressor and 2-ketogluconate transporter, respectively, and/or the mutation(s) in gtsABCD genes encoding an ATP-binding cassette (ABC) sugar transporting system are described herein and shown in FIG. 3A to FIG. 3D. In some embodiments, the Pseudomonas cell comprises one or more of the mutations described herein and shown in FIG. 3A to FIG. 3D. When the mutation shown in FIG. 3A to FIG. 3D as an amino acid substitution, the actual mutation is one or more substitutions of the corresponding nucleotides in the corresponding gene that results in the amino acid(s) substituted.

The present invention provides for an engineered Pseudomonas bacterium comprising at least one heterologous protein(s) of the Weimberg pathway, and at least one mutation(s) in a chromosome of the bacterium.

In some embodiments, the at least one heterologous protein(s) comprises xylonate dehydratase. In some embodiments, the at least one heterologous protein(s) comprises xylonate dehydratase of a species selected from Caulobacter crescentus, Caulobacter vibrioides, or Halomonas elongate. In some embodiments, the xylonate dehydratase is encoded by a heterologous xylD gene. In some embodiments, the bacterium is Pseudomonas putida KT2440. In some embodiments, the bacterium catabolizes xylose. In some embodiments, the at least one mutation(s) comprises a mutation in a gene(s) or intergenic region(s) selected from the group of: betT-II; cysQ; mmsA-I, PP_16SD; ettA; phnC; PP_1028; xcpUlxcpV; PP_1104; galP-I, PP_1174; yhdX; yhdY; PP_1475; mutS; gacS; apeB; PP_1886; PP_1980, dusC; PP_2222; PP_2277; PP_2287; PP_2411; sad-I, PP_2489; PP_2628; PP_2851; PP_2855, PP_2856; PP_2962, PP_2964; dnaEB; kguT; ptxS; PP_3475; PP_3573; PP_3645; creA, katG; ribAB-II; PP_4085; uvrY; PP_4173; fliI; ooxB; ftsH; PP_4824; parC; PP_4926; PP_4950; PP_4983; PP_5043; betT-I; PP_5167, cysA; PP_5729, rpmG; the intergenic region between galP-1 and PP_1174 (noted as galP-I/PP_1174); or any other gene or intergenic region as disclosed herein.

In some embodiments, the at least one mutation(s) comprises a mutation in one or more of gene(s) or intergenic region(s) selected from the group of: galP-I, PP_1174; gacS; kguT; ptxS; PP_4173; or ftsH. In some embodiments, the at least one mutation(s) comprises a mutation(s) disclosed in Sheet 3 of Supplementary Data 3 (filed in U.S. Provisional Patent Application Ser. Nos. 63/168,687, filed on Mar. 31, 2021). In some embodiments, the at least one mutation(s) comprises one or more of gain-of-function mutation(s) in genes selected from the group of: kguE, kguK, kguT, ptxD, gcd or another gene a gene product of which transports xylonate. In some embodiments, the at least one mutation(s) comprises one or more of loss-of-function mutation(s) in ptzS. In some embodiments, the engineered bacterium comprises an altered expression level of one or more of the genes disclosed in Sheet 6 of Supplementary Data 3 (filed in U.S. Provisional Patent Application Ser. Nos. 63/168,687, filed on Mar. 31, 2021) compared to the wildtype grown on glucose or the bacterium without the at least one mutation(s) grown on glucose. In some embodiments, the engineered bacterium comprises an altered expression level of one or more of the genes disclosed in Sheet 7 of Supplementary Data 3 (filed in U.S. Provisional Patent Application Ser. Nos. 63/168,687, filed on Mar. 31, 2021) compared to the wildtype grown on glucose or the bacterium without the at least one mutation(s) grown on glucose. In some embodiments, the engineered bacterium comprises an altered expression level of one or more of the genes disclosed in Sheet 8 of Supplementary Data 3 (filed in U.S. Provisional Patent Application Ser. Nos. 63/168,687, filed on Mar. 31, 2021) compared to the wildtype grown on glucose or the bacterium without the at least one mutation(s) grown on glucose. In some embodiments, the engineered bacterium comprises an increased expression level of one or more of genes selected from the group of: gnl (PP_1170), galP-I (PP_1173), PP_2585, fucD (PP_2831), PP_2834, PP_2835. PP_2836, PP_2837, ptxD (PP_3376), kguT (PP_3377), kguK (PP_3378), kguE (PP_3379), ptxS (PP_3380), or pyk (PP_4301), compared to the wildtype grown on glucose or the bacterium without the at least one mutation(s) grown on glucose. In some embodiments, the engineered bacterium comprises a decreased expression level of one or more of genes selected from the group of: gapA (PP_1009), edd (PP_1010), glk (PP_1011), gtsA (PP_1015), gtsB (PP_1016), gtsC (PP_1017), gtsD (PP_1018), yeaD (PP_1020), zwfA (PP_1022), eda (PP_1024), pykA (PP_1362), gcd (PP_1444), oprB-II (PP_1445), eno (PP_1612), PP_3382, PP_3383, PP_3384, gnuK (PP_3416), gntT (PP_3417), oprB-III (PP_3570), or zwfB (PP_4042), compared to the wildtype grown on glucose or the bacterium without the at least one mutation(s) grown on glucose. In some embodiments, the engineered bacterium has a growth rate in a range from 0.20 h⁻¹ to 0.26 h⁻¹, or 0.22 h⁻¹ to 0.25 h⁻¹, or any range or rate of each therebetween. In some embodiments, the engineered bacterium has a xylose uptake catalytic rate up to 1.2 g per g of dry cell weight (DCW) per hour. In some embodiments, the engineered bacterium has a biomass yield equal to or more than 0.20 g xylose per g of DCW. In some embodiments, the engineered bacterium further comprises one or both of the following in a chromosome of the bacterium: a nucleic acid sequence encoding the heterologous protein(s), or a regulatory sequence directing expression of the heterologous protein in the bacterium optionally a constitutive promoter and 5′-untranslated region. In some embodiments, the gene mutation(s) is engineered into the bacterium by a plasmid or by culturing a bacterium comprising the at least one heterologous protein(s) of the Weimberg pathway in a culture medium comprising xylose as the sole carbon source or one of the carbon sources.

The present invention provides for an engineered Pseudomonas bacterium, comprising at least one heterologous protein(s) of the Leloir pathway and at least one mutation(s) in a chromosome of the bacterium.

In some embodiments, the at least one heterologous protein(s) comprises one or more of proteins selected from the group of: UDP-glucose 4-epimerase, galactose-1-phosphate uridylyltransferase, galactokinase, or galactose-1-epimerase. In some embodiments, the at least one heterologous protein(s) comprises one or more of Escherichia coli K-12 MG1655 proteins selected from the group of: UDP-glucose 4-epimerase, galactose-1-phosphate uridylyltransferase, galactokinase, or galactose-l-epimerase. In some embodiments, the one or more of proteins is encoded by a heterologous galETKM operon. In some embodiments, the engineered bacterium is Pseudomonas putida KT2440. In some embodiments, the engineered bacterium catabolizes galactose. In some embodiments, the at least one mutation(s) comprises a mutation in a gene(s) or intergenic region(s) selected from the group of: PP_0501; Ecoli_galK; Ecoli_galE, prfC; colS; PP_0949; rpoN; PP_1013; gtsA; gtsC; gtsB; gtsD; gtsABCD; galETKM; oprB-I; oprB-1, yeaD (oprB-I/yeaD); PP_1033, cumA; zapE; PP_1366; oprB-II; mutS; PP_1770; rffE; PP_1948; pepN; cmpX; folD-II; clpP; PP_2750; PP_2907; tnpT-I; gpD; PP_3935, PP_3938; uvrY; PP_4171; pvdL; fliO; flgH; PP_4592; PP_4684; dnaJ; or any other gene or intergenic region as disclosed herein. In some embodiments, the at least one mutation(s) comprises a mutation in a gene(s) or an intergenic region(s) selected from the group of: gtsA; gtsC; oprB-I; oprB-I, yeaD; or oprB-II. In some embodiments, the at least one mutation(s) comprises a mutation(s) disclosed in Sheet 4 of Supplementary Data 3 (filed in U.S. Provisional Patent Application Ser. Nos. 63/168,687, filed on Mar. 31, 2021). In some embodiments, the at least one mutation(s) comprises one or more of gain-of-function mutation(s) in genes selected from the group of: gtsA; gtsC; gtsB; gtsD; gtsABCD; or another gene, a gene product of which transports galactose into the cytosol. In some embodiments, the at least one mutation(s) comprises one or more of loss-of-function mutation(s) in oprB-I/yeaD or oprB-II or both. In some embodiments, the engineered bacterium comprises an altered expression level of one or more of the genes disclosed in one or more of Sheet 6 of Supplementary Data 3 (filed in U.S. Provisional Patent Application Ser. Nos. 63/168,687, filed on Mar. 31, 2021), compared to the wildtype grown on glucose or the bacterium without the at least one mutation(s) grown on glucose. In some embodiments, the engineered bacterium comprises an altered expression level of one or more of the genes disclosed in Sheet 7 of Supplementary Data 3 (filed in U.S. Provisional Patent Application Ser. Nos. 63/168,687, filed on Mar. 31, 2021) compared to the wildtype grown on glucose or the bacterium without the at least one mutation(s) grown on glucose. In some embodiments, the engineered bacterium comprises an altered expression level of one or more of the genes disclosed in Sheet 8 of Supplementary Data 3 (filed in U.S. Provisional Patent Application Ser. Nos. 63/168,687, filed on Mar. 31, 2021) compared to the wildtype grown on glucose or the bacterium without the at least one mutation(s) grown on glucose. In some embodiments, the engineered bacterium comprises an increased expression level of one or more of genes selected from the group of: gtsABCD; gapA (PP_1009); edd (PP_1010); glk (PP_1011); gtsA (PP_1015); gtsB (PP_1016): gtsC (PP_1017); gtsD (PP_1018); oprB-I (PP_1019); gnl (PP_1170); galP-I (PP_1173); pykA (PP_1362); PP_2585; fucD (PP_2831); PP_2834; PP_2835; PP_2836; PP_2837; or pyk (PP_4301), compared to the wildtype grown on glucose or the bacterium without the at least one mutation(s) grown on glucose. In some embodiments, the engineered bacterium comprises a decreased or increased expression level of oprB-II or gdc or both compared to the wildtype grown on glucose or the bacterium without the at least one mutation(s) grown on glucose. In some embodiments, the engineered bacterium comprises a decreased expression level of one or more of the following genes compared to the wildtype grown on glucose or the bacterium without the at least one mutation(s) grown on glucose: ptxD (PP_3376); kguT (PP_3377); kguK (PP_3378); kguE (PP_3379); ptxS (PP_3380); PP_3382; PP_3383; PP_3384; gnuK (PP_3416); gntT (PP_3417); oprB-III (PP_3570); or zwfB (PP_4042). In some embodiments, the engineered bacterium has a growth rate in a range from 0.33 h⁻¹ and 0.52 h⁻¹, or any range or rate of each therebetween. In some embodiments, the engineered bacterium further comprises one or both of the following in a chromosome of the bacterium: a nucleic acid sequence encoding the heterologous protein(s), or a regulatory sequence directing expression of the heterologous protein in the bacterium optionally a constitutive promoter and 5′-untranslated region. In some embodiments, the gene mutation(s) is engineered into the bacterium by a plasmid or by culturing a bacterium comprising the at least one heterologous protein(s) of the Leloir pathway in a culture medium comprising galactose as the sole carbon source or one of the carbon sources. In some embodiments, the engineered bacterium further comprises a heterologous gene expressing a gene product. In some embodiments, the gene product is one or more enzymes producing indigoiodine.

The present invention provides for a plurality of the engineered bacterium of the present invention. In some embodiments, the bacterium in the plurality are the same or different from each other.

The present invention provides for a composition comprising the plurality of bacterium of is the present invention, and a carrier.

The present invention provides for a method for producing a metabolite comprising culturing the engineered bacterium of the present invention in a culture medium.

In some embodiments, the method further comprises isolating the metabolite from the culture medium or the bacterium.

The present invention provides for a method for producing a metabolite comprising culturing a bacterium of the present invention in a culture medium, wherein the heterologous gene expressing an enzyme catalyzing and producing the metabolite.

In some embodiments, the method further comprises isolating the metabolite from the culture medium or the bacterium.

The present invention provides for a method for generating an engineered bacterium, comprising (a) culturing a bacterium that comprises at least one heterologous protein(s) catabolizing a non-native carbon source in a culture medium, wherein the culture medium comprises the non-native carbon source as the sole carbon source; (b) monitoring growth rate of the cultured bacterium; and (c) isolating a progeny of the cultured bacterium when or prior to the growth rate reaching a Plateau.

The present invention provides for method for generating an engineered bacterium, comprising (a) culturing a bacterium that comprises at least one heterologous protein(s) catabolizing a non-native carbon source in a series of culture medium, wherein the series of culture medium comprises a gradually increased ratio of the non-native carbon source over a native carbon source; (b) monitoring growth rate of the cultured bacterium; and (c) isolating a progeny of the cultured bacterium when or prior to the growth rate reaching a Plateau.

The present invention provides for an engineered bacterium generated by a method of the present invention.

The present invention provides for an engineered bacterium as disclosed herein.

The present invention provides for an engineered bacterium for use in biomass processing processes.

The present invention provides for a kit for use in a method of the present invention comprising an engineered bacterium of the present invention or a plurality thereof, and instructions.

The present invention provides for a kit for use in a method of the present invention comprising the bacterium that comprises at least one heterologous protein(s), the non-native carbon, and instructions.

The present invention provides for an engineered or isolated polynucleotide comprising a mutated gene as disclosed in Supplementary Data 3 (filed in U.S. Provisional Patent Application Ser. Nos. 63/168,687, filed on Mar. 31, 2021).

The present invention provides for an engineered or isolated polypeptide comprising a mutated protein as disclosed in Supplementary Data 3 (filed in U.S. Provisional Patent Application Ser. Nos. 63/168,687, filed on Mar. 31, 2021).

In one aspect, provided herein is an engineered Pseudomonas bacterium catabolizing xylose (i.e., utilizing xylose as a carbon source), comprising at least one heterologous protein(s) of the Weimberg pathway and at least one mutation(s) in a gene of the Weimberg pathway. Also provided herein is an engineered Pseudomonas bacterium catabolizing galactose (i.e., utilizing galactose as a carbon source), comprising at least one heterologous protein(s) of the Leloir pathway and at least one mutation(s) in a gene of the Leloir pathway. Further, provided herein is an engineered Pseudomonas bacterium catabolizing xylose or galactose or both (i.e., utilizing xylose or galactose or both as a carbon source), comprising at least one heterologous protein(s) of the Weimberg pathway or the Leloir pathway or both and at least one mutation(s) in a gene of the Weimberg pathway or the Leloir pathway or both.

Additionally provided are aspects or embodiments of the engineered bacterium as disclosed herein, methods of using the engineered bacterium, methods of producing the engineered bacterium, compositions made by the bacterium and related compositions or kits containing these.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. Adaptive Laboratory Evolution (ALE) strategies for improving the xylose and galactose utilization. The ALE strategies to evolve the P. putida xylD and P. putida galETKM strains.

FIG. 1B. Adaptive Laboratory Evolution (ALE) strategies for improving the xylose and galactose utilization. The ALE strategies to evolve the P. putida xylD and P. putida galETKM strains.

FIG. 1C. Growth trajectories of ALE1-4 with the xylD and galETKM strains, respectively. The x-axis and y-axis indicate Cumulative Cell Divisions (CCD) (Lee et al., 2011) and the maximum specific growth rate (h⁻¹). CCD for the galactose ALE experiments were calculated from the first flask which displayed an observable growth rate solely on galactose minimal media.

FIG. 1D. Growth trajectories of ALE5, ALE6, and ALE8 with the xylD and galETKM strains, respectively. The x-axis and y-axis indicate Cumulative Cell Divisions (CCD) (Lee et al., 2011) and the maximum specific growth rate (h⁻¹). CCD for the galactose ALE experiments were calculated from the first flask which displayed an observable growth rate solely on galactose minimal media.

FIG. 1 Comparisons of the maximum specific growth rates (h⁻¹) of isolated clones from different evolutionary timepoints of ALE1-4. Cell cultures were conducted in biological duplicates and error bars indicate the minimum and maximum values.

FIG. 1E. Comparisons of the maximum specific growth rates (h⁻¹) of isolated clones from different evolutionary timepoints of ALE5, ALE6, and ALE8. Cell cultures were conducted in biological duplicates and error bars indicate the minimum and maximum values.

FIG. 2A. Growth profiles of the wildtype KT2440 and evolved isolates. Growth and biomass yields of the P. putida xylD strain and evolved isolates in the xylose minimal medium during a 24 h cultivation. x-axis indicates time (h) and y-axis indicates OD₆₀₀. The cultures were conducted with three biological replicates (n=3) and error bars indicate the standard deviations.

FIG. 2B. Growth profiles of the wildtype KT2440 and evolved isolates. Xylose consumption and biomass yields of the P. putida xylD strain and evolved isolates in the xylose minimal medium during a 24 h cultivation. x-axis indicates time (h) and y-axis indicates sugar concentration (g/L). The cultures were conducted with three biological replicates (n=3) and error bars indicate the standard deviations.

FIG. 2C. Growth profiles of the wildtype KT2440 and evolved isolates. Xylose uptake rates and biomass yields of the P. putida xylD strain and evolved isolates in the xylose minimal medium during a 24 h cultivation. Left and right y-axis indicates sugar consumption rate during the exponential growth phase and biomass yield, respectively. The cultures were conducted with three biological replicates (n=3) and error bars indicate the standard deviations.

FIG. 2D. Growth profiles of the wildtype KT2440 and evolved isolates. Growth and biomass yields of the P. putida galETKM strain and evolved isolates on galactose in the galactose minimal medium during a 24 h cultivation. x-axis indicates time (h) and left y-axis indicates OD₆₀₀. The cultures were conducted with three biological replicates (n=3) and error bars indicate the standard deviations.

FIG. 2E. Growth profiles of the wildtype KT2440 and evolved isolates. Galactose consumption and biomass yields of the P. putida galETKM strain and evolved isolates on galactose in the galactose minimal medium during a 24 h cultivation. x-axis indicates time (h) and left y-axis indicates sugar concentration (g/L). The cultures were conducted with three biological replicates (n=3) and error bars indicate the standard deviations.

FIG. 2F. Growth profiles of the wildtype KT2440 and evolved isolates. Galactose uptake rates and biomass yields of the P. putida galETKM strain and evolved isolates on galactose in the galactose minimal medium during a 24 h cultivation. Left and right y-axis indicates sugar consumption rate during the exponential growth phase and biomass yield, respectively. The cultures were conducted with three biological replicates (n=3) and error bars indicate the standard deviations.

FIG. 3A. Genomic and transcriptomic analysis of evolved isolates. Identified mutations in the ptxS-kguEKT-ptxD region of evolved P. putida xylD clones. Uppercase and lowercase letters indicate amino acids and nucleobases, respectively. * indicates the early termination mutation. Arrow sizes do not represent gene lengths. Colors: blue, amino acid deletions, frame shift mutations, early termination mutations; green, synonymous mutations; purple, single nucleotide mutations; orange, single amino acid changes.

FIG. 3B. Genomic and transcriptomic analysis of evolved isolates. Identified mutations in the gtsA-gtsBCD-oprB-I-yeaD and oprB-II-gcd regions of evolved P. putida galETKM clones. Uppercase and lowercase letters indicate amino acids and nucleobases, respectively. * indicates the early termination mutation. Arrow sizes do not represent gene lengths. Colors: blue, amino acid deletions, frame shift mutations, early termination mutations; green, synonymous mutations; purple, single nucleotide mutations; orange, single amino acid changes.

FIG. 3C. Central carbon metabolism of KT2440 with the heterologous xylose and galactose utilization genes. Red and blue arrows indicate the Weimberg and Leloir pathways, respectively. Heterologous genes were colored in red or blue. Abbreviations: 2-KG, 2-ketogluconate; G6P, glucose-6-phosphate (P); 6PG, 6-phosphogluconate; 2KG6P, 2-ketogluconate-6-P; Gal1P, galactose-1-P; UDP-Glc, uridine diphosphate-glucose; UDP-Gal, uridine diphosphate galactose; G1P, glucose-1-P; KDPG, 2-dehydro-3-deoxy-phosphogluconate; PYR, pyruvate; G3P, glyceraldehyde-3-P; 3PG, glycerate-3-P; 2PG, 2-phosphoglycerate; PEP, phosphoenolpyruvate; acetyl-CoA, acetyl coenzyme A (CoA), CIT, citrate; ICT, isocitrate; aKG, α-ketoglutarate; SUC-CoA, succinyl-CoA; SUC, succinate; FUM, fumarate; MAL, malate; OAA, oxaloacetate; GLY, glyoxylate.

FIG. 3D. Log₂ Transcripts Per Million (TPM) fold changes of genes related to sugar catabolism. Actual values were provided in Supplementary Data 3 (filed in U.S. Provisional Patent Application Ser. Nos. 63/168,687, filed on Mar. 31, 2021).

FIG. 4A. Validation of xylose and galactose metabolism in evolved strains. Growth profiles of the A1_F11_I1 (black circle), A1_F11_I1_ΔxylD (red square), and A1_F11_I1_ΔkguT (green down triangle) strains in the xylose minimal medium. These strains were cultivated using a microtiter plate reader. x-axis indicates time (h). y-axis indicates OD₆₀₀. The cultures were conducted with three biological replicates (n=3) and error bars indicate the standard deviations.

FIG. 4B. Validation of xylose and galactose metabolism in evolved strains. Growth profiles of the A6_F90_I1 (black circle), A6_F90_I1_ΔgalETKM (red square), and A6_F90_I1_ΔgtsABCD (green down triangle) strains in the galactose minimal medium. These strains were cultivated using a microtiter plate reader. x-axis indicates time (h). y-axis indicates OD₆₀₀. The cultures were conducted with three biological replicates (n=3) and error bars indicate the standard deviations.

FIG. 4C. Validation of xylose and galactose metabolism in evolved strains. Growth of A5_F85_I1 (black open circle), A5_F85_I1_Δgcd (black closed circle), A8_F92_I1 (red open square), A8_F92_I1_Δgcd (red closed square) strains. x-axis indicates time (h). y-axis indicates OD₆₀₀. The cultures were conducted with three biological replicates (n=3) and error bars indicate the standard deviations.

FIG. 4D. Validation of xylose and galactose metabolism in evolved strains. Sugar consumption profiles of A5_F85_I1 (black open circle), A5_F85_I1_Δgcd (black closed circle), A8_F92_I1 (red open square), A8_F92_I1_Δgcd (red closed square) strains. x-axis indicates time (h). y-axis indicates galactose concentration (g/L). The cultures were conducted with three biological replicates (n=3) and error bars indicate the standard deviations.

FIG. 5A. Indigoidine production from xylose and galactose by using evolved strains as hosts. The indigoidine production pathway engineered into P. putida strains. Indigoidine can be produced by heterologous expression of bpsA from Streptomyces lavendulae and sfp from Bacillus subtilis for conversion of glutamine. These genes were expressed under the arabinose inducible promoter (Pₐᵣₐ).

FIG. 5B. Indigoidine production from xylose and galactose by using evolved strains as hosts. Comparison of the indigoidine titers (g/L) of the initial engineered strains (not detected, n.d.), evolved isolates, and two evolved isolates with a gcd deletion after 24 h (light blue) and 48 h (dark blue) cultivation. The bpsA and sgfp expression cassette was integrated into the chromosome of each host strain. Four biological replicates (n=4) were performed, and error bars indicate the standard deviations.

FIG. 6. Pathways for glucose, xylose, and galactose utilization. Glucose, xylose, and galactose utilization pathways found in microorganisms. Glucose is metabolized by Embden-Meyerhof-Parnas pathway (EMP pathway) or Entner-Doudorof pathway (ED pathway), or their combination. For xylose utilization, there are 4 known pathways: isomerase pathway, oxidoreductase pathway, Dahms pathway, and Weimberg pathway. For galactose utilization, Leloir pathway and De Ley-Doudoroff pathway have been reported. Dashed lines indicate missing enzymatic steps in Pseudomonas putida KT2440.

FIG. 7A. Growth profiles of the wildtype KT2440 and engineered strains on xylose and galactose. Growth profiles of the wildtype and P. putida xylD strain on xylose. The cultures were conducted in 200 μL of the minimal media supplemented with 4 g/L of either xylose or galactose as a sole carbon source. x-axis and y-axis indicate time (h) and OD₆₀₀, respectively. The cultures were conducted with three biological replicates (n=3) and error bars indicate the standard deviations.

FIG. 7B. Growth profiles of the wildtype KT2440 and engineered strains on xylose and galactose. Growth profiles of the wildtype and P. putida galETKM strain on galactose. The cultures were conducted in 200 μL of the minimal media supplemented with 4 g/L of either xylose or galactose as a sole carbon source. x-axis and y-axis indicate time (h) and OD₆₀₀, respectively. The cultures were conducted with three biological replicates (n=3) and error bars indicate the standard deviations.

FIG. 8. Growth rate comparison of the wildtype KT2440 and evolved strains on glucose.

FIG. 9A. I-TASSER predicted structures of KguT, GtsA, and GtsC and their mutations. Predicted structures of KguT GtsC of P. putida KT2440 using I-TASSER (Yang and Zhang, 2015). Blue colored residues are predicted substrate binding residues by the software. Mutated residues are colored in red. Mutated residues which were predicted to be substrate binding residues, were colored in purple.

FIG. 9B. I-TASSER predicted structures of KguT, GtsA, and GtsC and their mutations. Predicted structures of GtsA of P. putida KT2440 using I-TASSER (Yang and Zhang, 2015). Blue colored residues are predicted substrate binding residues by the software. Mutated residues are colored in red. Mutated residues which were predicted to be substrate binding residues, were colored in purple. Glucose was additionally shown.

FIG. 9C. I-TASSER predicted structures of KguT, GtsA, and GtsC and their mutations. Predicted structures of GtsC of P. putida KT2440 using I-TASSER (Yang and Zhang, 2015). Blue colored residues are predicted substrate binding residues by the software. Mutated residues are colored in red. Mutated residues which were predicted to be substrate binding residues, were colored in purple.

FIG. 10A. Cluster of orthologous groups (COG) analysis of commonly differentially expressed genes. Cluster of orthologous groups (COG) analysis of commonly differentially expressed genes in a xylose medium. A, RNA processing and modification; B, chromatin structure and dynamics; C, energy production and conversion; D, cell cycle control and mitosis; E, amino acid metabolism and transport; F, nucleotide metabolism and transport; G, carbohydrate metabolism and transport; H, coenzyme metabolism; I, lipid metabolism; J, translation; K, transcription; L, replication and repair; M, cell wall/membrane/envelop biogenesis; N, cell motility; O, post-translational modification; P, inorganic ion transport and metabolism; Q, secondary structure; R, general functional prediction only; S, function unknown; T, signal transduction; U, intracellular trafficking; V, defense mechanism; W, extracellular structure; Y, nuclear structure; Z, cytoskeleton. Uncharacterized 346 (xylose) and 543 (galactose) genes were not displayed.

FIG. 10B. Cluster of orthologous groups (COG) analysis of commonly differentially expressed genes. Cluster of orthologous groups (COG) analysis of commonly differentially expressed genes in a galactose medium. A, RNA processing and modification; B, chromatin structure and dynamics; C, energy production and conversion; D, cell cycle control and mitosis; E, amino acid metabolism and transport; F, nucleotide metabolism and transport; G, carbohydrate metabolism and transport; H, coenzyme metabolism; I, lipid metabolism; J, translation; K, transcription; L, replication and repair; M, cell wall/membrane/envelop biogenesis; N, cell motility; O, post-translational modification; P, inorganic ion transport and metabolism; Q, secondary structure; R, general functional prediction only; S, function unknown; T, signal transduction; U, intracellular trafficking; V, defense mechanism; W, extracellular structure; Y, nuclear structure; Z, cytoskeleton. Uncharacterized 346 (xylose) and 543 (galactose) genes were not displayed.

FIG. 11. Transcripts per million of the galETKM genes and their neighboring genes. Transcripts per million of pfrC, galE, gaT, galK, galM, PP_0871 genes.

FIG. 12. Standard curve for indigoidine quantification. Indigoidine was dissolved at various concentrations in the minimal medium and the absorbance at 612 nm was measured. Error bars indicate standard deviations of three technical replicates.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

The term “about” refers to a value including 1%, 5%, or 10% more than the stated value and 10% less than the stated value.

The term “substantially” or “essentially” means nearly totally or completely, for instance, 95% or greater of some given quantity. In some embodiments. “substantially” or “essentially” means 95%, 96%, 97%, 98%, 99%, 99.5%, or 99.9% or greater of some given quantity.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “host cell” is used herein to refer to a living biological cell that can be transformed via insertion of an expression vector.

The term “heterologous” as used herein refers to a material, or nucleotide or amino acid sequence, that is found in or is linked to another material, or nucleotide or amino acid sequence, wherein the materials, or nucleotide or amino acid sequences, are foreign to each other (i.e., not found or linked together in nature).

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

A polynucleotide is “heterologous” to a host cell or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “native” refers to two structures that are found together in nature, such as within the same naturally-occurring organism or naturally-occurring structure.

The term “operatively linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

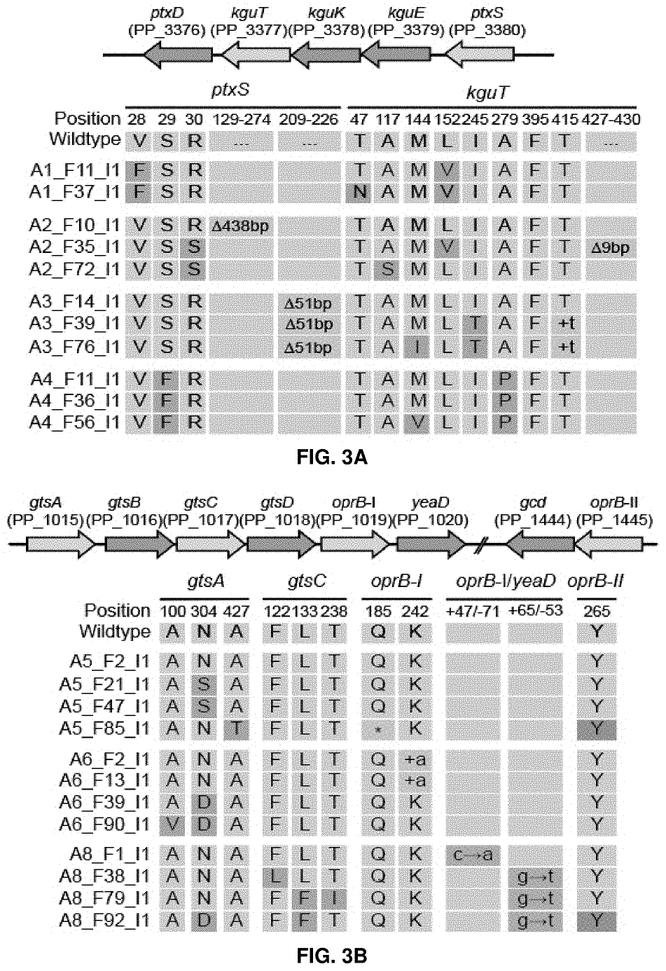

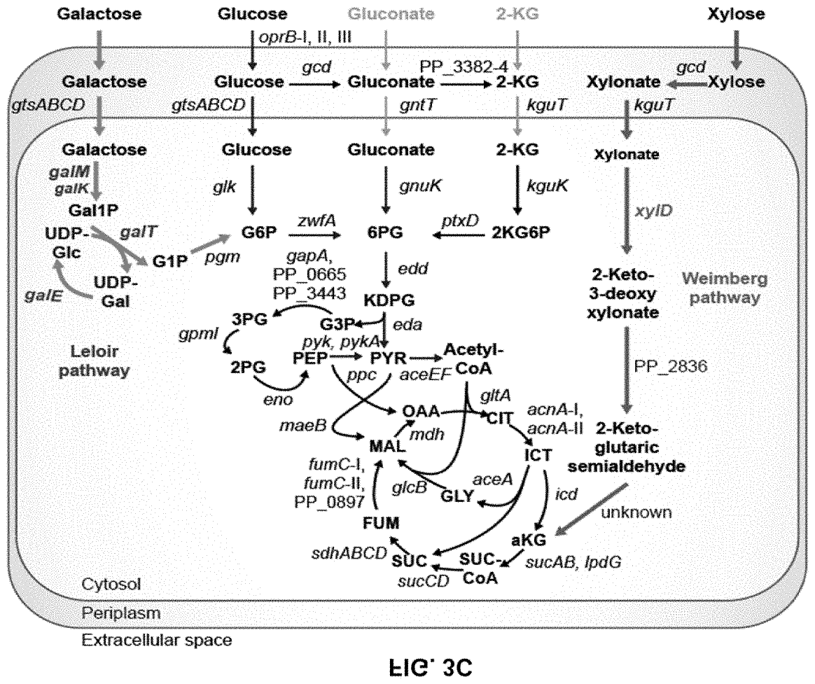

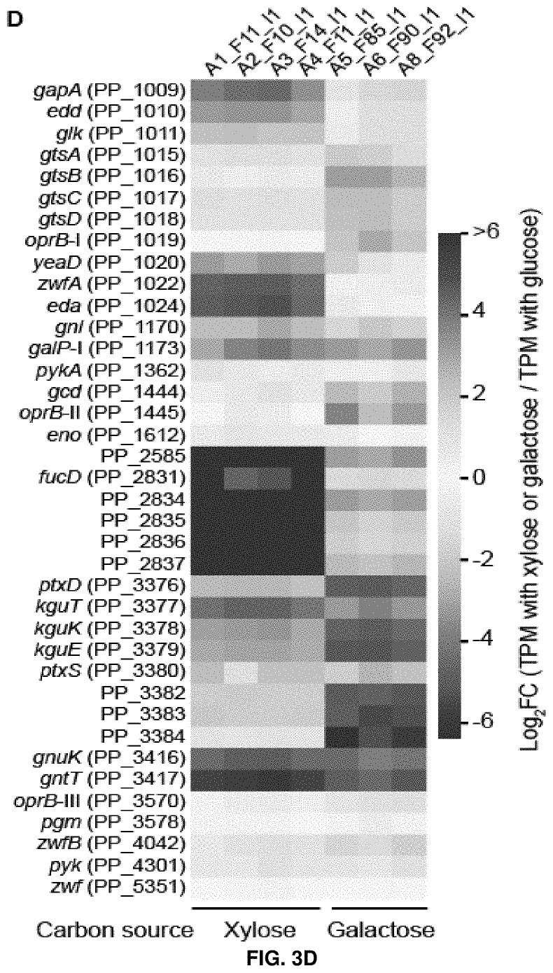

The Weimberg pathway is an oxidative pathway where a D-xylose is oxidized to D-xylono-lactone by a D-xylose dehydrogenase followed by a lactonase to hydrolyze the lactone to D-xylonic acid. A xylonate dehydratase is splitting off a water molecule resulting in 2-keto 3-deoxy-xylonate. A second dehydratase forms the 2-keto glutarate semialdehyde which is subsequently oxidized to 2-ketoglutarate. An illustration of the Weimberg pathway is provided in FIGS. 3 and S1 in the Appendices (filed in U.S. Provisional Patent Application Ser. Nos. 63/168,687, filed on Mar. 31, 2021). In some embodiments, the Weimberg pathway comprises, or consists essentially of, or yet further consists of one or more of the following steps: Xylose is transported or diffused from the extracellular space into the periplasm, converted to Xylonate (e.g., by gcd), transported to cytosol (e.g., by kguT), converted to 2-keto-3-deoxy-xylonate (e.g., by xylD), further converted to 2-Ketoglutaric semialdehyde (e.g., by PP_2836) and then to a-ketoglutarate (i.e., aKG or aKG).

The Leloir pathway is a metabolic pathway for the catabolism of D-galactose. In some embodiments: the first step in the Leloir pathway comprises, or consists essentially of, or yet further consists of galactose mutarotase facilitating the conversion of B-D-galactose to a-D-galactose since this is the active form in the pathway; next, a-D-galactose is phosphorylated by galactokinase to galactose 1-phosphate; in the third step, D-galactose-1-phosphate uridylyltransferase converts galactose 1-phosphate to UDP-galactose using UDP-glucose as the uridine diphosphate source; finally, UDP-galactose 4-epimerase recycles the UDP-galactose to UDP-glucose for the transferase reaction; and additionally. phosphoglucomutase converts the D-glucose 1-phosphate to D-glucose 6-phosphate. An illustration of the Weimberg pathway is provided in FIGS. 3 and S1 in the Appendices (filed in U.S. Provisional Patent Application Ser. Nos. 63/168,687, filed on Mar. 31, 2021). In some embodiments, the Weimberg pathway comprises, or consists essentially of, or yet further consists of one or more of the following steps: Galactose is transported or diffused from the extracellular space to the periplasm, further transported to cytosol (e.g., by gtsABCD), converted to galactose-1-P (i.e., Gal1P) (e.g., by galM, or galK or both), further to glucose-I-P (i.e., G1P) (e.g., by galT with the help of uridine diphosphate-glucose, i.e., UDP-Glc), and then to glucose-6-phosphate (i.e., G6P).

Pseudomonas putida KT2440 has been widely studied for its utilization as a microbial platform in biorefinery processes due to its tolerance to various stresses and the ability to grow on biomass-abundant aromatics (e.g., coumaric acid) (Nikel (2018), Belda (2016), Nogales (2020)). Because conventional microbial hosts such as Escherichia coli are incapable of utilizing such aromatics, the use of KT2440 is expected to improve cost effectiveness of bioprocesses by achieving whole-conversion of biomass-derivable carbon sources. In this regard, so far, various studies have been conducted to produce various biochemicals using KT2440 (Nikel (2018), Bentley (2020)). Despite these great advantages, one drawback in the use of KT2440 is that it cannot metabolize several sugars (e.g., xylose, galactose) obtainable from biomass (Lim (2013), Isikgor (2015)). While few Pseudomonas species are known to naturally have enzymes to catabolize xylose and galactose (Liu (2015), Köhler (2015), Buckel (1981)), KT2440 lacks the utilization pathway of these sugars, requiring its engineering for their efficient conversion into biochemicals. To overcome these limitations, an ALE approach was applied to engineered KT2440 strains for efficient utilization of two biomass abundant sugars, xylose and galactose. Initially, we obtained engineered KT2440 strains in which xylD from C. cresentus or galETKM from E. coli were integrated into the chromosome to construct the Weimberg pathway for xylose utilization and the Leloir pathway for galactose utilization. Then, we evolved the resulting strains in minimal media supplemented with xylose or galactose. While the initial versions grew poorly or did not grow at all, with the ALE approach, we successfully obtained evolved clones that grow on xylose or galactose with higher growth rates (0.25 h⁻¹ on xylose or 0.52 h⁻¹ on galactose). Whole-genome and transcriptome sequencing of evolved isolates revealed key mutational mechanisms that improved sugar utilization. We validated the critical roles of the introduced heterologous genes and commonly mutated genes (kguT and gtsABCD) by deleting them in evolved clones. Finally, we also confirmed their capability to serve as a platform by demonstrating efficient production of indigoidine, a naturally-found blue pigment (Wehrs (2019)).

To enable xylose and galactose utilization, KT2440 has previously been engineered by the heterologous introduction of known sugar utilization pathways. Specifically, the xylose isomerase pathway has been constructed by the expression of xylose isomerase (XylA) and xylulokinase (XylB) from Escherichia coli (Meijnen (2008), Le Meur (2012), Dvořák (2018), Wang (2019)). Additionally, the Weimberg pathway (i.e., xylose oxidative pathway) has been constructed by the expression of xylonate dehydratase (XylD) from Caulobacter crescentus (Meijnen (2009), Bator (2019)). Enabling galactose utilization has been less studied compared to xylose metabolism; two studies reported the construction of De Ley-Doudoroff (DLD) pathway (i.e., galactose oxidative pathway) (Peabody (2019)) or Leloir pathway (Banerjee (2020)) by the expression of DgoKAD, galactonate catabolic enzymes, from P. fluorescens SBW2 or GalETKM, galactose operon, from Escherichia coli K-12 MG1655, respectively. While it was shown that KT2440 can be engineered to utilize xylose and galactose, several limitations remain. We currently have insufficient understanding of which endogenous genes are involved in the heterologous sugar catabolism. Furthermore, initial studies have expressed heterologous genes using plasmids, which is not ideal and preferable for industry-scale cultivations, as plasmid-based expression increases genotypic or phenotypic instability due to its heterogeneous nature (Kang (2018), Elmore (2020)). The activation of non-native sugar utilization pathways with chromosomal expression has been demonstrated for only the xylose isomerase pathway (Elmore (2020)) and De Ley-Doudoroff pathway (Peabody (2019)). Genome-based engineering often resulted in unsatisfactory cell growth (i.e., slow growth rate or long lag phase) which makes their practical deployments challenging. Given each utilization pathway generates different intermediates and biochemical yields (Bator (2019)), further studies to construct less-explored pathways in KT2440 are warranted. As a summary, compared to strains that were reported in the previous studies, we can provide cells with higher xylose or galactose utilization capability even without using plasmids.

The evolved strains show high growth rates and sugar uptake rates on xylose or galactose supplemented medium. Therefore, these strains can be used as a host in diverse microorganism-based bioprocesses for the efficient conversion of biomass containing xylose or galactose.

In some embodiments, the Pseudomonas cell is P. putida KT2440 (Biorxiv DOI number: 10.1101/139121), which is publicly available.

The amino acid sequence of the Caulobacter crescentus xylD gene product is as follows:

```
(SEQ ID NO: 1)
                10 20 30 40
MSNRTPRRFR SRDWFDNPDH IDMTALYLER FMNYGITPEE
                50 60 70 80
LRSGKPIIGI AQTGSDISPC NRIHLDLVQR VRDGIRDAGG
                90 100 110 120
IPMEFPVHPI FENCRRPTAA LDRNLSYLGL VETLHGYPID
              130 140 150 160
AVVLTTGCDK TTPAGIMAAT TVNIPAIVLS GGPMLDGWHE
              170 180 190 200
NELVGSGTVI WRSRRKLAAG EITEEEFIDR AASSAPSAGH
              210 220 230 240
CNTMGTASTM NAVAEALGLS LTGCAAIPAP YRERGQMAYK
              250 260 270 280
TGQRIVDLAY DDVKPLDILT KQAFENAIAL VAAAGGSTNA
              290 300 310 320
QPHIVAMARH AGVEITADDW RAAYDIPLIV NMQPAGKYLG
              330 340 350 360
ERFHRAGGAP AVLWELLQQG RLHGDVLTVT GKTMSENLQG
              370 380 390 400
RETSDREVIF PYHEPLAEKA GFLVLKGNLF DFAIMKSSVI
              410 420 430 440
GEEFRKRYLS QPGQEGVFEA RAIVFDGSDD YHKRINDPAL
              450 460 470 480
EIDERCILVI RGAGPIGWPG SAEVVNMQPP DHLLKKGIMS
              490 500 510 520
LPTLGDGRQS GTADSPSILN ASPESAIGGG LSWLRTGDTI
              530 540 550 560
RIDLNTGRCD ALVDEATIAA RKQDGIPAVP ATMTPWQEIY
              570 580 590
RAHASQLDTG GVLEFAVKYQ DLAAKLPRHN H
```

The amino acid sequence of the Escherichia coli galE gene product is as follows:

```
(SEQ ID NO: 2)
                10 20 30 40
MRVLVTGGSG YIGSHTCVQL LQNGHDVIIL DNLCNSKRSV
                50 60 70 80
LPVIERLGGK HPTFVEGDIR NEALMTEILH DHAIDTVIHF
                90 100 110 120
AGLKAVGESV QKPLEYYDNN VNGTLRLISA MRAANVKNFI
              130 140 150 160
FSSSATVYGD QPKIPYVESF PTGTPQSPYG KSKLMVEQIL
              170 180 190 200
TDLQKAQPDW SIALLRYFNP VGAHPSGDMG EDPQGIPNNL
              210 220 230 240
MPYIAQVAVG RRDSLAIFGN DYPTEDGTGV RDYIHVMDLA
              250 260 270 280
DGHVVAMEKL ANKPGVHIYN LGAGVGNSVL DVVNAFSKAC
              290 300 310 320
GKPVNYHFAP RREGDLPAYW ADASKADREL NWRVTRTLDE
              330
MAQDTWHWQS RHPQGYPD
```

The amino acid sequence of the Escherichia coli galT gene product is as follows:

```
(SEQ ID NO: 3)
                10 20 30 40
MTQFNPVDHP HRRYNPLTGQ WILVSPHRAK RPWQGAQETP
                50 60 70 80
AKQVLPAHDP DCFLCAGNVR VTGDKNPDYT GTYVFTNDFA
                90 100 110 120
ALMSDTPDAP ESHDPLMRCQ SARGTSRVIC FSPDHSKTLP
              130 140 150 160
ELSVAALTEI VKTWQEQTAE LGKTYPWVQV FENKGAAMGC
              170 180 190 200
SNPHPHGQIW ANSFLPNEAE REDRLQKEYF AEQKSPMLVD
              210 220 230 240
YVQRELADGS RTVVETEHWL AVVPYWAAWP FETLLLPKAH
              250 260 270 280
VLRITDLTDA QRSDLALALK KLTSRYDNLF QCSFPYSMGW
              290 300 310 320
HGAPENGEEN QHWQLHAHFY PPLLRSATVR KFMVGYEMLA
              330 340
ETQRDLTAEQ AAERLRAVSD IHFRESGV
```

The amino acid sequence of the Escherichia coli galK gene product is as follows:

```
(SEQ ID NO: 4)
                10 20 30 40
MSLKEKTQSL FANAFGYPAT HTIQAPGRVN LIGEHTDYND
                50 60 70 80
GFVLPCAIDY QTVISCAPRD DRKVRVMAAD YENQLDEFSL
                90 100 110 120
DAPIVAHENY QWANYVRGVV KHLQLRNNSF GGVDMVISGN
              130 140 150 160
VPQGAGLSSS ASLEVAVGTV LQQLYHLPLD GAQIALNGQE
              170 180 190 200
AENQFVGCNC GIMDQLISAL GKKDHALLID CRSLGTKAVS
              210 220 230 240
MPKGVAVVII NSNFKRTLVG SEYNTRREQC ETGARFFQQP
              250 260 270 280
ALRDVTIEEF NAVAHELDPI VAKRVRHILT ENARTVEAAS
              290 300 310 320
ALEQGDLKRM GELMAESHAS MRDDFEITVP QIDTLVEIVK
              330 340 350 360
AVIGDKGGVR MTGGGFGGCI VALIPEELVP AVQQAVAEQY
              370 380
EAKTGIKETF YVCKPSQGAG QC
```

The amino acid sequence of the Escherichia coli galM gene product is as follows:

```
(SEQ ID NO: 5)
                10 20 30 40
MLNETPALAP DGQPYRLLTL RNNAGMVVTL MDWGATLLSA
                50 60 70 80
RIPLSDGSVR EALLGCASPE CYQDQAAFLG ASIGRYANRI
                90 100 110 120
ANSRYTFDGE TVTLSPSQGV NQLHGGPEGF DKRRWQIVNQ
              130 140 150 160
NDRQVLFALS SDDGDQGFPG NLGATVQYRL TDDNRISITY
              170 180 190 200
RATVDKPCPV NMTNHVYFNL DGEQSDVRNH KLQILADEYL
              210 220 230 240
PVDEGGIPHD GLKSVAGTSF DFRSAKIIAS EFLADDDQRK
              250 260 270 280
VKGYDHAFLL QAKGDGKKVA AHVWSADEKL QLKVYTTAPA
              290 300 310 320
LQFYSGNFLG GTPSRGTEPY ADWQGLALES EFLPDSPNHP
              330 340
EWPQPDCFLR PGEEYSSLTE YQFIAE
```

The nucleotide sequence of the xylD expression cassette:

(SEQ ID NO: 6)
Aggctgtctcgtctcgtctc tttacggctagctcagtcctaggtacaatgctagc aacaacagcttagaaggaggtcaat atg
-------------------- +++++++++++++++++++++++++++++++++++ ^^^^^^^^^^^^^^^^^^^^^^^^^ start
aggtccgccttgtctaaccgcacgccccgccggttccggtcccgcgattggttcgataaccccgaccatatcgacatgaccgcgctctatct
ggagcgcttcatgaactacgggatcacgccggaggagctgcgcagcggcaagccgatcatcggcatcgcccagaccggcagcgacat
ctcgccctgcaaccgcatccacctggacctggtccagcgggtgcgggacgggatccgcgacgccgggggcatccccatggagttcccg
gtccatccgatcttcgagaactgccgtcgcccgacggcggcgctggaccggaacctctcgtacctgggtctcgtcgagaccctgcacggc
tatccgatcgacgccgtggttctgaccaccggctgcgacaagaccaccccggccgggatcatggccgccaccacggtcaatatcccggc
catcgtgctgtcgggcggcccgatgctggacggctggcacgagaacgagctcgtgggctcgggcaccgtgatctggcgctcgcgccgc
aagctggcggccggcgagatcaccgaggaagagttcatcgaccgcgccgccagctcggcgccgtcggcgggccactgcaacaccatg
ggcacggcctcgaccatgaacgccgtggccgaggcgctgggcctgtcgctgaccggctgcgcggccatccccgccccctaccgcgag
cgcggccagatggcctacaagaccggccagcgcatcgtcgatctggcctatgacgacgtcaaaccgctcgacatcctgaccaagcaagc
cttcgagaacgccatcgccctggtggcggcggccggcggctcgaccaacgcccagccgcacatcgtggccatggcccgtcacgccgg
cgtcgagatcaccgccgacgactggcgcgcggcctatgacatcccgctgatcgtcaacatgcagccggccggcaagtatctgggcgagc
gcttccaccgagccggcggcgcgccggcggtgctgtgggagctgttgcagcaaggccgcctgcacggcgacgtgctgaccgtcaccg
gcaagacgatgagcgagaacctgcaaggccgcgaaaccagcgaccgcgaggtgatcttcccgtaccacgagccgctggccgagaagg
ccgggttcctggttctcaagggcaacctcttcgacttcgcgatcatgaagtccagcgtgatcggcgaggagttccgcaagcgctacctgtcg
cagcccggccaggaaggcgtgttcgaagcccgcgccatcgtgttcgacggctcggacgactatcacaagcggatcaacgatccggccct
ggagatcgacgagcgctgcatcctggtgatccgcggcgcgggtccgatcggctggcccggctcggccgaggtcgtcaacatgcagccg
ccggatcaccttctgaagaaggggatcatgagcctgcccaccctgggcgatggccgtcagtcgggcaccgccgacagcccctcgatcct
gaacgcctcgcccgaaagcgcgatcggcggcggcctgtcgtggctgcgcaccggcgacaccatccgcatcgacctcaacaccggccg
ctgcgacgccctggtcgacgaggcgacgatcgccgcgcgcaagcaggacggcatcccggcggttcccgccaccatgacgccctggca
ggaaatctaccgcgcccacgccagtcagctcgacaccggcggcgtgctggagttcgcggtcaagtaccaggacctggcggccaagctg
ccccgccacaaccac
tga gctgggagttcgtagacgga cgcaaaaaaccccgcttcggcggggttttttcgc
stop -------------------- **********************************

Code: “---” indicates the insulating sequences (Torella et al., 2014); “+++” indicates the BBa_J23110 promoter; “^{AAA}” indicates the 5′-untranslated region; “start” indicates the start codon; “stop” indicates the stop codon; “***” indicates the BBa_B1002_terminator.

In some embodiments, the Pseudomonas cell comprises a nucleic acid encoding the indicated gene operatively linked to a promoter capable of expressing the gene in the Pseudomonas cell. In some embodiments, the encoding of the gene in the nucleic acid is codon optimized to the Pseudomonas cell. In some embodiments, the nucleic acid is stably integrated into one or more chromosomes of the Pseudomonas cell.

The present invention provides for a method for a Pseudomonas cell producing indigoidine, comprising (a) providing a Pseudomonas cell of the present invention, (b) culturing or growing the Pseudomonas cell in a suitable culture or medium such that indigoidine is produced, and (c) optionally extracting or separating the indigoidine from the rest of the culture or medium, and/or Pseudomonas cell.

In some embodiments, the providing step (a) comprises introducing a nucleic acid encoding the indicated gene(s) operatively linked to a promoter capable of expressing the indicated gene(s) in the Pseudomonas cell into the Pseudomonas cell.

In some embodiments, the culturing or growing step (b) comprises the Pseudomonas cell growing by respiratory cell growth. In some embodiments, the culturing or growing step (b) takes place in a batch process or a fed-batch process, such as a high-gravity fed-batch process. In some embodiments, the culture or medium comprises hydrolysates derived or obtained from a biomass, such as a lignocellulosic biomass. In some embodiments, the culture or medium comprises one or more carbon sources, such as a sugar, such as a xylose and/or galactose, or a mixture thereof. In some embodiments, the carbon source is fermentable. In some embodiments, the carbon source is non-fermentable.

The present invention provides for a method for constructing a Pseudomonas cell of the present invention, comprising (a) introducing a nucleic acid encoding the indicated gene(s) operatively linked to a promoter capable of expressing the indicated gene(s) in the Pseudomonas cell into the Pseudomonas cell, and optionally introducing one of the indicated mutation(s) of the genes described herein in the chromosome of the Pseudomonas cell.

One can modify the expression of a gene encoding any of the enzymes taught herein by a variety of methods in accordance with the methods of the invention. Those skilled in the art would recognize that increasing gene copy number, ribosome binding site strength, promoter strength, and various transcriptional regulators can be employed to alter an enzyme expression level.

In one aspect, provided herein is an engineered Pseudomonas bacterium comprising at least one heterologous protein(s) (i.e., a protein heterologous to a Pseudomonas bacterium, for example, a wild type Pseudomonas bacterium or a wild type Pseudomonas putida KT2440) of the Weimberg pathway, and at least one mutation(s) in a chromosome of the bacterium.

In some embodiments, the at least one heterologous protein(s) comprises xylonate dehydratase. In some embodiments, the at least one heterologous protein(s) comprises, or essentially consists of, or yet further consists of xylonate dehydratase of Caulobacter crescentus.

In some embodiments, the xylonate dehydratase is of a species selected from Caulobacter crescentus. Caulobacter vibrioides, or Halomonas elongate. In some embodiments, the xylonate dehydratase is encoded by a heterologous xylD gene. In some embodiments, the bacterium is Pseudomonas putida KT2440.

In some embodiments, the bacterium catabolizes xylose.

In some embodiments, the at least one mutation(s) comprises, or consists essentially of, or yet further consists of a mutation in a gene(s) or intergenic region(s) selected from the group of: betT-II; cysQ; mmsA-I, PP_16SD; ettA; phnC; PP_1028; xcpUlxcpV; PP_1104; galP-1, PP_1174; yhdX; yhdY; PP_1475; mutS; gacS; apeB; PP_1886; PP_1980, dusC; PP_2222; PP_2277; PP_2287; PP_2411; sad-I, PP_2489; PP_2628; PP_2851; PP_2855, PP_2856; PP_2962, PP_2964; dnaEB; kguT; ptxS; PP_3475; PP_3573; PP_3645; creA, katG; ribAB-II; PP_4085; uvrY; PP_4173; fliI; ooxB; ftsH; PP_4824; parC; PP_4926; PP_4950; PP_4983; PP_5043; betT-I; PP_5167, cysA; PP_5729, rpmG; the intergenic region between galP-1 and PP_1174 (noted as galP-I/PP_1174); or any other gene or intergenic region as disclosed herein.

In some embodiments, the at least one mutation(s) comprises, or consists essentially of, or yet further consists of a mutation in a gene(s) or intergenic region(s) selected from the group of: galP-I, PP_1174; gacS; kguT; ptxS; PP_4173; or ftsH.

In some embodiments, the at least one mutation(s) comprises, or consists essentially of, or yet further consists of a mutation disclosed in Sheet 3 of Supplementary Data 3. In some embodiments, the at least one mutation(s) comprises, or essentially consists of, or yet further consists of one or more of gain-of-function mutation(s) in genes selected from the group of: kguE, kguK, kguT, ptxD, gcd or another gene a gene product of which transports xylonate. In some embodiments, the at least one mutation(s) comprises, or essentially consists of, or yet further consists of one or more of loss-of-function mutation(s) in ptxS.

In some embodiments, the mutations and their positions as disclosed herein are noted and numbered based on the P. putida KT2440 genome disclosed in Lim et al., 2020. doi:10.1039/D0GC01663B, which is incorporated herein by reference in its entirety. In other embodiments, the mutations and their positions as disclosed herein are noted and numbered based on NCBI Reference Sequence: NC_002947.4, NCBI Reference Sequence: NC_021505.1, or GenBank: AE015451.2, each of which is enclosed herein by its entirety. A bacterial genome is a double stranded DNA comprising, or consisting essentially of, or yet further consisting of a plus strand and minus strand, which reverse complementary to each other. Accordingly, in some embodiments, the reference genome provides a sequence, which is referred to herein as a plus strand. In further embodiments, the nucleotide residue prior to a mutation as used herein is the one in the plus strand unless specified. In some embodiments, a mutation as disclosed herein is determined as a mutation if different compared to a wild type, for example, the P. putida KT2440 stain as disclosed in Lim et al., 2020. doi:10.1039/D0GC01663B. NCBI Reference Sequence: NC_002947.4. NCBI Reference Sequence: NC_021505.1, or GenBank: AE015451.2.

As it would be understood by one of skill in the art, the gene mutation as disclosed herein may be substituted with another gene mutation that encodes the same mutated protein.

In some embodiments, sequences of the genes and proteins as disclosed herein is available at ncbi.nlm.nih.gov and uniprot.org, each of which is enclosed herein by its entirety.

In some embodiments, the at least one mutation(s) comprises, or consists essentially of, or yet further consists of a mutation at position 283582, optionally the third nucleotide residue in the codon encoding S344 of the protein, from C optionally to T in the gene of betT-II.

In some embodiments, the at least one mutation(s) comprises, or consists essentially of, or yet further consists of a mutation at position 315243, optionally the second nucleotide residue in the codon encoding W19 of the protein, from G optionally to T in the gene of cysQ. In further embodiments, the mutated gene encodes a cysQ protein comprising a mutation of W19L.

In some embodiments, the at least one mutation(s) comprises, or consists essentially of, or yet further consists of a mutation at position 697097, from A optionally to G in the intergenic region between the genes of mmsA-I and PP_16SD, optionally at the intergenic position of (+21/−730).

In some embodiments, the at least one mutation(s) comprises, or consists essentially of, or yet further consists of a mutation at position 697103, from T optionally to G in the intergenic region between the genes of mmsA-I and PP_16SD, optionally at the intergenic position of (+27/−724).

In some embodiments, the at least one mutation(s) comprises, or consists essentially of, or yet further consists of a mutation at position 787253, optionally the second nucleotide residue in the codon encoding A123 of the protein, from C optionally to T in the gene of ettA. In further embodiments, the mutated gene encodes a protein comprising a mutation of A123V.

In some embodiments, the at least one mutation(s) comprises, or consists essentially of, or yet further consists of a mutation at position 963645, optionally the second nucleotide residue in the codon encoding A84 of the protein, from C optionally to T in the gene of phnC. In further embodiments, the mutated gene encodes a protein comprising a mutation of A84V.

In some embodiments, the at least one mutation(s) comprises, or consists essentially of, or yet further consists of a mutation at position 1173697, optionally the second nucleotide residue in the codon encoding L112 of the protein, from T optionally to C in the plus strand of the bacterium DNA (i.e., A optionally to G in the protein coding strand), in the gene of PP_1028. In further embodiments, the mutated gene encodes a protein comprising a mutation of L112P.

In some embodiments, the at least one mutation(s) comprises, or consists essentially of, or yet further consists of a mutation at position 1201211 from C optionally to T. In one embodiment, the mutation at position 1201211 is in the gene of xcpU. In further embodiments, the mutation at position 1201211 is the third nucleotide residue in the codon encoding S134 of the xcpU protein. In another embodiments, the mutation at position 1201211 is in the gene of xcpV. In further embodiments, the mutation at position 1201211 is the first nucleotide residue in the codon encoding P5 of the xcpV protein. In yet further embodiments, the mutated gene encodes an xcpV protein comprising a mutation of P5S.

In some embodiments, the at least one mutation(s) comprises, or consists essentially of, or yet further consists of a mutation at position 1261817, optionally an insertion of a nucleotide residue G. In further embodiments, the insertion is in the gene of PP_1104. In yet further embodiments, the insertion is at the 824th nucleotide (nt) residue of the 1116-nt-long coding sequence of PP_1104, optionally counted from the 5′ of the coding sequence.

In some embodiments, the at least one mutation(s) comprises, or consists essentially of, or yet further consists of a mutation at position 1348783 from C optionally to T in the intergenic region between the genes of galP-I and PP_1174, optionally at the intergenic position of (−243/+511).

In some embodiments, the at least one mutation(s) comprises, or consists essentially of, or yet further consists of a mutation at position 1348789 from A optionally to G in the intergenic region between the genes of galP-I and PP_1174, optionally at the intergenic position of (−249/+505).

In some embodiments, the at least one mutation(s) comprises, or consists essentially of, or yet further consists of a mutation at position 1484303, optionally the second nucleotide residue in the codon encoding V125 of the protein, from T optionally to G in the gene of yhdX. In further embodiments, the mutated gene encodes a protein comprising a mutation of V125G.

In some embodiments, the at least one mutation(s) comprises, or consists essentially of, or yet further consists of a mutation at position 1485547, optionally an insertion of a nucleotide residue G. In further embodiments, the insertion is in the gene of yhdY. In yet further embodiments, the insertion is at the 429th nucleotide (nt) residue of the 1098-nt-long coding sequence of yhdY, optionally counted from the 5′ of the coding sequence.

In some embodiments, the at least one mutation(s) comprises, or consists essentially of, or yet further consists of a mutation at position 168(0017, optionally the first nucleotide residue in the codon encoding T276 of the protein, from T optionally to G in the plus strand of the bacterial DNA (i.e., from A optionally to C in the protein coding strand), in the gene of PP_1475. In further embodiments, the mutated gene encodes a protein comprising a mutation of T276P.

In some embodiments, the at least one mutation(s) comprises, or consists essentially of, or yet further consists of a mutation at position 1825254, optionally the third nucleotide residue in the codon encoding Y14 of the protein, from G optionally to T in the plus strand of the bacterium DNA (i.e., C optionally to A in the protein coding strand), in the gene of mutS. In further embodiments, the mutated gene encodes a truncated protein terminated at Y14, i.e., only consisting of the first 13 amino acid residues.

In some embodiments, the at least one mutation(s) comprises, or consists essentially of, or yet further consists of a mutation at position 1842929, optionally the second nucleotide residue in the codon encoding P636 of the protein, from G optionally to T in the plus strand of the bacterial DNA (i.e., C optionally to A in the protein coding strand), in the gene of gacS. In further embodiments, the mutated gene encodes a protein comprising a mutation of P636Q.

In some embodiments, the at least one mutation(s) comprises, or consists essentially of, or yet further consists of a mutation at position 1843229, optionally an insertion of GCGCCAGTTCGTGGC (SEQ ID NO: 7). In further embodiments, the insertion is in the gene of gacS. In yet further embodiments, the insertion is at the 1607th nucleotide (nt) residue of the 2754-nt-long coding sequence of gacS, optionally counted from the 5′ of the coding sequence.

In some embodiments, the at least one mutation(s) comprises, or consists essentially of, or yet further consists of a mutation at position 1844030, optionally the second nucleotide residue in the codon encoding N269 of the protein, from G optionally to T in the plus strand of the bacterial DNA (i.e., from C optionally to A in the protein coding strand), in the gene of gacS. In further embodiments, the mutated gene encodes a protein comprising a mutation of N269S.

In some embodiments, the at least one mutation(s) comprises, or consists essentially of, or yet further consists of a mutation at position 1930434, optionally a deletion in the gene of apeB. In further embodiments, the deletion comprises, or consists essentially of, or further consists of deletions at position 1930434 and in the downstream 62 nucleotide residues.

In some embodiments, the at least one mutation(s) comprises, or consists essentially of, or yet further consists of a mutation at position 2123260, optionally the third nucleotide residue in the codon encoding R41 of the protein, from T optionally to C in the gene of PP_1886.

In some embodiments, the at least one mutation(s) comprises, or consists essentially of, or yet further consists of a mutation at position 2246373, from C optionally to G in the intergenic region between the genes of PP_1980 and dusC, optionally at the intergenic position of (+5/−47).

In some embodiments, the at least one mutation(s) comprises, or consists essentially of, or yet further consists of a mutation at position 2533751, optionally the second nucleotide residue in the codon encoding S14 of the protein, from C optionally to T in the gene of PP_2222. In further embodiments, the mutated gene encodes a protein comprising a mutation of S14L.

In some embodiments, the at least one mutation(s) comprises, or consists essentially of, or yet further consists of a mutation at position 2601596, optionally the first nucleotide residue in the codon encoding T324A of the protein, from A optionally to G in the gene of PP_2277. In further embodiments, the mutated gene encodes a protein comprising a mutation of T324A.

In some embodiments, the at least one mutation(s) comprises, or consists essentially of, or yet further consists of a mutation at position 2613426, optionally the first nucleotide residue in the codon encoding T134 of the protein, from A optionally to G in the gene of PP_2287. In further embodiments, the mutated gene encodes a protein comprising a mutation of T134A.

In some embodiments, the at least one mutation(s) comprises, or consists essentially of, or yet further consists of a mutation at position 2758975, optionally the third nucleotide residue in the codon encoding A394 of the protein, from T optionally to C in the plus strand of the bacterial DNA (i.e., from A optionally to G in the protein coding strand), in the gene of PP_2411.

In some embodiments, the at least one mutation(s) comprises, or consists essentially of, or yet further consists of a mutation at position 2839129, from C optionally to A in the intergenic region between the genes of sad-I and PP_2489, optionally at the intergenic position of (−116/+67).

In some embodiments, the at least one mutation(s) comprises, or consists essentially of, or yet further consists of a mutation at position 3009219, optionally the second nucleotide residue in the codon encoding M614 of the protein, from A optionally to G in the plus strand of the bacterial DNA (i.e., from T to C in the protein coding strand), in the gene of PP_2628. In further embodiments, the mutated gene encodes a protein comprising a mutation of M614T.

In some embodiments, the at least one mutation(s) comprises, or consists essentially of, or yet further consists of a mutation at position 3254711, optionally the third nucleotide residue in the codon encoding G232 of the protein, from C optionally to A in the plus strand of the bacterial DNA (i.e., from G to T in the protein coding strand), in the gene of PP_2851.

In some embodiments, the at least one mutation(s) comprises, or consists essentially of, or yet further consists of a mutation at position 3260681, optionally an insertion of a nucleotide residue C. In further embodiments, the insertion is in the intergenic region between the genes of PP_2855 and PP_2856, optionally at the intergenic position of (−38/+24).

In some embodiments, the at least one mutation(s) comprises, or consists essentially of, or yet further consists of a mutation at position 3368556, from T optionally to C in the intergenic region between the genes of PP_2962 and PP_2964, optionally at the intergenic position of (+257/−104).

In some embodiments, the at least one mutation(s) comprises, or consists essentially of, or yet further consists of a mutation at position 3533129, optionally the second nucleotide residue in the codon encoding L994 of the protein, from T optionally to C in the gene of dnaEB. In further embodiments, the mutated gene encodes a protein comprising a mutation of L994P.

In some embodiments, the at least one mutation(s) comprises, or consists essentially of, or yet further consists of a mutation at position 3823860, optionally a deletion in the gene of kguT. In further embodiments, the deletion comprises, or consists essentially of, or further consists of deletions at position 3823860 and in the downstream 8 nucleotide residues. In yet further embodiments the deletion comprises, or consists essentially of, or further consists of deletions of the 1281st nucleotide (nt) residue to the 1289th nt residue of the 1293-nt-long coding sequence of kguT, optionally counted from the 5′ of the coding sequence.

In some embodiments, the at least one mutation(s) comprises, or consists essentially of, or yet further consists of a mutation at position 3823901, optionally an insertion of a nucleotide residue T. In further embodiments, the insertion is in the gene of kguT. In yet further embodiments, the insertion is at the 1248th nucleotide (nt) residue of the 1293-nt-long coding sequence of kguT, optionally counted from the 5′ of the coding sequence.

In some embodiments, the at least one mutation(s) comprises, or consists essentially of, or yet further consists of a mutation at position 3824314, optionally the first nucleotide residue in the codon encoding A279 of the protein, from C optionally to G in the plus strand of the bacterial DNA (i.e., from G optionally to C in the protein coding strand), in the gene of kguT. In further embodiments, the mutated gene encodes a protein comprising a mutation of A279P.

In some embodiments, the at least one mutation(s) comprises, or consists essentially of, or yet further consists of a mutation at position 3824415, optionally the second nucleotide residue in the codon encoding 1245 of the protein, from A optionally to G in the plus strand of the bacterial DNA (i.e., from T optionally to C in the protein coding strand), in the gene of kguT. In further embodiments, the mutated gene encodes a protein comprising a mutation of 1245T.

In some embodiments, the at least one mutation(s) comprises, or consists essentially of, or yet further consists of a mutation at position 3824695, optionally the first nucleotide residue in the codon encoding L152 of the protein, from G optionally to C in the plus strand of the bacterial DNA (i.e., from C optionally to G in the protein coding strand), in the gene of kguT. In further embodiments, the mutated gene encodes a protein comprising a mutation of L152V.

In some embodiments, the at least one mutation(s) comprises, or consists essentially of, or yet further consists of a mutation at position 3824717, optionally the third nucleotide residue in the codon encoding M144 of the protein, from C optionally to T in the plus strand of the bacterial DNA (i.e., from G optionally to A in the protein coding strand), in the gene of kguT. In further embodiments, the mutated gene encodes a protein comprising a mutation of M144I.

In some embodiments, the at least one mutation(s) comprises, or consists essentially of, or yet further consists of a mutation at position 3824719, optionally the first nucleotide residue in the codon encoding M144 of the protein, from T optionally to C in the plus strand of the bacterial DNA (i.e., from A optionally to G in the protein coding strand), in the gene of kguT. In further embodiments, the mutated gene encodes a protein comprising a mutation of M144V.

In some embodiments, the at least one mutation(s) comprises, or consists essentially of, or yet further consists of a mutation at position 3824800, optionally the first nucleotide residue in the codon encoding A 17 of the protein, from C optionally to A in the plus strand of the bacterial DNA (i.e., from G optionally to T in the protein coding strand), in the gene of kguT. In further embodiments, the mutated gene encodes a protein comprising a mutation of A117S.

In some embodiments, the at least one mutation(s) comprises, or consists essentially of, or yet further consists of a mutation at position 3825009, optionally the second nucleotide residue in the codon encoding T47 of the protein, from G optionally to T in the plus strand of the bacterial DNA (i.e., from C optionally to A in the protein coding strand), in the gene of kguT. In further embodiments, the mutated gene encodes a protein comprising a mutation of T47N.

In some embodiments, the at least one mutation(s) comprises, or consists essentially of, or yet further consists of a mutation at position 3827189, optionally a deletion in the gene of ptxS. In further embodiments, the deletion comprises, or consists essentially of, or further consists of deletions at position 3827189 and in the downstream 437 nucleotide residues. In yet further embodiments the deletion comprises, or consists essentially of, or further consists of deletions of the 385th nucleotide (nt) residue to the 822nd nt residue of the 1020-nt-long coding sequence of ptxS, optionally counted from the 5′ of the coding sequence.

In some embodiments, the at least one mutation(s) comprises, or consists essentially of, or yet further consists of a mutation at position 3827335, optionally a deletion in the gene of ptxS. In further embodiments, the deletion comprises, or consists essentially of, or further consists of deletions at position 3827335 and in the downstream 50 nucleotide residues. In yet further embodiments the deletion comprises, or consists essentially of, or further consists of deletions of the 626th nucleotide (nt) residue to the 676th nt residue of the 1020-nt-long coding sequence of ptxS, optionally counted from the 5′ of the coding sequence.

In some embodiments, the at least one mutation(s) comprises, or consists essentially of, or yet further consists of a mutation at position 3827923, optionally the first nucleotide residue in the codon encoding R30 of the protein, from G optionally to T in the plus strand of the bacterial DNA (i.e., from C optionally to A in the protein coding strand), in the gene of ptxS. In further embodiments, the mutated gene encodes a protein comprising a mutation of R30S.

In some embodiments, the at least one mutation(s) comprises, or consists essentially of, or yet further consists of a mutation at position 3827925, optionally the second nucleotide residue in the codon encoding S29 of the protein, from G optionally to A in the plus strand of the bacterial DNA (i.e., from C optionally to T in the protein coding strand), in the gene of ptxS. In further embodiments, the mutated gene encodes a protein comprising a mutation of S29F.

In some embodiments, the at least one mutation(s) comprises, or consists essentially of, or yet further consists of a mutation at position 3827929, optionally the first nucleotide residue in the codon encoding V28 of the protein, from C optionally to A in the plus strand of the bacterial DNA (i.e., from G optionally to T in the protein coding strand), in the gene of ptxS. In further embodiments, the mutated gene encodes a protein comprising a mutation of V28F.

In some embodiments, the at least one mutation(s) comprises, or consists essentially of, or yet further consists of a mutation at position 3942797, optionally the third nucleotide residue in the codon encoding S123 of the protein, from G optionally to A in the plus strand of the bacterial DNA (i.e., from C optionally to T in the protein coding strand), in the gene of PP_3475.

In some embodiments, the at least one mutation(s) comprises, or consists essentially of, or yet further consists of a mutation at position 4056572, optionally an insertion of a nucleotide residue G. In further embodiments, the insertion is in the gene of PP_3573. In yet further embodiments, the insertion is at the 1180th nucleotide (nt) residue of the 1320-nt-long coding sequence of PP_3573, optionally counted from the 5′ of the coding sequence.

In some embodiments, the at least one mutation(s) comprises, or consists essentially of, or yet further consists of a mutation at position 4143477, optionally the third nucleotide residue in the codon encoding R 158 of the protein, from T optionally to C in the gene of PP_3645.

In some embodiments, the at least one mutation(s) comprises, or consists essentially of, or yet further consists of a mutation at position 4169591, optionally an insertion of a nucleotide residue C. In further embodiments, the insertion is in the intergenic region between the genes of creA and katG, optionally at the intergenic position of −150/+12).

In some embodiments, the at least one mutation(s) comprises, or consists essentially of, or yet further consists of a mutation at position 4341656, optionally the second nucleotide residue in the codon encoding E266 of the protein, from A optionally to G in the gene of ribAB-

In further embodiments, the mutated gene encodes a protein comprising a mutation of E266G.

In some embodiments, the at least one mutation(s) comprises, or consists essentially of, or yet further consists of a mutation at position 4618760, optionally the first nucleotide residue in the codon encoding G1055 of the protein, from G optionally to A in the gene of PP_4085. In further embodiments, the mutated gene encodes a protein comprising a mutation of G1055S.

In some embodiments, the at least one mutation(s) comprises, or consists essentially of, or yet further consists of a mutation at position 4637297, optionally the first nucleotide residue in the codon encoding S143 of the protein, from A optionally to G in the plus strand of the bacterial DNA (i.e., from T optionally to C in the protein coding strand), in the gene of uvrY. In further embodiments, the mutated gene encodes a protein comprising a mutation of S143P.

In some embodiments, the at least one mutation(s) comprises, or consists essentially of, or yet further consists of a mutation at position 4717084, optionally the second nucleotide residue in the codon encoding F116 of the protein, from T optionally to C in the gene of PP_4173. In further embodiments, the mutated gene encodes a protein comprising a mutation of F116S.

In some embodiments, the at least one mutation(s) comprises, or consists essentially of, or yet further consists of a mutation at position 4717316, optionally an insertion of CTGAGCCTGGCC (SEQ ID NO: 8). In further embodiments, the insertion is in the gene of PP_4173. In yet further embodiments, the insertion is at the 579th nucleotide (nt) residue of the 1932-nt-long coding sequence of PP_4173, optionally counted from the 5′ of the coding sequence.

In some embodiments, the at least one mutation(s) comprises, or consists essentially of, or yet further consists of a mutation at position 4717498, optionally the second nucleotide residue in the codon encoding A254 of the protein, from C optionally to G in the gene of PP_4173. In further embodiments, the mutated gene encodes a protein comprising a mutation of A254G.

In some embodiments, the at least one mutation(s) comprises, or consists essentially of, or yet further consists of a mutation at position 4958434, optionally an insertion of a nucleotide residue C. In further embodiments, the insertion is in the gene of fliI. In yet further embodiments, the insertion is at the 288th nucleotide (nt) residue of the 1374-nt-long coding sequence of fliI, optionally counted from the 5′ of the coding sequence.

In some embodiments, the at least one mutation(s) comprises, or consists essentially of, or yet further consists of a mutation at position 5060944, optionally an insertion of a nucleotide residue C. In further embodiments, the insertion is in the gene of ooxB. In yet further embodiments, the insertion is at the 279th nucleotide (nt) residue of the 1128-nt-long coding sequence of ooxB, optionally counted from the 5′ of the coding sequence.

In some embodiments, the at least one mutation(s) comprises, or consists essentially of, or yet further consists of a mutation at position 5367426, optionally the second nucleotide residue in the codon encoding A367 of the protein, from G optionally to A in the plus strand of the bacterial DNA (i.e., from C optionally to T in the protein coding strand), in the gene of ftsH. In further embodiments, the mutated gene encodes a protein comprising a mutation of A367V.

In some embodiments, the at least one mutation(s) comprises, or consists essentially of, or yet further consists of a mutation at position 5367766, optionally the first nucleotide residue in the codon encoding F254 of the protein, from A optionally to G in the plus strand of the bacterial DNA (i.e., from T optionally to C in the protein coding strand), in the gene of ftsH. In further embodiments, the mutated gene encodes a protein comprising a mutation of F254L.

In some embodiments, the at least one mutation(s) comprises, or consists essentially of, or yet further consists of a mutation at position 5367931, optionally the first nucleotide residue in the codon encoding P199 of the protein, from G optionally to A in the plus strand of the bacterial DNA (i.e., from C optionally to T in the protein coding strand), in the gene of ftsH. In further embodiments, the mutated gene encodes a protein comprising a mutation of P199S.

In some embodiments, the at least one mutation(s) comprises, or consists essentially of, or yet further consists of a mutation at position 5489180, optionally the second nucleotide residue in the codon encoding G180 of the protein, from G optionally to A in the gene of PP_4824. In further embodiments, the mutated gene encodes a protein comprising a mutation of G180D.

In some embodiments, the at least one mutation(s) comprises, or consists essentially of, or yet further consists of a mutation at position 5585751, optionally the first nucleotide residue in the codon encoding A652 of the protein, from C optionally to T in the plus strand of the bacterial DNA (i.e., from G optionally to A in the protein coding strand), in the gene of parC. In further embodiments, the mutated gene encodes a protein comprising a mutation of A652T.

In some embodiments, the at least one mutation(s) comprises, or consists essentially of, or yet further consists of a mutation at position 5607194, the third nucleotide residue in the codon encoding A242 of the protein, from C optionally to T in the plus strand of the bacterial DNA (i.e., from G optionally to A in the protein coding strand), in the gene of PP_4926.

In some embodiments, the at least one mutation(s) comprises, or consists essentially of, or yet further consists of a mutation at position 5641230, optionally an insertion of a nucleotide residue C. In further embodiments, the insertion is in the gene of PP_4950. In yet further embodiments, the insertion is at the 349th nucleotide (nt) residue of the 1320-nt-long coding sequence of PP_4950, optionally counted from the 5′ of the coding sequence.

In some embodiments, the at least one mutation(s) comprises, or consists essentially of, or yet further consists of a mutation at position 5679880, optionally an insertion of a nucleotide residue C. In further embodiments, the insertion is in the gene of PP_4983. In yet further embodiments, the insertion is at the 99th nucleotide (nt) residue of the 1860-nt-long coding sequence of PP_4983, optionally counted from the 5′ of the coding sequence.

In some embodiments, the at least one mutation(s) comprises, or consists essentially of, or yet further consists of a mutation at position 5749795, optionally the first nucleotide residue in the codon encoding T53 of the protein, from T optionally to C in the plus strand of the bacterial DNA (i.e., from A optionally to G in the protein coding strand), in the gene of PP_5043. In further embodiments, the mutated gene encodes a protein comprising a mutation of T53A.

In some embodiments, the at least one mutation(s) comprises, or consists essentially of, or yet further consists of a mutation at position 5773577, optionally the second nucleotide residue in the codon encoding A455 of the protein, from G optionally to A in the plus strand of the bacterial DNA (i.e., from C optionally to T in the protein coding strand), in the gene of betT-

In further embodiments, the mutated gene encodes a protein comprising a mutation of A455V.

In some embodiments, the at least one mutation(s) comprises, or consists essentially of, or yet further consists of a mutation at position 5895281 from A optionally to G in the intergenic region between the genes of PP_5167 and cysA, optionally at the intergenic position of (−34/+177).

In some embodiments, the at least one mutation(s) comprises, or consists essentially of, or yet further consists of a mutation at position 6032315, optionally a deletion in the intergenic region between the genes of PP_5729 and rpmG, further optionally at the intergenic position of (+22/+45).

In some embodiments, the engineered bacterium comprises an altered (i.e., increased or decreased) expression level of one or more of the genes disclosed herein, such as in Sheet 6 of Supplementary Data 3 compared to the wildtype grown on glucose or the bacterium without the at least one mutation(s) grown on glucose. In some embodiments, the engineered bacterium comprises an altered expression level of one or more of the genes disclosed in Sheet 7 of Supplementary Data 3 compared to the wildtype grown on glucose or the bacterium without the at least one mutation(s) grown on glucose. In some embodiments, the engineered bacterium comprises an altered expression level of one or more of the genes disclosed in Sheet 8 of Supplementary Data 3 compared to the wildtype grown on glucose or the bacterium without the at least one mutation(s) grown on glucose. In some embodiment, the engineered bacterium comprises an altered (increased or decreased) expression level as illustrated in FIG. 3D of the Appendices (filed in U.S. Provisional Patent Application Ser. Nos. 63/168,687, filed on Mar. 31, 2021) compared to the wildtype grown on glucose or the bacterium without the at least one mutation(s) grown on glucose. In some embodiments, the gene(s) having an altered expression in the engineered bacterium compared to the wildtype grown on glucose or the bacterium without the at least one mutation(s) grown on glucose comprises, or consists essentially of, or yet further consists of a gene selected from a cluster of orthologous group (COG) of an energy production and conversion gene, an amino acid metabolism and transport gene, a nucleotide metabolism and transport gene, a carbohydrate metabolism and transport gene, a coenzyme metabolism gene, a lipid metabolism gene, a translation gene, a transcription gene, a replication and repair gene, a cell wall/membrane/envelop biogenesis gene, a cell motility gene, a post-translational modification gene, an inorganic ion transport and metabolism gene, a secondary structure gene, a general functional prediction only gene, a signal transduction gene, an intracellular trafficking gene, or a defense mechanism gene. Definitions and gene lists for each COG are available at ncbi.nlm.nih.gov/research/cog/.

In some embodiments, the engineered bacterium comprises an increased expression level of one or more of genes selected from the group of: gnl (PP_1170), galP-I (PP_1173), PP_2585, fucD (PP_2831), PP_2834, PP_2835, PP_2836, PP_2837, ptxD (PP_3376), kguT (PP_3377), kguK (PP_3378), kguE (PP_3379), ptxS (PP_3380), or pyk (PP_4301), compared to the wildtype grown on glucose or the bacterium without the at least one mutation(s) grown on glucose.

In some embodiments, the engineered bacterium comprises a decreased expression level of one or more of genes selected from the group of: gapA (PP_1009), edd (PP_1010), glk (PP_1011), gtsA (PP_1015), gtsB (PP_1016), gtsC (PP_1017), gtsD (PP_1018), yeaD (PP_1020), zwfA (PP_1022), eda (PP_1024), pykA (PP_1362), gcd (PP_1444), oprB-II (PP_1445), eno (PP_1612), PP_3382, PP_3383, PP_3384, gnuK (PP_3416), gntT (PP_3417), oprB-III (PP_3570), or zwfB (PP_4042), compared to the wildtype grown on glucose or the bacterium without the at least one mutation(s) grown on glucose.

In some embodiments, the bacterium has a growth rate in a range from 0.20 h₋₁ to 0.26 h₋₁, or any range or rate of each therebetween, such as 0.22 h₋₁ to 0.25 h₋₁.

In some embodiments, the bacterium has a xylose uptake catalytic rate up to 1.2 g per g of dry cell weight (DCW) per hour.

In some embodiments, the bacterium has a biomass yield equal to or more than 0.20 g xylose per g of DCW.

In some embodiments, the bacterium further comprises one or both of the following in a chromosome of the bacterium: a nucleic acid sequence encoding the heterologous protein(s), or a regulatory sequence directing expression of the heterologous protein in the bacterium optionally a constitutive promoter and 5′-untranslated region.

In some embodiments, the engineered bacterium comprises a polynucleotide encoding the at least one heterologous protein(s). In further embodiments, a regulatory sequence endogenous (i.e., not heterologous) to the bacterium directs the expression of the at least one heterologous protein(s). In some embodiments, the engineered bacterium further comprises (for example, engineered to comprise) a regulatory sequence directing the expression of the at least one heterologous protein(s). In some embodiments, the engineered bacterium further comprises a regulatory sequence directing the expression of the at least one heterologous protein(s), and the regulatory sequence comprises, or consists essentially of, or further consists of one or more of: a promoter, such as a BBa_J23110 promoter comprising, consisting essentially of, or yet further consisting of TTTACGGCTAGCTCAGTCCTAGGTACAATGCTAGC (SEQ ID NO: 9); a 5′-untranslated region, for example comprising, consisting essentially of, or yet further consisting of AACAACAGCTTAGAAGGAGGTCAAT (SEQ ID NO: 10); an insulating sequence, for example comprising, consisting essentially of, or yet further consisting of AGGCTGTCTCGTCTCGTCTC (SEQ ID NO: 11) or GCTGGGAGTTCGTAGACGGA (SEQ ID NO: 12) or both; or a terminator, for example a BBa_B1002 terminator comprising, consisting essentially of, or yet further consisting of CGCAAAAAACCCCGCTTCGGCGGGGTTITTICGC (SEQ ID NO: 13). In further embodiments, the component(s) of the regulatory sequence and the polynucleotide encoding the at least one heterologous protein(s) are in one nucleotide molecule and in an order of, from the 5′ to the 3′, an insulating sequence (if present), a promoter (if present), a 5′-untranslated region (if present), a polynucleotide encoding the at least one heterologous protein(s), an insulating sequence (if present), and a terminator (if present). In one embodiment, the engineered bacterium comprises a xylD expression cassette comprising, consisting essentially of, or further consisting of SEQ ID NO:6.

In some embodiments, the gene mutation(s) is engineered into the bacterium by a plasmid. Additionally or alternatively, the gene mutation(s) is engineered into the bacterium by culturing a bacterium comprising the at least one heterologous protein(s) of the Weimberg pathway in a culture medium comprising xylose as the sole carbon source or one of the carbon sources.

In another aspect, provided is an engineered Pseudomonas bacterium comprising at least one heterologous protein(s) (i.e., a protein heterologous to a Pseudomonas bacterium, for example, a wild type Pseudomonas bacterium or a wild type Pseudomonas putida KT2440) of the Leloir pathway and at least one mutation(s) in a chromosome of the bacterium.

In some embodiments, the at least one heterologous protein(s) comprises, or essentially consists of, or yet further consists of one or more of proteins selected from the group of: UDP-glucose 4-epimerase, galactose-1-phosphate uridylyltransferase, galactokinase, or galactose-1-epimerase.

In some embodiments, the at least one heterologous protein(s) comprises, or essentially consists of, or yet further consists of one or more of Escherichia coli K-12 MG1655 proteins selected from the group of: UDP-glucose 4-epimerase, galactose-1-phosphate uridylyltransferase, galactokinase, or galactose-1-epimerase.

In some embodiments, the one or more of protein(s) is encoded by a heterologous galETKM operon.

In some embodiments, the bacterium is Pseudomonas putida KT2440.

In some embodiments, the bacterium catabolizes galactose.

In some embodiments, the at least one mutation(s) comprises, or consists essentially of, or yet further consists of a mutation in a gene(s) or intergenic region(s) selected from the group of: PP_0501; Ecoli_galK; Ecoli_galE, prfC; colS; PP_0949; rpoN; PP_1013; gtsA; gtsC; gtsB; gtsD; gtsABCD; galETKM; oprB-I; oprB-I, yeaD (oprB-I/yeaD); PP_1033, cumA; zapE; PP_1366; oprB-II; mutS; PP_1770; rffE; PP_1948; pepN; cmpX; folD-II; clpP; PP_2750; PP_2907; tnpT-I; gpD; PP_3935, PP_3938; uvrY; PP_4171; pvdL; fliO; flgH; PP_4592; PP_4684; dnaJ; or any other gene or intergenic region as disclosed herein. In some embodiments, the at least one mutation(s) comprises, or consists essentially of, or yet further consists of a mutation in a gene(s) or intergenic region(s) selected from the group of: gtsA; gtsC; oprB-I; oprB-I, yeaD; or oprB-II. In some embodiments, the at least one mutation(s) comprises, or consists essentially of, or yet further consists of a mutation disclosed in Sheet 4 of Supplementary Data 3. In some embodiments, the at least one mutation(s) comprises, or essentially consists of, or yet further consists of one or more of gain-of-function mutation(s) in genes selected from the group of: gtsA; gtsC; gtsB; gtsD; gtsABCD; or another gene, a gene product of which transports galactose into the cytosol. In some embodiments, the at least one mutation(s) comprises, or essentially consists of, or yet further consists of one or more of loss-of-function mutation(s) in oprB-I/yeaD or oprB-II or both.

In some embodiments, a mutated gene comprising the at least one mutation(s) is PP_0165. In further embodiments, the mutated gene encodes a mutated protein of GGDEF domain-containing protein. In yet further embodiments, the mutated protein comprises a R316C mutation, for example caused by a nucleotide codon mutation from CGC to TGC. In some embodiments, a mutated gene comprising the at least one mutation(s) is PP_0180. In further embodiments, the mutated gene encodes a protein of cytochrome c family protein tRNA N6-adenosine(37)-threonylcarbamoyltransferase. In some embodiments, the nucleotide codon encoding G548 of the protein is mutated from GGC to GGT. In some embodiments, a mutated gene comprising the at least one mutation(s) is tsaD. In further embodiments, the mutated gene encodes a mutated protein of complex transferase subunit tsaD. In some embodiments, the mutated protein comprises a Q141L mutation, for example caused by a nucleotide codon mutation from CAG to CTG. In some embodiments, a mutated gene comprising the at least one mutation(s) is relA. In further embodiments, the mutated gene encodes a mutated protein of ATP:GTP 3′-pyrophosphotransferase. In some embodiments, the mutated protein comprises a D203G mutation, for example caused by a nucleotide codon mutation from GAT to GGT. In some embodiments, a mutated intergenic region comprising the at least one mutation(s) is in the chromosome between the mvaB gene encoding hydroxymethylglutaryl-CoA lyase and the PP_mr44 gene encoding CrcZ ncRNA. In some embodiment, in addition to one or more mutation as disclosed in this paragraph, the engineered bacterium comprises a further mutation as disclosed herein.

In some embodiments, the at least one mutation(s) comprises, or consists essentially of, or yet further consists of a mutation at position 591535, optionally the second nucleotide residue in the codon encoding V260 of the protein, from T optionally to A in the gene of PP_0501. In further embodiments, the mutated gene encodes a protein comprising a mutation of V260D.

In some embodiments, the at least one mutation(s) comprises, or consists essentially of, or yet further consists of a mutation at position 1013189, optionally the second nucleotide residue in the codon encoding C170 of the protein, from C optionally to G in the plus strand of the bacterial DNA (i.e., from G optionally to C in the protein coding strand) in the gene of Ecoli_galK. In further embodiments, the mutated gene encodes a protein comprising a mutation of C170S.

In some embodiments, the at least one mutation(s) comprises, or consists essentially of, or yet further consists of a mutation at position 1015820 from A optionally to C in the intergenic region between the genes of Ecoli_galE and prfC, optional at the intergenic position of (−47/−721).

In some embodiments, the at least one mutation(s) comprises, or consists essentially of, or yet further consists of a mutation at position 1047840, optionally the second nucleotide residue in the codon encoding S233of the protein, from C optionally to T in the gene of colS. In further embodiments, the mutated gene encodes a protein comprising a mutation of S233L.

In some embodiments, the at least one mutation(s) comprises, or consists essentially of, or yet further consists of a mutation at position 1098137, optionally the first nucleotide residue in the codon encoding G173 of the protein, from C optionally to A in the plus strand of the bacterial DNA (i.e., from G optionally to T in the protein coding strand) in the gene of PP_0949. In further embodiments, the mutated gene encodes a protein comprising a mutation of G173W.

In some embodiments, the at least one mutation(s) comprises, or consists essentially of, or yet further consists of a mutation at position 1099817, optionally the first nucleotide residue in the codon encoding T400 of the protein, from T optionally to G in the plus strand of the bacterial DNA (i.e., from A optionally to C in the protein coding strand) in the gene of rpoN. In further embodiments, the mutated gene encodes a protein comprising a mutation of T400P.

In some embodiments, the at least one mutation(s) comprises, or consists essentially of, or yet further consists of a mutation at position 1160716, optionally the first nucleotide residue in the codon encoding L307 of the protein, from C optionally to G in the gene of PP_1013. In further embodiments, the mutated gene encodes a protein comprising a mutation of L307V.

In some embodiments, the at least one mutation(s) comprises, or consists essentially of, or yet further consists of a mutation at position 1163121, optionally the second nucleotide residue in the codon encoding A100 of the protein, from C optionally to T in the gene of gtsA. In further embodiments, the mutated gene encodes a protein comprising a mutation of A100V.

In some embodiments, the at least one mutation(s) comprises, or consists essentially of, or yet further consists of a mutation at position 1163732, optionally the first nucleotide residue in the codon encoding N304 of the protein, from A optionally to G in the gene of gtsA. In further embodiments, the mutated gene encodes a protein comprising a mutation of N304D.

In some embodiments, the at least one mutation(s) comprises, or consists essentially of, or yet further consists of a mutation at position 1163733, optionally the second nucleotide residue in the codon encoding N304 of the protein, from A optionally to G in the gene of gtsA. In further embodiments, the mutated gene encodes a protein comprising a mutation of N304S.

In some embodiments, the at least one mutation(s) comprises, or consists essentially of, or yet further consists of a mutation at position 1164101, optionally the first nucleotide residue in the codon encoding A427 of the protein, from G optionally to A in the gene of gtsA. In further embodiments, the mutated gene encodes a protein comprising a mutation of A427T.

In some embodiments, the at least one mutation(s) comprises, or consists essentially of, or yet further consists of a mutation at position 1165507, optionally the first nucleotide residue in the codon encoding F122 of the protein, from T optionally to C in the gene of gtsC. In further embodiments, the mutated gene encodes a protein comprising a mutation of F122L.

In some embodiments, the at least one mutation(s) comprises, or consists essentially of, or yet further consists of a mutation at position 1165540, optionally the first nucleotide residue in the codon encoding L133 of the protein, from C optionally to T in the gene of gtsC. In further embodiments, the mutated gene encodes a protein comprising a mutation of L133F.

In some embodiments, the at least one mutation(s) comprises, or consists essentially of, or yet further consists of a mutation at position 1165856, optionally the second nucleotide residue in the codon encoding T238 of the protein, from C optionally to T in the gene of gtsC. In further embodiments, the mutated gene encodes a protein comprising a mutation of T238I.

In some embodiments, the at least one mutation(s) comprises, or consists essentially of, or yet further consists of a mutation at position 1167740, optionally the first nucleotide residue in the codon encoding Q185 of the protein, from C optionally to T in the gene of oprB-I. In further embodiments, the mutated gene encodes a truncated protein terminated at Q185, i.e., only consisting of the first 184 amino acid residues.

In some embodiments, the at least one mutation(s) comprises, or consists essentially of, or yet further consists of a mutation at position 1167912, optionally an insertion of a nucleotide residue A. In further embodiments, the insertion is in the gene of oprB-I. In yet further embodiments, the insertion is at the 725th nucleotide (nt) residue of the 1344-nt-long coding sequence of oprB-I, optionally counted from the 5′ of the coding sequence.

In some embodiments, the at least one mutation(s) comprises, or consists essentially of, or yet further consists of a mutation at position 1168578 from C optionally to A in the intergenic region between the genes of oprB-I and yeaD, optionally at the intergenic position of (+47/−71).

In some embodiments, the at least one mutation(s) comprises, or consists essentially of, or yet further consists of a mutation at position 1168596 from G optionally to T in the intergenic region between the genes of oprB-I and yeaD, optionally at the intergenic position of (+65/−53).

In some embodiments, the at least one mutation(s) comprises, or consists essentially of, or yet further consists of a mutation at position 1185783, optionally an insertion of a nucleotide residue G. In further embodiments, the insertion is in the intergenic region between the genes of PP_1033 and CumA, optionally at the intergenic position of (−51/−120).

In some embodiments, the at least one mutation(s) comprises, or consists essentially of, or yet further consists of a mutation at position 1505141, optionally a deletion in the gene of zapE. In further embodiments the deletion comprises, or consists essentially of, or further consists of a deletion at the 597th nt residue of the 1095-nt-long coding sequence of zapE, optionally counted from the 5′ of the coding sequence.

In some embodiments, the at least one mutation(s) comprises, or consists essentially of, or yet further consists of a mutation at position 1560509, optionally the second nucleotide residue in the codon encoding G99 of the protein, from C optionally to T in the plus strand of the bacterial DNA (i.e., from G optionally to A in the protein coding strand) in the gene of PP_1366. In further embodiments, the mutated gene encodes a protein comprising a mutation of G99D.

In some embodiments, the at least one mutation(s) comprises, or consists essentially of, or yet further consists of a mutation at position 1652918, optionally the third nucleotide residue in the codon encoding Y265 of the protein, from G optionally to A in the plus strand of the bacterial DNA (i.e., from C optionally to T in the protein coding strand) in the gene of oprB-II.

In some embodiments, the at least one mutation(s) comprises, or consists essentially of, or yet further consists of a mutation at position 1828233, optionally the second nucleotide residue in the codon encoding V671 of the protein, from A optionally to C in the plus strand of the bacterial DNA (i.e., from T optionally to G in the protein coding strand) in the gene of mutS. In further embodiments, the mutated gene encodes a protein comprising a mutation of V671G.

In some embodiments, the at least one mutation(s) comprises, or consists essentially of, or yet further consists of a mutation at position 1982697, optionally the first nucleotide residue in the codon encoding P457 of the protein, from C optionally to T in the gene of PP_1770. In further embodiments, the mutated gene encodes a protein comprising a mutation of P457S.

In some embodiments, the at least one mutation(s) comprises, or consists essentially of, or yet further consists of a mutation at position 2043352, optionally an insertion of a nucleotide residue G. In further embodiments, the insertion is in the gene of rffE. In yet further embodiments, the insertion is at the 1120th nucleotide (nt) residue of the 1143-nt-long coding sequence of rffE, optionally counted from the 5′ of the coding sequence.

In some embodiments, the at least one mutation(s) comprises, or consists essentially of, or yet further consists of a mutation at position 2209475, optionally the second nucleotide residue in the codon encoding N387 of the protein, from A optionally to T in the gene of PP_1948. In further embodiments, the mutated gene encodes a protein comprising a mutation of N387I.

In some embodiments, the at least one mutation(s) comprises, or consists essentially of, or yet further consists of a mutation at position 2293757, optionally the first nucleotide residue in the codon encoding D350 of the protein, from G optionally to A in the gene of pepN. In further embodiments, the mutated gene encodes a protein comprising a mutation of D350N.

In some embodiments, the at least one mutation(s) comprises, or consists essentially of, or yet further consists of a mutation at position 2384391, optionally the first nucleotide residue in the codon encoding Q227 of the protein, from C optionally to A in the gene of cmpX. In further embodiments, the mutated gene encodes a protein comprising a mutation of Q227K.

In some embodiments, the at least one mutation(s) comprises, or consists essentially of, or yet further consists of a mutation at position 2590436, optionally the third nucleotide residue in the codon encoding Q17 of the protein, from C optionally to G in the plus strand of the bacterial DNA (i.e., from G optionally to C in the protein coding strand) in the gene of folD-II. In further embodiments, the mutated gene encodes a protein comprising a mutation of Q17H.

In some embodiments, the at least one mutation(s) comprises, or consists essentially of, or yet further consists of a mutation at position 2633849, optionally the third nucleotide residue in the codon encoding H173 of the protein, from C optionally to T in the gene of clpP.

In some embodiments, the at least one mutation(s) comprises, or consists essentially of, or yet further consists of a mutation at position 3138381, optionally the third nucleotide residue in the codon encoding A195 of the protein, from G optionally to A in the gene of PP_2750.

In some embodiments, the at least one mutation(s) comprises, or consists essentially of, or yet further consists of a mutation at position 3311259, optionally the first nucleotide residue in the codon encoding G188 of the protein, from C optionally to G in the plus strand of the bacterial DNA (i.e., from G optionally to C in the protein coding strand) in the gene of PP_2907. In further embodiments, the mutated gene encodes a protein comprising a mutation of G188R.

In some embodiments, the at least one mutation(s) comprises, or consists essentially of, or yet further consists of a mutation at position 3385634, optionally the second nucleotide residue in the codon encoding A105 of the protein, from C optionally to T in the gene of tnpT-l. In further embodiments, the mutated gene encodes a protein comprising a mutation of A105V.

In some embodiments, the at least one mutation(s) comprises, or consists essentially of, or yet further consists of a mutation at position 3450733, optionally the third nucleotide residue in the codon encoding R45 of the protein, from C optionally to A in the gene of gpD.

In some embodiments, the at least one mutation(s) comprises, or consists essentially of, or yet further consists of a mutation at position 4445476, from T optionally to C in the intergenic region between the genes of PP_3935 and PP_3938, optionally at the intergenic position of (+141/−398).

In some embodiments, the at least one mutation(s) comprises, or consists essentially of, or yet further consists of a mutation at position 4640692, optionally the second nucleotide residue in the codon encoding M20 of the protein, from A optionally to T in the plus strand of the bacterial DNA (i.e., from T optionally to A in the protein coding strand) in the gene of uvrY. In further embodiments, the mutated gene encodes a protein comprising a mutation of M20K.

In some embodiments, the at least one mutation(s) comprises, or consists essentially of, or yet further consists of a mutation at position 4717074, optionally the second nucleotide residue in the codon encoding R104 of the protein, from G optionally to A in the gene of PP_4171. In further embodiments, the mutated gene encodes a protein comprising a mutation of R104H.

In some embodiments, the at least one mutation(s) comprises, or consists essentially of, or yet further consists of a mutation at position 4829283, optionally the first nucleotide residue in the codon encoding E2301 of the protein, from C optionally to G in the plus strand of the bacterial DNA (i.e., from G optionally to C in the protein coding strand) in the gene of pvdL. In further embodiments, the mutated gene encodes a protein comprising a mutation of E2301Q.

In some embodiments, the at least one mutation(s) comprises, or consists essentially of, or yet further consists of a mutation at position 4953768, optionally a deletion in the gene of fliO. In further embodiments, the deletion comprises, or consists essentially of, or further consists of a deletion at the 74th nt residue of the 435-nt-long coding sequence of fliO, optionally counted from the 5′ of the coding sequence.

In some embodiments, the at least one mutation(s) comprises, or consists essentially of, or yet further consists of a mutation at position 4983564, optionally a deletion of nt residue of C in the gene of flgH. In further embodiments, the deletion comprises, or consists essentially of, or further consists of a deletion the 30th nt residue of the 696-nt-long coding sequence of flgH, optionally counted from the 5′ of the coding sequence.

In some embodiments, the at least one mutation(s) comprises, or consists essentially of, or yet further consists of a mutation at position 5216752, optionally the third nucleotide residue in the codon encoding A65 of the protein, from G optionally to A in the plus strand of the bacterial DNA (i.e., from C optionally to T in the protein coding strand) in the gene of PP_4592.

In some embodiments, the at least one mutation(s) comprises, or consists essentially of, or yet further consists of a mutation at position 5328681, optionally the third nucleotide residue in the codon encoding M106 of the protein, from G optionally to C in the gene of PP_4684. In further embodiments, the mutated gene encodes a protein comprising a mutation of M1061.

In some embodiments, the at least one mutation(s) comprises, or consists essentially of, or yet further consists of a mutation at position 5379892, optionally the third nucleotide residue in the codon encoding V182 of the protein, from C optionally to T in the plus strand of the bacterial DNA (i.e., from G optionally to A in the protein coding strand) in the gene of dnaJ.

In some embodiments, the engineered bacterium comprises an altered (i.e., increased or decreased) expression level of one or more of the genes disclosed herein, such as in Sheet 6 of Supplementary Data 3 compared to the wildtype grown on glucose or the bacterium without the at least one mutation(s) grown on glucose. In some embodiments, the engineered bacterium comprises an altered expression level of one or more of the genes disclosed in Sheet 7 of Supplementary Data 3 compared to the wildtype grown on glucose or the bacterium without the at least one mutation(s) grown on glucose. In some embodiments, the engineered bacterium comprises an altered expression level of one or more of the genes disclosed in Sheet 8 of Supplementary Data 3 compared to the wildtype grown on glucose or the bacterium without the at least one mutation(s) grown on glucose. In some embodiment, the engineered bacterium comprises an altered (increased or decreased) expression level as illustrated in FIG. 3D of the Appendices (filed in U.S. Provisional Patent Application Ser. Nos. 63/168,687, filed on Mar. 31, 2021) compared to the wildtype grown on glucose or the bacterium without the at least one mutation(s) grown on glucose. In some embodiments, the gene(s) having an altered expression in the engineered bacterium compared to the wildtype grown on glucose or the bacterium without the at least one mutation(s) grown on glucose comprises, or consists essentially of, or yet further consists of a gene selected from a cluster of orthologous group (COG) of an energy production and conversion gene, a cell cycle control and mitosis gene, an amino acid metabolism and transport gene, a nucleotide metabolism and transport gene, a carbohydrate metabolism and transport gene, a coenzyme metabolism gene, a lipid metabolism gene, a translation gene, a transcription gene, a replication and repair gene, a cell wall/membrane/envelop biogenesis gene, a cell motility gene, a post-translational modification gene, an inorganic ion transport and metabolism gene, a secondary structure gene, a general functional prediction only gene, a signal transduction gene, a intracellular trafficking gene, or a defense mechanism gene. Definitions and gene lists for each COG are available at ncbi.nlm.nih.gov/research/cog/.

In some embodiments, the engineered bacterium comprises an increased expression level of one or more of genes selected from the group of: gtsABCD; gapA (PP_1009); edd (PP_1010); glk (PP_1011); gtsA (PP_1015); gtsB (PP_1016); gtsC (PP_1017); gtsD (PP_1018); oprB-I (PP_1019); gnl (PP_1170); galP-I (PP_1173); pykA (PP_1362); PP_2585; fucD (PP_2831); PP_2834; PP_2835; PP_2836; PP_2837; or pyk (PP_4301), compared to the wildtype grown on glucose or the bacterium without the at least one mutation(s) grown on glucose. In some embodiments, the engineered bacterium comprises a decreased or increased expression level of oprB-II or gdc or both compared to the wildtype grown on glucose or the bacterium without the at least one mutation(s) grown on glucose. In some embodiments, the engineered bacterium comprises a decreased expression level of one or more of the following genes compared to the wildtype grown on glucose or the bacterium without the at least one mutation(s) grown on glucose: ptxD (PP_3376); kguT (PP_3377); kguK (PP_3378); kguE (PP_3379); ptxS (PP_3380); PP_3382; PP_3383; PP_3384; gnuK (PP_3416); gntT (PP_3417); oprB-III (PP_3570); or zwfB (PP_4042).

In some embodiments, the bacterium has a growth rate in a range from 0.33 h and 0.52 h₋₁, or any range or rate of each therebetween.

In some embodiments, the bacterium further comprises one or both of the following in a chromosome of the bacterium: a nucleic acid sequence encoding the heterologous protein(s), or a regulatory sequence directing expression of the heterologous protein in the bacterium optionally a constitutive promoter and 5′-untranslated region.

In some embodiments, the engineered bacterium comprises a polynucleotide encoding the at least one heterologous protein(s). In further embodiments, a regulatory sequence endogenous (i.e., not heterologous) to the bacterium directs the expression of the at least one heterologous protein(s). In some embodiments, the engineered bacterium further comprises (for example, engineered to comprise) a regulatory sequence directing the expression of the at least one heterologous protein(s). In some embodiments, the engineered bacterium further comprises a regulatory sequence directing the expression of the at least one heterologous protein(s), and the regulatory sequence comprises, or consists essentially of, or further consists of one or more of: a promoter, such as a BBa_J23110 promoter comprising, consisting essentially of, or yet further consisting of tttacggetagctcagtectaggtacaatgctagc; a 5′-untranslated region, for example comprising, consisting essentially of, or yet further consisting of aacaacagcttagaaggaggtcaat; an insulating sequence, for example comprising, consisting essentially of, or yet further consisting of AGGCTGTCTCGTCTCGTCTC (SEQ ID NO:11) or GCTGGGAGTTCGTAGACGGA (SEQ ID NO:12) or both; or a terminator, for example a BBa_B1002 terminator comprising, consisting essentially of, or yet further consisting of CGCAAAAAACCCCGCTTCGGCGGGGTTTTCGC (SEQ ID NO:13). In further embodiments, the component(s) of the regulatory sequence and the polynucleotide encoding the at least one heterologous protein(s) are in one nucleotide molecule and in an order of, from the 5′ to the 3′, an insulating sequence (if present), a promoter (if present), a 5′-untranslated region (if present), a polynucleotide encoding the at least one heterologous protein(s), an insulating sequence (if present), and a terminator (if present).

In some embodiments, the gene mutation(s) is engineered into the bacterium by a plasmid. Additionally or alternatively, the gene mutation(s) is engineered into the bacterium by culturing a bacterium comprising the at least one heterologous protein(s) of the Leloir pathway in a culture medium comprising galactose as the sole carbon source or one of the carbon sources.

In some embodiments, the engineered bacterium further comprises a heterologous gene expressing a gene product. In further embodiments, the gene product is one or more enzymes producing indigoidine.

In yet another aspect, provided is a plurality of the engineered bacterium as disclosed herein. In some embodiments, the bacteria in the plurality are the same or different from each other.

In a further aspect, provided is a composition comprising, or essentially consisting of, or yet further consisting of the plurality of bacterium as disclosed herein, and a carrier.

In one aspect, provided is a method for producing a metabolite. The method comprises, or essentially consists of, or yet further consists of culturing the engineered bacterium as disclosed herein or a plurality thereof in a culture medium. In some embodiments, the method further comprises isolating the metabolite from the culture medium or the bacterium or both.

In a further aspect, provided is a method for producing a metabolite. The method comprises, or essentially consists of, or yet further consists of culturing the engineered bacterium as disclosed herein or a plurality thereof in a culture medium. In some embodiments, the method further comprises isolating the metabolite from the culture medium or the bacterium or both. In some embodiments, the engineered bacterium further comprises a heterologous gene expressing a gene product. In further embodiments, the gene product is the produced metabolite. In some embodiments, the heterologous gene expresses an enzyme catalyzing which in turn producing the metabolite. One exemplified method is detailed in the Appendices (filed in U.S. Provisional Patent Application Ser. Nos. 63/168,687, filed on Mar. 31, 2021) for producing indigoidine.

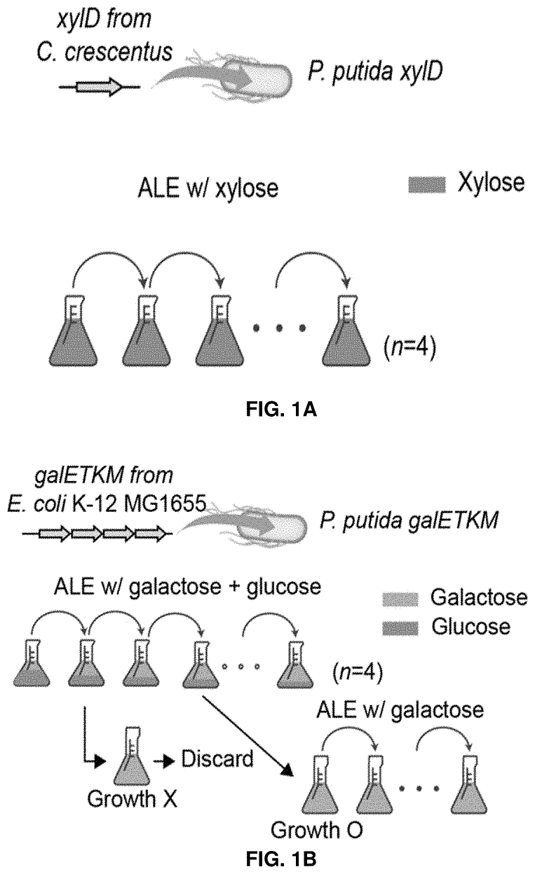

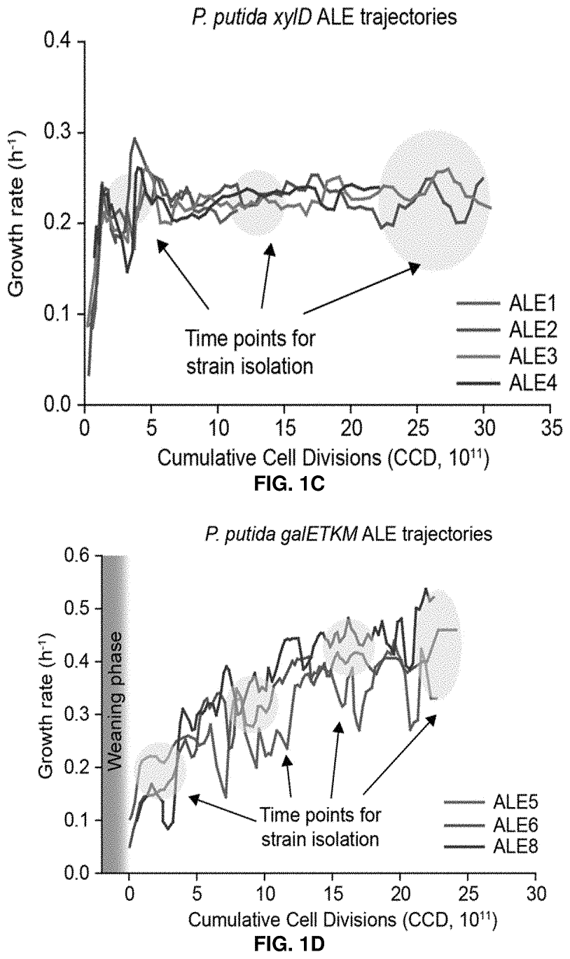

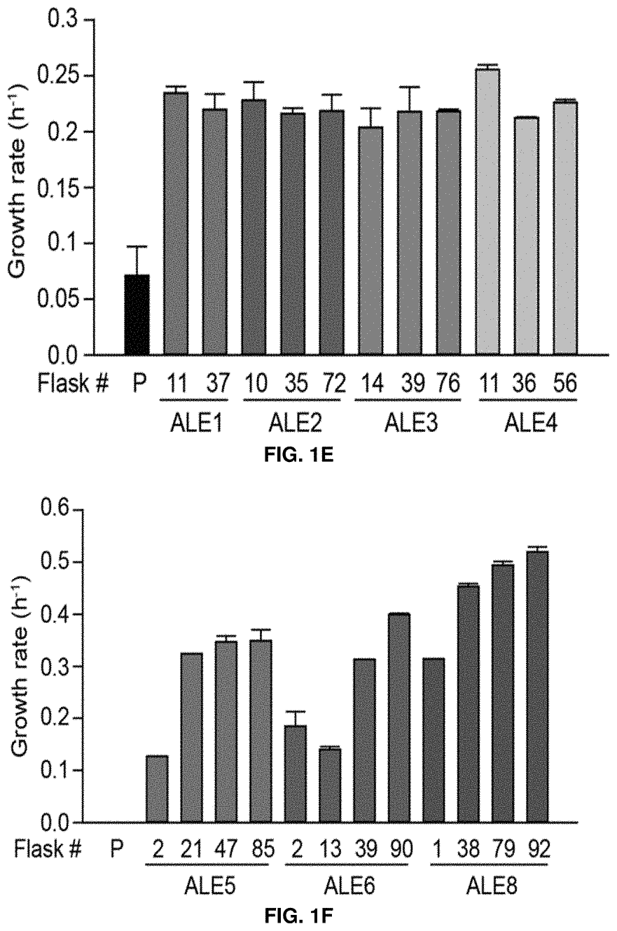

In another aspect, provided is a method for generating an engineered bacterium. The method comprises, or essentially consists of, or yet further consists of culturing a bacterium that comprises at least one heterologous protein(s), wherein the at least one heterologous protein(s) catabolizes a non-native carbon source in a culture medium, and wherein the culture medium comprises the non-native carbon source as the sole carbon source; monitoring growth rate of the cultured bacterium; and isolating a progeny of the cultured bacterium when or prior to the growth rate reaching a Plateau. One exemplified method is detailed in the Appendices for producing a P. putida catabolizing xylose. See, e.g., the illustration in FIG. 1A of the Appendices (filed in U.S. Provisional Patent Application Ser. Nos. 63/168,687, filed on Mar. 31, 2021).

In yet another aspect, provided is a method for generating an engineered bacterium. The method comprises, or essentially consists of, or yet further consists of culturing a bacterium that comprises at least one heterologous protein(s) in a series of culture medium, wherein the at least one heterologous protein(s) catabolizes a non-native carbon source, wherein the series of culture medium comprises a gradually increased ratio of the non-native carbon source over a native carbon source; monitoring growth rate of the cultured bacterium; and isolating a progeny of the cultured bacterium when or prior to the growth rate reaching a Plateau. One exemplified method is detailed in the Appendices for producing a P. putida catabolizing galactose. See, e.g., the illustration in FIG. 1B of the Appendices (filed in U.S. Provisional Patent Application Ser. Nos. 63/168,687, filed on Mar. 31, 2021).

Also provided is an engineered bacterium generated by the method as disclosed herein, an engineered bacterium as disclosed herein, an engineered bacterium for use in biomass processing processes, and a kit for use in the method as disclosed herein. In some embodiments, the kit comprises, or essentially consists of, or yet further consists of a bacterium as disclosed herein or a plurality thereof, and instructions. In some embodiments, the kit comprises, or essentially consists of, or yet further consists of the bacterium that comprises at least one heterologous protein(s), the non-native carbon, and instructions.

In one aspect, provided is an engineered or isolated polynucleotide comprising, or consisting essentially of, or yet further consisting of a mutated gene as disclosed herein, for example in Supplementary Data 3. Further provided is a vector, such as a viral vector or a non-viral vector, comprising, or consisting essentially of, or yet further consisting of the polynucleotide. In some embodiments, the vector further comprises a regulatory sequence directing the expression of the mutated gene. In further aspect, provided is a polynucleotide or a vector suitable for introducing a mutation as disclosed herein into a cell, such as a bacterium.

One non-limiting example is a clustered regularly interspaced short palindromic repeats (CRISPR) system comprising, or consisting essentially of, or yet further consisting of a CRISPR-associated endonuclease (such as Cas9 or Cas 12a), a guide polynucleotide (such as a guide RNA) directing the CRISPR-associated protein to the mutation site, and optionally a template polynucleotide comprising the mutation and homologous sequences immediately upstream and downstream of the mutation.

In another aspect, provided is an engineered or isolated polypeptide comprising, or consisting essentially of, or yet further consisting of a mutated protein or a fragment thereof comprising a mutation as disclosed herein, for example in Supplementary Data 3 (filed in U.S. Provisional Patent Application Ser. Nos. 63/168,687, filed on Mar. 31, 2021).

In some embodiments, the engineered bacterium as disclosed herein does not comprise those disclosed in Table S1 of the Appendices (filed in U.S. Provisional Patent Application Ser. Nos. 63/168,687, filed on Mar. 31, 2021) except the one identified in the last line. For example, the engineered bacterium as disclosed herein does not comprise one or more of the following: a P. putida S12 with heterologous expression of xylAB from Escherichia coli (i.e., E. coli), a P. putida KT2440 with heterologous expression of xylAB from E. coli, a P. putida EM42 (KT2440 derivative) with heterologous expression of xylAB from E. coli, a P. putida KT2440 with heterologous expression of xylAB from E. coli, a P. putida KT2440 with heterologous expression of xylAB, tal, tkt, xylE from E. coli, a P. putida S12 with heterologous expression of xylXABCD from Caulobacter crescentus, a P. putida KT2440 with heterologous expression of PVLB18550, PVLB18555, PVLB18560, PVLB18565 from P. taiwanensis VLB120, a P. putida KT2440 with heterologous expression of xylD from Caulobacter crescentus, a P. putida KT2440 with heterologous expression of PVLB18555 and PVLB18565 from P. taiwanensis VLB120, a P. putida KT2440 with heterologous expression of araAB from Burkholderia ambifaria, dgoKAD from P. fluorescens SBW25, araAB and galP from E. coli, or P. putida KT2440 with heterologous expression of galETKM from E. coli.

In some embodiments, the bacterium as disclosed herein is engineered from a Pseudomonas bacterium, optionally a Pseudomonas putida (i.e., P. putida) bacterium, such as P. putida S12, P, putida KT2440, P, putida EM42, ATCC 12633, CCUG 12690, CFBP 2066, CIP 52.191, DSM 291, DSM 7314, HAMBI 7, IFO 14164, JCM 13063, JCM 20120, LMG 2257, NBRC 14164, NCAIM B.01634, NCCB 68020, NCCB 72006, NCTC 10936, or another Pseudomonas putida strain. In further embodiments, the bacterium as disclosed herein is engineered from a wildtype Pseudomonas bacterium, optionally a wildtype Pseudomonas putida. In further embodiments, the bacterium as disclosed herein is engineered from a Pseudomonas bacterium, optionally a Pseudomonas putida, which may have engineered, for example, to comprise a gene heterologous to the wildtype or to lack a gene endogenous to the wildtype.

### REFERENCES CITED HEREIN

Antonovsky, N., Gleizer, S., Noor, E., Zohar, Y., Herz, E., Barenholz, U., Zelebuch, L., Amram, S., Wides, A., Tepper, N., Davidi, D., Bar-On, Y., Bareia, T., Wernick, D. G., Shani, I., Malitsky, S., Jona, G., Bar-Even, A., Milo, R., 2016. Sugar Synthesis from C02 in Escherichia coli. Cell 166, 115-125. doi:10.1016/j.cell.2016.05.064
Bailey, S. F., Hinz, A., Kassen, R., 2014. Adaptive synonymous mutations in an experimentally evolved Pseudomonas fluorescens population. Nat. Commun. 5, 4076. doi:10.1038/ncomms5076
Banerjee, D., Eng, T., Lau, A. K., Sasaki, Y., Wang, B., Chen, Y., Prahl, J.-P., Singan, V. R., Herbert, R. A., Liu, Y., Tanjore, D., Petzold, C. J., Keasling, J. D., Mukhopadhyay, A., 2020. Genome-scale metabolic rewiring improves titers rates and yields of the non-native product indigoidine at scale. Nat. Commun. 11, 5385. doi:10.1038/s41467-020-19171-4
Bator, I., Wittgens, A., Rosenau, F., Tiso, T., Blank, L. M., 2019. Comparison of Three Xylose Pathways in Pseudomonas putida KT2440 for the Synthesis of Valuable Products. Front. Bioeng. Biotechnol. 7,480. doi:10.3389/fbioe.2019.00480
Belda, E., van Heck, R. G. A., José Lopez-Sanchez, M., Cruveiller, S., Barbe, V., Fraser, C., Klenk, H.-P., Petersen, J., Morgat, A., Nikel, P. I., Vallenet, D., Rouy, Z., Sekowska, A., Martins Dos Santos, V. A. P., de Lorenzo, V., Danchin, A., Médigue, C., 2016. The revisited genome of Pseudomonas putida KT2440 enlightens its value as a robust metabolic chassis. Environ. Microbiol. 18, 3403-3424. doi:10.1111/1462-2920.13230
Bentley, G. J., Narayanan, N., Jha, R. K., Salvachúa, D., Elmore, J. R., Peabody, G. L., Black, B. A., Ramirez, K., De Capite, A., Michener, W. E., Werner, A. Z., Klingeman, D. M., Schindel, H. S., Nelson, R., Foust, L., Guss, A. M., Dale, T., Johnson, C. W., Beckham, G. T., 2020. Engineering glucose metabolism for enhanced muconic acid production in Pseudomonas putida KT2440. Metab. Eng. 59, 64-75. doi:10.1016/j.ymben.2020.01.001
Buckel, P., Zehelein, E., 1981. Expression of Pseudomonas fluorescens D-galactose dehydrogenase in E. coli. Gene 16, 149-159. doi:10.1016/0378-1119(81)90071-8
Deatherage, D. E., Barrick, J. E., 2014. Identification of mutations in laboratory-evolved microbes from next-generation sequencing data using breseq. Methods Mol. Biol. 1151, 165-188. doi:10.1007/978-1-4939-0554-6_12
Del Castillo, T., Duque, E., Ramos, J. L., 2008. A set of activators and repressors control peripheral glucose pathways in Pseudomonas putida to yield a common central intermediate. J. Bacteriol. 190, 2331-2339. doi:10.1128/JB.01726-07
Dokter, P., Pronk, J. T., Schie, B. J., Dijken, J. P., Duine, J. A., 1987. The in vivo and in vitro substrate specificity of quinoprotein glucose dehydrogenase of Acinetobacter calcoaceticus LMD79.41. FEMS Microbiol. Lett. 43, 195-200. doi:10.1111/j.1574-6968.1987.tb02122.x
Dvořák, P., de Lorenzo, V., 2018. Refactoring the upper sugar metabolism of Pseudomonas putida for co-utilization of cellobiose, xylose, and glucose. Metab. Eng. 48, 94-108. doi:10.1016/j.ymben.2018.05.019
Elmore, J. R., Dexter, G. N., Salvachúa, D., O'Brien, M., Klingeman, D. M., Gorday, K., Michener, J. K., Peterson, D. J., Beckham, G. T., Guss, A. M., 2020. Engineered Pseudomonas putida simultaneously catabolizes five major components of corn stover lignocellulose: Glucose, xylose, arabinose, p-coumaric acid, and acetic acid. Metab. Eng. 62, 62-71. doi:10.1016/j.ymben.2020.08.001
Enquist-Newman, M., Faust, A. M. E., Bravo, D. D., Santos, C. N. S., Raisner, R. M., Hanel, A., Sarvabhowman, P., Le, C., Regitsky, D. D., Cooper, S. R., Peereboom, L., Clark, A., Martinez, Y., Goldsmith, J., Cho, M. Y., Donohoue, P. D., Luo, L., Lamberson, B., Tamrakar, P., Kim, E. J., Villari, J. L., Gill, A., Tripathi, S. A., Karamchedu, P., Paredes, C. J., Rajgarhia, V., Kotlar, H. K., Bailey, R. B., Miller, D. J., Ohler, N. L., Swimmer, C., Yoshikuni, Y., 2014. Efficient ethanol production from brown macroalgae sugars by a synthetic yeast platform. Nature 505, 239-243. doi:10.1038/nature12771
Guzmán, G. I., Sandberg, T. E., LaCroix, R. A., Nyerges, A., Papp, H., de Raad, M., King, Z. A., Hefner, Y., Northen, T. R., Notebaart, R. A., PdJ, C., Palsson, B. O., Papp, B., Feist, A. M., 2019. Enzyme promiscuity shapes adaptation to novel growth substrates. Mol. Syst. Biol. 15, e8462. doi:10.15252/msb.20188462
Isikgor, F. H., Becer, C. R., 2015. Lignocellulosic biomass: a sustainable platform for the production of bio-based chemicals and polymers. Polym. Chem. 6, 4497-4559. doi:10.1039/C5PY00263J
Kang, C. W., Lim, H. G., Yang, J., Noh, M. H., Seo, S. W., Jung, G. Y., 2018. Synthetic auxotrophs for stable and tunable maintenance of plasmid copy number. Metab. Eng. 48, 121-128. doi:10.1016/j.ymben.2018.05.020
Köhler, K. A. K., Blank, L. M., Frick, O., Schmid, A., 2015. D-Xylose assimilation via the Weimberg pathway by solvent-tolerant Pseudomonas taiwanensis VLB120. Environ. Microbiol. 17, 156-170. doi:10.1111/1462-2920.12537
LaCroix, R. A., Palsson, B. O., Feist, A. M., 2017. A model for designing adaptive laboratory evolution experiments. Appl. Environ. Microbiol. 83. doi:10.1128/AEM.03115-16
LaCroix, R. A., Sandberg, T. E., O'Brien, E. J., Utrilla, J., Ebrahim, A., Guzman, G. I., Szubin, R., Palsson, B. O., Feist, A. M., 2015. Use of adaptive laboratory evolution to discover key mutations enabling rapid growth of Escherichia coli K-12 MG1655 on glucose minimal medium. Appl. Environ. Microbiol. 81, 17-30. doi:10.1128/AEM.02246-14
Langmead, B., Salzberg, S. L., 2012. Fast gapped-read alignment with Bowtie 2. Nat. Methods 9, 357-359. doi:10.1038/nmeth.1923
Lawrence, M., Huber, W., Pages, H., Aboyoun, P., Carlson, M., Gentleman, R., Morgan, M. T., Carey, V. J., 2013. Software for computing and annotating genomic ranges. PLoS Comput. Biol. 9, e1003118. doi:10.1371/journal.pcbi.1003118
Le Meur, S., Zinn, M., Egli, T., Thöny-Meyer, L., Ren, Q., 2012. Production of medium-chain-length polyhydroxyalkanoates by sequential feeding of xylose and octanoic acid in engineered Pseudomonas putida KT2440. BMC Biotechnol. 12, 53. doi:10.1186/1472-6750-12-53
Lee, D.-H., Feist, A. M., Barrett, C. L., Palsson, B. Ø., 2011. Cumulative number of cell divisions as a meaningful timescale for adaptive laboratory evolution of Escherichia coli. PLoS One 6, e26172. doi:10.1371/journal.pone.0026172
Li, G.-M., 2008. Mechanisms and functions of DNA mismatch repair. Cell Res. 18, 85-98. doi:10.1038/cr.2007.115
Lim, H. G., Fong, B., Alarcon, G., Magurudeniya, H. D., Eng, T., Szubin, R., Olson, C. A., Palsson, B. O., Gladden, J. M., Simmons, B. A., Mukhopadhyay, A., Singer, S. W., Feist, A. M., 2020. Generation of ionic liquid tolerant Pseudomonas putida KT2440 strains via adaptive laboratory evolution. Green Chem. 22, 5677-5690. doi:10.1039/D0GC01663B
Lim, H. G., Kwak, D. H., Park, S., Woo, S., Yang, J. S., Kang, C. W., Kim, B., Noh, M. H., Seo, S. W., Jung, G. Y., 2019. Vibrio sp. dhg as a platform for the biorefinery of brown macroalgae. Nat. Commun. 10, 2486. doi:10.1038/s41467-019-10371-1
Lim, H. G., Seo, S. W., Jung, G. Y., 2013. Engineered Escherichia coli for simultaneous utilization of galactose and glucose. Bioresour. Technol. 135, 564-567. doi:10.1016/j.biortech.2012.10.124
Linger, J. G., Vardon, D. R., Guarnieri, M. T., Karp, E. M., Hunsinger, G. B., Franden, M. A., Johnson, C. W., Chupka, G., Strathmann, T. J., Pienkos, P. T., Beckham, G. T., 2014. Lignin valorization through integrated biological funneling and chemical catalysis. Proc. Natl. Acad. Sci. USA 111, 12013-12018. doi:10.1073/pnas.1410657111
Liu, Y., Rainey, P. B., Zhang, X.-X., 2015. Molecular mechanisms of xylose utilization by Pseudomonas fluorescens: overlapping genetic responses to xylose, xylulose, ribose and mannitol. Mol. Microbiol. 98, 553-570. doi:10.1111/mmi.13142
Love, M. I., Huber, W., Anders, S., 2014. Moderated estimation of fold change and “dispersion for RNA-seq data with DESeq2. Genome Biol. 15, 550. doi:10.1186/s13059-014-0550-8
Meijnen, J.-P., de Winde, J. H., Ruijssenaars, H. J., 2008. Engineering Pseudomonas putida 512 for efficient utilization of D-xylose and L-arabinose. Appl. Environ. Microbiol. 74, 5031-5037. doi:10.1128/AEM.00924-08
Meijnen, J.-P., de Winde, J. H., Ruijssenaars, H. J., 2009. Establishment of oxidative D-xylose metabolism in Pseudomonas putida S12. Appl. Environ. Microbiol. 75, 2784-2791. doi:10.1128/AEM.02713-08
Meijnen, J.-P., de Winde, J. H., Ruijssenaars, H. J., 2012. Metabolic and regulatory rearrangements underlying efficient D-xylose utilization in engineered Pseudomonas putida S12. J. Biol. Chem. 287, 14606-14614. doi:10.1074/jbc.M111.337501
Mohamed, E. T., Mundhada, H., Landberg, J., Cann, I., Mackie, R. I., Nielsen, A. T., HerrgArd, M. J., Feist, A. M., 2019. Generation of an E. coli platform strain for improved sucrose utilization using adaptive laboratory evolution. Microb. Cell Fact. 18, 116. doi:10.1186/s12934-019-1165-2
Mohamed, E. T., Wang, S., Lennen, R. M., Herrgård, M. J., Simmons, B. A., Singer, S. W., Feist, A. M., 2017. Generation of a platform strain for ionic liquid tolerance using adaptive laboratory evolution. Microb. Cell Fact. 16, 204. doi:10.1186/s12934-017-0819-1
Mohamed, E. T., Werner, A. Z., Salvachúa, D., Singer, C. A., Szostkiewicz, K., Rafael Jiménez-Diaz, M., Eng, T., Radi, M. S., Simmons, B. A., Mukhopadhyay, A., Herrgård, M. J., Singer, S. W., Beckham, G. T., Feist, A. M., 2020. Adaptive laboratory evolution of Pseudomonas putida KT2440 improves p-coumaric and ferulic acid catabolism and tolerance. Metab. Eng. Commun. 11, e00143. doi:10.1016/j.mec.2020.e00143
Nguyen-Vo, T. P., Liang, Y., Sankaranarayanan, M., Seol, E., Chun, A. Y., Ashok, S., Chauhan, A. S., Kim, J. R., Park, S., 2019. Development of 3-hydroxypropionic-acid-tolerant strain of Escherichia coli W and role of minor global regulator yieP. Metab. Eng. 53, 48-58. doi:10.1016/j.ymben.2019.02.001
Nikel, P. I., de Lorenzo, V., 2018. Pseudomonas putida as a functional chassis for industrial biocatalysis: From native biochemistry to trans-metabolism. Metab. Eng. 50, 142-155. doi:10.1016/j.ymben.2018.05.005
Nogales, J., Mueller, J., Gudmundsson, S., Canalejo, F. J., Duque, E., Monk, J., Feist, A. M., Ramos, J. L., Niu, W., Palsson, B. O., 2020. High-quality genome-scale metabolic modelling of Pseudomonas putida highlights its broad metabolic capabilities. Environ. Microbiol. 22, 255-269. doi:10.1111/1462-2920.14843
Peabody, G. L., Elmore, J. R., Martinez-Baird, J., Guss, A. M., 2019. Engineered Pseudomonas putida KT2440 co-utilizes galactose and glucose. Biotechnol Biofuels 12, 295. doi:10.1186/s13068-019-1627-0
Pettersen, E. F., Goddard, T. D., Huang, C. C., Couch, G. S., Greenblatt, D. M., Meng, E. C., Ferrin, T. E., 2004. UCSF Chimera—a visualization system for exploratory research and analysis. J. Comput. Chem. 25, 1605-1612. doi:10.1002/jcc.20084
Phaneuf, P. V., Gosting, D., Palsson, B. O., Feist, A. M., 2019. ALEdb 1.0: a database of mutations from adaptive laboratory evolution experimentation. Nucleic Acids Res. 47, D1164-D1171. doi:10.1093/nar/gky983
Quan, S., Ray, J. C. J., Kwota, Z., Duong, T., Baldzsi, G., Cooper, T. F., Monds, R. D., 2012. Adaptive evolution of the lactose utilization network in experimentally evolved populations of Escherichia coli. PLoS Genet. 8, e1002444. doi:10.1371/journal.pgen.1002444
Reider Apel, A., Ouellet, M., Szmidt-Middleton, H., Keasling, J. D., Mukhopadhyay, A., 2016. Evolved hexose transporter enhances xylose uptake and glucose/xylose co-utilization in Saccharomyces cerevisiae. Sci. Rep. 6, 19512. doi:10.1038/srep19512
Sandberg, T. E., Lloyd, C. J., Palsson, B. O., Feist, A. M., 2017. Laboratory evolution to alternating substrate environments yields distinct phenotypic and genetic adaptive strategies. Appl. Environ. Microbiol. 83. doi:10.1128/AEM.00410-17
Sandberg, T. E., Salazar, M. J., Weng, L. L., Palsson, B. O., Feist, A. M., 2019. The emergence of adaptive laboratory evolution as an efficient tool for biological discovery and industrial biotechnology. Metab. Eng. 56, 1-16. doi:10.1016/j.ymben.2019.08.004
Sandberg, T. E., Szubin, R., Phaneuf, P. V., Palsson, B. O., 2020. Synthetic cross-phyla gene replacement and evolutionary assimilation of major enzymes. Nat. Ecol. Evol. doi:10.1038/s41559-020-1271-x
Saravolac, E. G., Taylor, N. F., Benz, R., Hancock, R. E., 1991. Purification of glucose-inducible outer membrane protein OprB of Pseudomonas putida and reconstitution of glucose-specific pores. J. Bacteriol. 173, 4970-4976. doi:10.1128/jb.173.16.4970-4976.1991
Shen, L., Kohlhaas, M., Enoki, J., Meier, R., Schönenberger, B., Wohlgemuth, R., Kourist, R., Niemeyer, F., van Niekerk, D., Bräsen, C., Niemeyer, J., Snoep, J., Siebers, B., 2020. A combined experimental and modelling approach for the Weimberg pathway optimisation. Nat. Commun. 11, 1098. doi:10.1038/s41467-020-14830-y
Swanson, B. L., Hager, P., Phibbs, P., Ochsner, U., Vasil, M. L., Hamood, A. N., 2000. Characterization of the 2-ketogluconate utilization operon in Pseudomonas aeruginosa PAO1. Mol. Microbiol. 37, 561-573. doi:10.1046/j.1365-2958.2000.02012.x
Udaondo, Z., Ramos, J.-L., Segura, A., Krell, T., Daddaoua, A., 2018. Regulation of carbohydrate degradation pathways in Pseudomonas involves a versatile set of transcriptional regulators. Microb Biotechnol 11, 442-454. doi:10.1111/1751-7915.13263
Wang, L., York, S. W., Ingram, L. O., Shanmugam, K. T., 2019. Simultaneous fermentation of biomass-derived sugars to ethanol by a co-culture of an engineered Escherichia coli and Saccharomyces cerevisiae. Bioresour. Technol. 273, 269-276. doi:10.1016/j.biortech.2018.11.016
Wang, Y., Horlamus, F., Henkel, M., Kovacic, F., Schläfle, S., Hausmann, R., Wittgens, A., Rosenau, F., 2019. Growth of engineered Pseudomonas putida KT2440 on glucose, xylose and arabinose: hemicellulose hydrolysates and their major sugars as sustainable carbon sources. Glob. Change Biol. Bioenergy. doi:10.1111/gcbb.12590
Wargacki, A. J., Leonard, E., Win, M. N., Regitsky, D. D., Santos, C. N. S., Kim, P. B., Cooper, S. R., Raisner, R. M., Herman, A., Sivitz, A. B., Lakshmanaswamy, A., Kashiyama, Y., Baker, D., Yoshikuni, Y., 2012. An engineered microbial platform for direct biofuel production from brown macroalgae. Science 335, 308-313. doi:10.1126/science.1214547
Wehrs, M., Gladden, J. M., Liu, Y., Platz, L., Prahl, J.-P., Moon, J., Papa, G., Sundstrom, E., Geiselman, G. M., Tanjore, D., Keasling, J. D., Pray, T. R., Simmons, B. A., Mukhopadhyay, A., 2019. Sustainable bioproduction of the blue pigment indigoidine: Expanding the range of heterologous products in R. toruloides to include non-ribosomal peptides. Green Chem. 21, 3394-3406. doi:10.1039/C9GC00920E
Wehrs, M., Prahl, J.-P., Moon, J., Li, Y., Tanjore, D., Keasling, J. D., Pray, T., Mukhopadhyay, A., 2018. Production efficiency of the bacterial non-ribosomal peptide indigoidine relies on the respiratory metabolic state in S. cerevisiae. Microb. Cell Fact. 17, 193. doi:10.1186/s12934-018-1045-1
Wong, B. G., Mancuso, C. P., Kiriakov, S., Bashor, C. J., Khalil, A. S., 2018. Precise, automated control of conditions for high-throughput growth of yeast and bacteria with eVOLVER. Nat. Biotechnol. 36, 614-623. doi:10.1038/nbt.4151
Yaegashi, J., Kirby, J., Ito, M., Sun, J., Dutta, T., Mirsiaghi, M., Sundstrom, E. R., Rodriguez, A., Baidoo, E., Tanjore, D., Pray, T., Sale, K., Singh, S., Keasling, J. D., Simmons, B. A., Singer, S. W., Magnuson, J. K., Arkin, A. P., Skerker, J. M., Gladden, J. M., 2017. Rhodosporidium toruloides: a new platform organism for conversion of lignocellulose into terpene biofuels and bioproducts. Biotechnol Biofuels 10, 241. doi:10.1186/s13068-017-0927-5
Yang, J., Zhang, Y., 2015. I-TASSER server: new development for protein structure and function predictions. Nucleic Acids Res. 43, W174-81. doi:10.1093/nar/gkv342
Yu, D., Xu, F., Valiente, J., Wang, S., Zhan, J., 2013. An indigoidine biosynthetic gene cluster from Streptomyces chromofuscus ATCC 49982 contains an unusual IndB homologue. J. Ind. Microbiol. Biotechnol. 40, 159-168. doi:10.1007/s10295-012-1207-9
Zhang, H., Pereira, B., Li, Z., Stephanopoulos, G., 2015. Engineering Escherichia coli coculture systems for the production of biochemical products. Proc. Natl. Acad. Sci. USA 112, 8266-8271. doi:10.1073/pnas.1506781112

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Generation of Pseudomonas putida KT2440 Strains with Efficient Utilization of Xylose and Galactose Via Adaptive Laboratory Evolution

While Pseudomonas putida KT2440 has great potential for biomass-converting processes, its inability to utilize the biomass abundant sugars xylose and galactose has hindered its applications. To address this issue, we utilized Adaptive Laboratory Evolution (ALE) to optimize engineered KT2440 strains in which the Weimberg and Leloir pathways were constructed through heterologous expression of xylD encoding xylonate dehydratase from Caulobacter crescentus and galETKM encoding UDP-glucose 4-epimerase, galactose-1-phosphate uridylyltransferase, galactokinase, and galactose-1-epimerase from Escherichia coli K-12 MG1655. Poor starting strains growth (<0.1 h⁻¹ or none) was optimized via evolution using 3 or 4 independent replicates each to rates of up to 0.25 h⁻¹ on xylose and 0.52 h⁻¹ on galactose. Whole-genome sequencing revealed key mutations in ptxS and kguT encoding a 2-ketogluconate operon repressor and 2-ketogluconate transporter, and gtsABCD encoding an ATP-binding cassette (ABC) sugar transporting system in xylose and galactose growth conditions, respectively. Mutations in the heterologous construct xylD were not observed while mutations in galETKM were observed in one evolution replicate. Key mutations were validated to be causal via reverse engineering, transcriptomic analysis, and growth screens. Finally, we expressed the heterologous indigoidine production pathway in the evolved and unevolved engineered strains and successfully produced 3.2 g/L and 2.2 g/L from 10 g/L of either xylose or galactose in the evolved strains whereas the unevolved strains did not produce any detectable product. Thus, the generated KT2440 strains have potential for broad application as optimized platform chassis to develop efficient microorganism-based biomass-utilizing bioprocesses and ALE was an effective method for optimization of strains with heterologous substrate consumption pathways.

In this study, an ALE approach was applied to engineered KT2440 strains for efficient utilization of two biomass-abundant sugars, xylose and galactose. Initially, we obtained engineered KT2440 strains in which xylD from C. cresentus or galETKM from E. coli (Banerjee et al., 2020) was integrated into the chromosome to construct the Weimberg pathway for xylose utilization or the Leloir pathway for galactose utilization. Then, we evolved the strains in minimal media supplemented with xylose or galactose. While the initial versions grew poorly or did not grow at all, with the ALE approach, we successfully obtained evolved clones that grow on xylose or galactose with higher growth rates (0.25 h⁻¹ on xylose or 0.52 h⁻¹ on galactose). Whole-genome and transcriptome sequencing of evolved isolates revealed key mutational mechanisms that improved sugar utilization. We validated the critical roles of the heterologous genes and commonly mutated genes (kguT and gtsABCD) by deleting them in evolved clones. Finally, we also confirmed their capability to serve as a platform by demonstrating efficient production of indigoidine, a naturally-found blue pigment (Wehrs et al., 2019). Collectively, we expect that the generated strains and related mutational mechanisms will be greatly useful to develop KT2440-based microbial processes for the efficient production of various biochemicals from biomass.

### Methods

Bacterial Cells, Plasmids, and Reagents

Strains and plasmids used in this study are listed in Supplementary Data 1 (filed in U.S. Provisional Patent Application Ser. Nos. 63/168,687, filed on Mar. 31, 2021). Plasmids were constructed by using a NEBuilder HiFi DNA assembly kit from New England Biolabs (NEB, Ipswich, Mass., USA). Oligonucleotides, synthesized by Integrated DNA Technologies (Coralville, Iowa, USA), and template DNA for assembled DNA fragments are listed in Supplementary Data 2 (filed in U.S. Provisional Patent Application Ser. Nos. 63/168,687, filed on Mar. 31, 2021). When high fidelity is required, Q5 High-Fidelity DNA polymerase (NEB); otherwise, OneTaq DNA polymerase (NEB) was used. Plasmids were purified by using a ZymoPURE Plasmid Miniprep kit from Zymo Research (Irvine, Calif., USA). Scar-less genome engineering of the KT2440 strain was conducted by following a conjugation method described in a previous study (Lim et al., 2020). All chemicals were purchased from Sigma Aldrich (St. Louis, Mo., USA) unless mentioned otherwise.

2.2 Cell Cultures

All cell cultures were conducted in an Luria-Bertani (LB, 10 g/L tryptone, 5 g/L yeast extract, 10 g/L NaCl) or phosphate-buffered minimal medium (Lim et al., 2020) unless mentioned otherwise. The minimal medium contains 4 g/L carbon source, 2 g/L (NH₄)₂SO₄, 6.8 g/L Na₂HPO₄, 3 g/L KH₂PO₄, 0.5 g/L NaCl, 2 mM MgSO₄, 0.1 mM CaCl₂), 500 μL/L 2000×trace element solution (Teknova Inc, Hollister, Calif.). The composition of the trace element solution is 4.5 g/L ZnSO₄.7H₂O, 0.7 g/L MnCl₂.4H₂O, 0.3 g/L CoCl₂.6H₂O, 0.2 g/L CuSO₄.2H₂O, 0.4 g/L Na₂MoO₄.2H₂O, 4.5 g/L CaCl₂.2H₂O, 3.0 g/L FeSO₄.7H₂O, 1.0 g/L H₃BO₃, 0.1 g/L KI, 15 g/L disodium ethylenediaminetetraacetate.

Cells were grown in a flask or a microtiter plate. Flask-scale cell cultures were performed with a 30 mL cylindrical tube containing 15 mL of a medium. Seed cultures were prepared by inoculating colonies from LB agar plates into the minimal medium supplemented with 4 g/L glucose. Fully grown cultures were diluted into the fresh medium at the optical density at 600 nm (OD₆₀₀) of 0.1. When OD₆₀₀ reached 0.6-1, cells are harvested and re-inoculated into xylose or galactose supplemented minimal media at OD₆₀₀ of 0.05 to initiate main cultures. Cultures were continuously stirred at 1,100 rpm using a magnetic stirrer and incubated at 30° C. OD₆₀₀ was measured using a Biomate 3S bench-top spectrophotometer from Thermo Fisher Scientific (Waltham, Mass., USA). When a main culture was performed at a small scale, cells were grown on a microtiter plate with a culture volume of 200 μL. A cell culturing plate was continuously shaken with the medium intensity and the cell growth was monitored using an M200 Infinite Pro microplate reader from Tecan (Männedorf, Switzerland).

ALE experiments were conducted in an automated platform (LaCroix et al., 2017; Guzmán et al., 2019) at the flask scale. Multiple colonies of the P. putida strains were picked from LB plates and inoculated independently. Overnight cultures grown on glucose were diluted (1/100) into a fresh medium supplemented with 4 g/L of either xylose or galactose as a carbon source. Once cultures reached the late-exponential phase (OD₆₀₀ of 0.6-1), 150 μL of cultures were passaged iteratively. During a weaning phase, 1 g/L of glucose was additionally supplemented, but its concentration was changed depending on biomass formation (Guzmán et al., 2019).

For the indigoidine production, cells were cultured in 250 mL baffled flasks containing 60 mL of a modified minimal medium where 2 g/L of (NH₄)₂SO₄ was substituted with 100 mM NH₄Cl and the sugar concentrations were increased to 10 g/L. The flasks were shaken at 200 rpm and 30° C. 3 g/L of arabinose was included to induce the expression of the indigoidine synthetic genes (Banerjee et al., 2020) at 0 h.

Genome and Transcriptome Sequencing

For genome sequencing, intermediate and endpoint clones were grown in LB media and cell pellets were harvested after overnight cultures. Genomic DNA samples were prepared by using a Quick-DNA Fungal/Bacterial Miniprep kit from Zymo Research. Sequencing library samples were prepared by using a Nextera XT kit from Illumina (San Diego, Calif., USA) by following manufacturer's protocol. Raw sequencing reads were obtained by using a NovaSeq 6000 (illumina) at the UC San Diego IGM Genomics Center and analyzed by using Breseq (version 0.33.1) (Deatherage and Barrick, 2014) and Bowtie2 (version 2.3.4.1) (Langmead and Salzberg, 2012). Mutations were detected by comparing genome sequences of evolved clones and a respective starting strain. Outputs from the analytical software were processed by an in-lab pipeline and uploaded to ALEdb v1.0 (Phaneuf et al., 2019). Genome sequencing raw files are listed in Supplementary Data 3 and available at the NCBI SRA database (BioProject number: PRJNA682829).

For transcriptome sequencing, RNA samples were prepared by using grown cells in the minimal medium supplemented with xylose or galactose as a sole carbon source. Cells at the exponential phase (OD₆₀₀ 0.4-0.6) were treated with an RNAprotect Bacteria Reagent from Qiagen. Total RNA was extracted by using a Quick-RNA Fungal/Bacterial Miniprep kit from Zymo Research (Irvine, Calif., USA). Remaining genomic DNA and ribosomal RNA were removed by following a previously reported method (Lim et al., 2020) that uses RNase-free DNase I (NEB), thermostable RNase H (Lucigen, Middleton, Wis., USA), and oligonucleotides designed to bind the ribosomal RNA of KT2440 specifically. Paired-end libraries were prepared by using a KAPA RNA HyperPrep kit from Kapa Biosystems (Wilmington, Mass., USA) and sequenced using the NovaSeq 6000. Raw sequencing files were processed using Bowtie2 (Langmead and Salzberg, 2012) and summarizeOverlaps (Lawrence et al., 2013). Differentially expressed genes were detected by using DEseq2 (Love et al., 2014). Transcriptome sequencing raw files are available at Gene Expression Omnibus (GEO, accession number: GSE155767). The transcriptome of the wildtype strain cultivated on glucose (GEO accession number: GSE149827) was used as the reference condition (Lim et al., 2020).

2.4 Biomass and Metabolite Quantification

Cell biomass was determined by converting OD₆₀₀ to a dry cell weight (DCW)/L using a conversion factor of 0.38 (Dvořák and de Lorenzo, 2018). Metabolite concentrations were quantified by using a 1260 Infinity II LC system (Agilent, Santa Clara, Calif., USA) equipped with an HPX-87H (Biorad, Hercules, Calif., USA). 5 mM H2SO₄ was used as a mobile phase at a flow rate of 0.5 mL/min. The column temperature was maintained at 45° C. Refractive index signals were analyzed.

Indigoidine was quantified by following a previously reported method (Banerjee et al., 2020; Yu et al., 2013). Briefly, 100 μL of cultures were centrifuged at 15,000 rpm for 2 min and the supernatant was discarded. Subsequently, 500 μL dimethyl sulfoxide (DMSO) was added to the cell pellet and the mixture was vigorously vortexed for 10 min to extract indigoidine. The absorbance of 100 μL of indigoidine-dissolved DMSO was measured at 612 nm by using a microtiter plate reader from BD Biosciences (Molecular Devices, CA, USA). The absorbance was converted to the concentration by using an equation of Y (mg/L of indigoidine)=240.31×A₆₁₂−2.1005. The standard curve was generated using indigoidine harvested from a xylose ALE P. putida strain with the indigoidine production pathway and grown in M9 media supplemented with 10 g/L xylose. Indigoidine was isolated and validated for purity by H-NMR as described previously (Banerjee et al., 2020). The theoretical maximum yield from xylose was calculated as 0.74 g indigoidine/g xylose using the KT2440 genome scale metabolic model iJN1462 (Nogales et al., 2020) and this was extended to account for xylose utilization through the Weimberg pathway using flux balance analysis. The yield from galactose (0.77 g indigoidine/g galactose) was adapted from a previous study (Banerjee et al., 2020).

2.5 Protein Structure Prediction and Visualization

Protein structures were predicted by using the I-TASSER software (Yang and Zhang, 2015). Predicted structures were visualized by using the UCSF Chimera (Pettersen et al., 2004).

### Results

3.1 Adaptive Laboratory Evolution of Engineered P. putida Strains for Xylose and Galactose Utilization Enhances Catalytic Capabilities

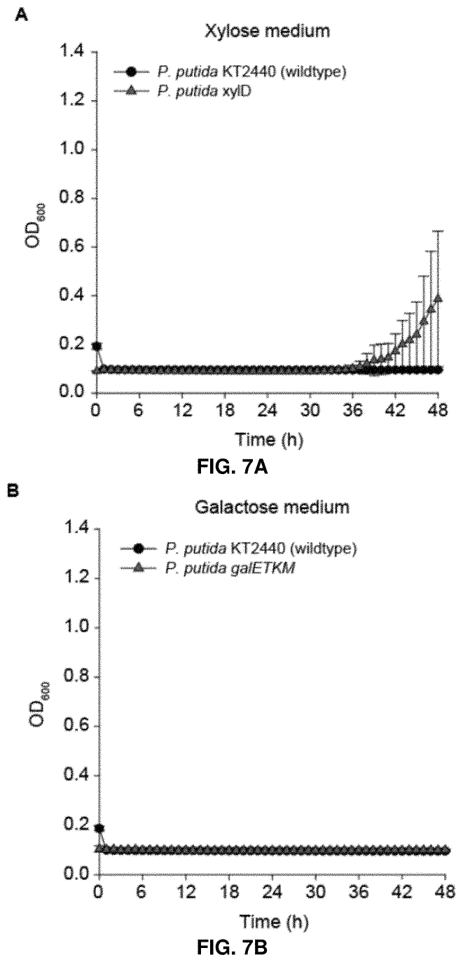

The initial growth characteristics of two engineered KT2440 strains (P. putida xylD and P. putida galETKM, Supplementary Data 1) in minimal media supplemented with either xylose or galactose as a sole carbon source were characterized. The P. putida xylD strain was engineered to express the xylD gene encoding xylonate dehydratase from C. crescentus (Meijnen et al., 2009) to construct the Weimberg pathway for xylose utilization. xylD was expressed from the chromosome under a constitutive promoter and 5′-untranslated region (Supplementary Note 1). Consistent with previous observation (Meijnen et al., 2009), the expression of xylD enabled growth of the strain on xylose as a sole carbon source (FIG. 7A); however, it showed a relatively low growth rate (μ, ˜0.07 h⁻¹), long lag time (>36 h), and clumping (data not shown) during initial growth characterizations. For the second starting strain, we utilized a previously constructed P. putida galETKM strain (Banerjee et al., 2020), in which the native galactose operon (galETKM) of E. coli K-12 MG1655 was introduced into the chromosome to construct the Leloir pathway. This P. putida galETKM starting strain did not display observable growth on galactose minimal media during a 48 h culture screen (FIG. 7B); the strain did grow under a rich medium or glucose minimal media condition. These initial screens set the starting parameters for a multifaceted ALE strategy to improve the sugar utilization of both KT2440 strains on their respective sole carbon source sugars.

ALE experiments with two different strategies were conducted to optimize growth rates and catalytic activity on the targeted xylose and galactose sugars. Given that the P. putida xylD strain could initially grow on xylose, we continuously propagated the strain in a constant condition ALE on an automated platform (LaCroix et al., 2015) on minimal medium supplemented with xylose (4 g/L) as the sole carbon source (FIG. 1A). For the P. putida galETKM starting strain, which could not initially grow solely on galactose within 48 hours, we propagated the strain in a minimal medium containing 4 g/L galactose and the additional supplement of 1 g/L glucose to support cell growth and subsequent mutation accumulation (FIG. 1B); this complementary approach was also automated and adapted from Guzman et al (Guzmán et al., 2019). The supplemented culture (hereafter referred to as the main culture) was regularly screened by inoculating it into a fresh galactose only minimal medium (hereafter referred to as the test culture). If cells failed to produce an observable grow rate during 48 h in the test culture, it was discarded, and the main culture was continued. Once a stable growth rate (μ>0.05 h⁻¹) in a test culture was observed for a given replicate after several generations, this culture was continued as a constant condition galactose only culture and continued for growth rate selection until the end of the experiments. It should be noted that in the original main culture line, the amount of glucose was gradually decreased depending on the final biomass density during stationary phase as previously described (Guzmán et al., 2019). Both strategies of automated ALE experiments were parallelly conducted with four independent biological replicates (Table 3).

TABLE 3
Summary of the ALE experiments.

| | | | | # of | # of | | | |
|---|---|---|---|---|---|---|---|---|
| | | | | passages with | passages with | | | Growth rate of |
| | | | Initial | additional | xylose or | | Cumulative | the end-point |
| | Target | Starting | growth rate | glucose | galactose | # of | cell divisions | population |
| ALE # | sugar | strain | (h⁻¹) | supplementation | only | generations | (CCD, 10¹¹) | (h⁻¹) |
| ALE1 | Xylose | P. putida | 0.07 | n.a. | 37 | 203 | 11.5 | 0.22 |
| ALE2 | | xylD | | n.a. | 72 | 368 | 29.3 | 0.25 |
| ALE3 | | | | n.a. | 76 | 398 | 30.5 | 0.22 |
| ALE4 | | | | n.a. | 56 | 313 | 22.0 | 0.23 |
| ALE5 | Galactose | P. putida | n.a. | 35 | 85 | 474 | 22.5 | 0.33 |
| ALE6 | | galETKM | | 36 | 90 | 466 | 23.9 | 0.46 |
| ALE8 | | | | 44 | 92 | 479 | 22.5 | 0.52 |

n.a.: not applicable

The ALE experiments were conducted for approximately three months (203-479 generations, equivalent to 11.5-30.5×10¹¹ cumulative cell divisions) and the growth rates of populations during the exponential phase were continuously monitored. Notably, the growth rates of all independent replicates of P. putida xylD populations were significantly increased at an early stage of the experiments (ALE1-4 and FIG. 1C). However, no further significant increases were observed; the final growth rates of populations were between 0.22 and 0.25 h⁻¹. In the case of the P. putida galETKM strain (FIG. 1D), successful growth on solely galactose was observed in three replicates (ALE5, ALE6, and ALE8). The passage numbers before displaying the stable growth varied between 35-44 flasks (Table 3). Although the initial growth rates of these novel populations were relatively low (approximately 0.1 h⁻¹), the rates were gradually increased over time and reached between 0.33 h⁻¹ and 0.52 h⁻¹, indicating that the evolved strains acquired beneficial mutations which improved galactose catabolism.

Clones were isolated from each experiment at several time points and validated to display the observed phenotypes from the evolved populations. Two or three intermediate evolutionary time points for ALE 1-4 and four points for ALE 5, 6, 8 (FIG. 1C and FIG. 1D) were isolated and their specific growth rates during exponential growth were measured in the xylose or galactose minimal medium, respectively (FIG. 1E and FIG. 1F). For clones derived from the xylD starting strain, the growth rates of isolates generally corresponded to the growth rates of populations from which the strains were isolated; they were in a range of 0.20 h⁻¹-0.26 h⁻¹, regardless of isolation timepoints. Conversely, the growth rates of evolved P. putida galETKM clones from later evolutionary time populations were greater than those isolated from early populations; the end-point isolates showed the growth rates between 0.35 h⁻¹ and 0.52 h⁻¹.

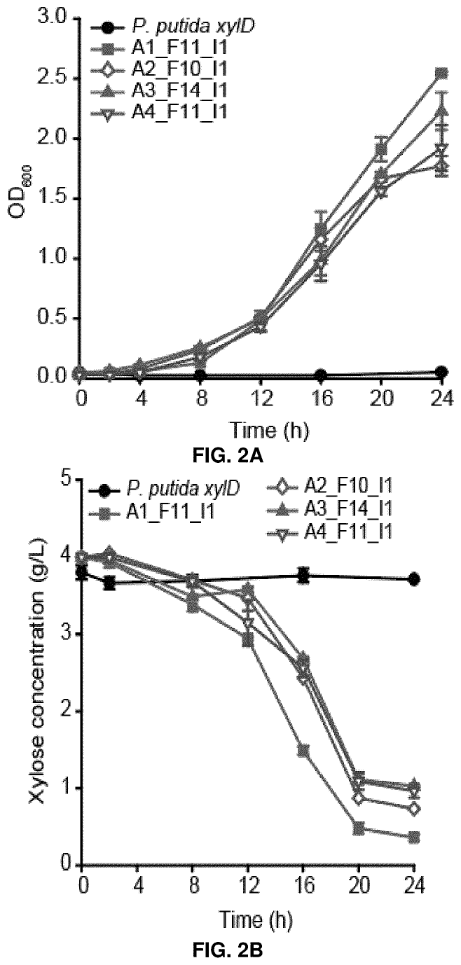

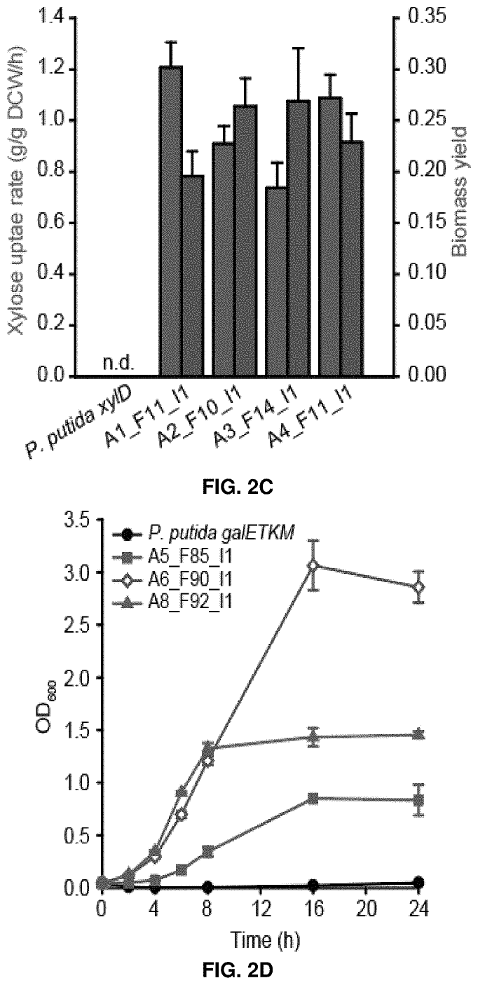

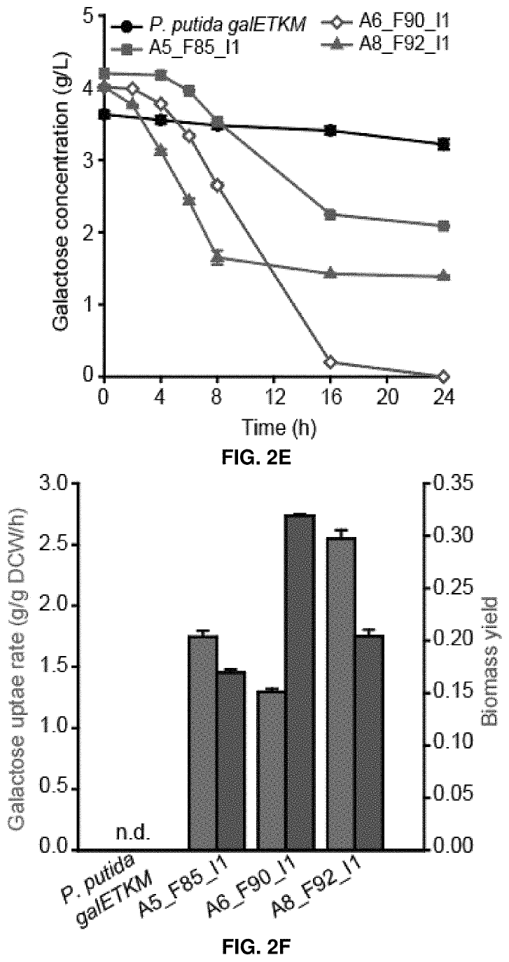

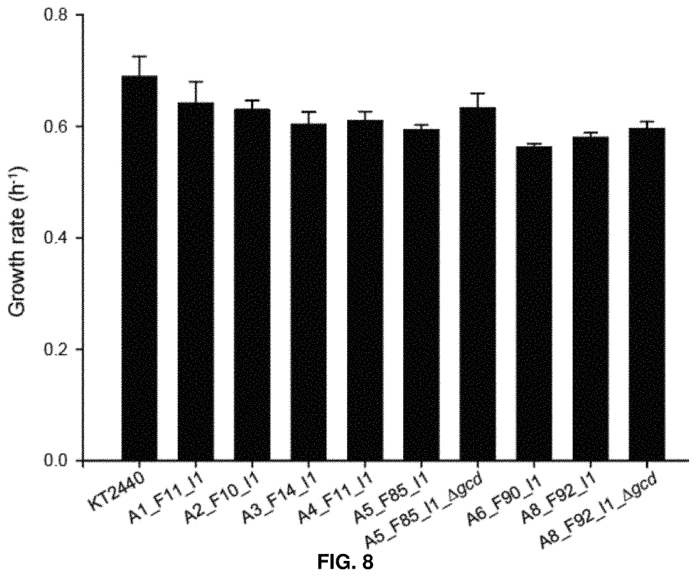

Growth, sugar consumption, and biomass yields were determined in detail to physiologically characterize evolved strains and compare them to the starting strains (FIG. 2A to FIG. 2F). The earliest isolated P. putida xylD clones (AL_F11_I1, A2_F10_I1, A3_F14_I1, and A4_F11_I1) from first-time points and the end-point P. putida galETKM clones (A5_F85_I1, A6_F90_I1, and A8_F92_I1) were selected as representative clones for in depth physiological characterization. While the starting strains showed no growth with the consumption of a negligible or small amount of xylose or galactose, the evolved strain demonstrated greatly improved growth and sugar consumption. The four evolved P. putida xylD clones showed similar growth and sugar consumption profiles (FIG. 2A and FIG. 2B). Among them, their xylose uptake rates varied by a 1.6-fold (FIG. 2A to FIG. 2C); the A1_F11_I1 strain showed the highest uptake catalytic rate (1.2 g xylose g DCW⁻¹ h⁻¹), but the lowest biomass yield (0.20 g xylose g DCW⁻¹). Interestingly, the three isolated evolved P. putida galETKM clones showed noticeably different growth and sugar consumption profiles (FIG. 2D to FIG. 2F). Only the A6_F90_I1 strain fully grew and consumed the provided 4 g/L galactose in 24 h of culturing at the lowest uptake rate of 1.3 g galactose g DCW⁻¹ h⁻¹ of all isolated clones. While the sugar uptake rates of the other P. putida galETKM clones were 1.3- and 2.0-fold higher, these two strains did not fully consume galactose and growth ceased after reaching OD₆₀₀ of 0.85 or 1.2, resulting in two- or three-times less the final biomass density and yield. Additionally, the growth rates of the all characterized clones on glucose remained at similar levels compared to the wild-type strain (FIG. 8). While there are several differences, all isolates showed significantly improved sugar (i.e., xylose and galactose) consumption capabilities when compared to the starting strains, confirming that the ALE strategy was successful to generate strains with enhanced catalytic activity for both xylose and galactose utilization.

3.2 Genomic and Transcriptomic Sequencing Elucidates Causal Mutations and their Impact on Enhanced Catalytic Phenotypes

To identify beneficial mutations in the engineered and evolved strains on xylose and galactose, we conducted whole-genome sequencing of clones isolated during the middle (i.e., intermediate) and end of the evolutions (i.e., endpoints). Furthermore, the transcriptomes of a set of selected clones were measured and analyzed to understand the effect of mutations and resulting transcriptional changes in the different carbon source culturing environments.

3.2.1. Whole-Genome Sequencing Revealed Mutations on Transport and Catabolic Processes

Whole genome sequencing of eleven P. putida xylD and twelve P. putida galETKM evolved isolates successfully revealed several genes or regions (hereafter, referred as regions) commonly mutated across the independently evolved replicates (Supplementary Data 3, FIG. 2A and FIG. 2B). Surprisingly, mutations in six (ptxS, kguT, gacS, ftsH, PP_4173, and galP-I/PP_1174) and three (gtsABCD, oprB-1I/yeaD, and oprB-II) regions accounted for 44% (38 out of 86) and 32% (20 out of 63) of the total mutations in P. putida xylD and P. putida galETKM isolates, respectively (Table 1). Although we expected frequent mutations in the heterologous genes, given that heterologous gene expression cassettes have been mutation targets in previous heterologous pathway optimization studies (Elmore et al., 2020; Sandberg et al., 2020), only the galETKM region was mutated in isolates from ALE 8 where a single amino acid change mutation in galK and single nucleotide change in the intergenic region between galE and its neighboring gene, prfC (Supplementary Data 3) were observed; no mutations occurred in the xylD region. Furthermore, it should be noted that the A4_F56_I1 and A6_F90_I1 clones acquired mutations in mutS, encoding a mismatched DNA repair protein (Li, 2008). In particular, a relatively higher number of mutations (42 mutations) were identified in the former strain when compared to mutation numbers of other isolates (up to 10 mutations).

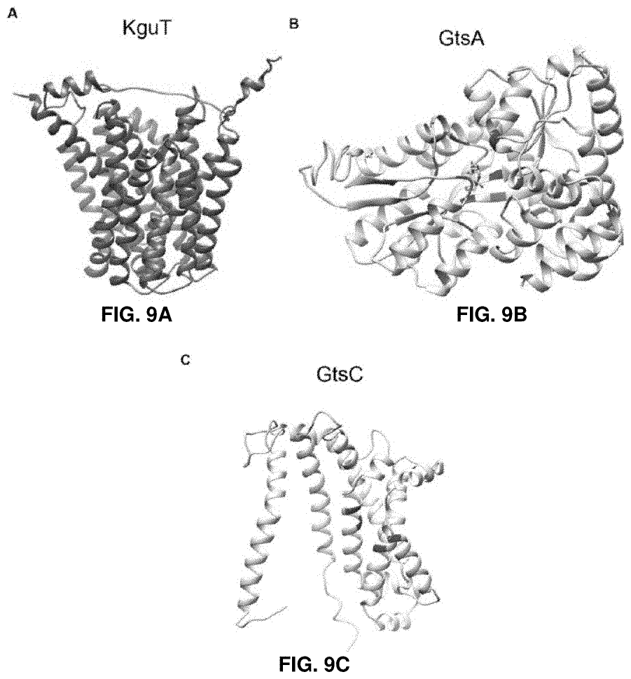

The two genes ptxS and kguT, responsible for one of three peripheral glucose utilization pathways (FIG. 2C), were highly mutated in all eleven sequenced P. putida xylD isolates (100% for ptxS and 82% for kguT) suggesting their critical roles for the improved xylose utilization (FIG. 2A). It is known that ptxS encodes a LacI-family transcription factor, namely a 2-ketogluconate utilization repressor. The binding of 2-ketogluconate mediates the dissociation of PtxS from the promoter region of the kguEKT-ptxD operon (del Castillo et al., 2008; Swanson et al., 2000; Udaondo et al., 2018). Considering that mutations which altered the DNA binding motif (R30S, S29F, or V28F) and partial deletions (Δ51 or Δ438 bp) of PtxS were identified in the isolates, it implies that these mutations de-repressed the expression of kguE, kguK, kguT, and ptxD and as a result, enabled an increase in xylose catabolism. Mutations found in KguT were mostly SNPs (seven unique SNPs found overall) which effected single amino acids and a small deletion or change of a few amino acids at the end of the protein (FIG. 9A), likely effecting the transportation of xylonate. Interestingly, it was found that the A2_F10_I1 and A3_F14_I1 strains with relatively lower xylose uptake rates (FIG. 2C) did not acquire any mutations in KguT. Thus, an association can be drawn between major xylose catabolic and growth rate improvements and mutations in ptxS and kguT simultaneously in the strains examined.

Additional genetic regions were independently mutated in two or more xylD ALE experiments (Table 1), in addition to the ptxS and kguT regions, at a lower frequency. These regions were gacS (36%), ftsH (27%), PP_4173 (27%), and galP-I/PP_1174 (the intergenic region between galP-1 and PP_1174, 18%). Mutations in gacS (encoding a sensor protein, GacS) and PP_4173 (encoding a two-component system sensor histidine kinase/response regulator) were also found in previous ALE studies examining tolerance to different compounds, but with a similar base media and growth environment (Lim et al., 2020; Mohamed et al., 2020). Thus, the gacS and PP_4173 mutations appear to be related to general adaptation to the media or culturing environment used in this study. However, the mutations in ftsH (encoding an Integral membrane ATP-dependent zinc metallopeptidase) and galP-I (encoding a porin-like protein)/PP_1174 (encoding a hypothetical protein) have not been previously identified in an ALE study and it is unclear how they relate to the specific xylose utilization phenotype.

TABLE 1
Commonly mutated genes and regions in evolved strains.

| | | | |
|---|---|---|---|
| | | | Number |
| | | Frequency | of unique |
| Region | Function | (total samples) | mutations |

P. putida xylD n = 11

| | | | |
|---|---|---|---|
| ptxS | Ketogluconate | 100% (ALE1-4) | 5 |
| | utilization operon | | |
| | repressor | | |
| kguT | 2-Ketogluconate | 82% (ALE1-4) | 9 |
| | transporter | | |
| gacS | Sensor protein | 36% (ALE2 and ALE3) | 3 |
| ftsH | Integral membrane | 27% (ALE2 and ALE4) | 3 |
| | ATP-dependent zinc | | |
| | metallopeptidase | | |
| PP_4173 | Two-component | 27% (ALE1 and ALE2) | 3 |
| | system sensor | | |
| | histidine | | |
| | kinase/response | | |
| | regulator | | |
| galP-I/ | Porin-like protein/ | 18% (ALE2 and ALE4) | 2 |
| PP_1174 | hypothetical protein | | |

P. putida galETKM n = 12

| | | | |
|---|---|---|---|
| gtsABCD | Mannose/glucose | 66% (ALE5, ALE6, | 7 |
| | ABC transporter | ALE8) | |
| oprB-I/ | Carbohydrate-selective | 58% (ALE5, ALE6, | 4 |
| yeaD | porin-I/glucose-6- | ALE8) | |
| | phosphate 1-epimerase | | |
| oprB-II | Carbohydrate- | 17% (ALE5 and ALE8) | 1 |
| | selective porin-II | | |

In the case of evolved P. putida galETKM isolates, the three commonly mutated regions were related to the transport of glucose (Table 1), implying galactose transportation was the major bottleneck. Most importantly, mutations in the gtsABCD region were observed in all three endpoint isolates as well as many intermediate isolates (66%, FIG. 3B). Clear growth rate increases were observed after acquiring one of these mutations (F2→F21 in ALE5, F13→F39 in ALE6, F1→F38 in ALE8, FIG. 1E and FIG. 1). These genes encode an ATP-binding cassette (ABC) sugar transporting system, consisting of a sugar-binding protein (GtsA), two subunits of an ABC transporter (GtsB and GtsC), and an ATP binding protein (GtsD); this system is known to transport glucose into the cytosol through the inner membrane (del Castillo et al., 2008). All endpoint clones acquired mutations in gtsA (A100V, N304D, N304S, and A427V) and mutations in gtsC (F122L, L133F, and T238I) were observed in only clones from ALE8 (FIG. 9B and FIG. 9C). Previously, it was observed that mutations in gtsABCD allowed the transportation of xylose (Meijnen et al., 2012), indicating its promiscuity in transporting other compounds. Similarly, these frequent mutations in the gtsABCD region strongly suggested that the major bottleneck was the transportation of galactose into the cytosol. In addition to gtsABCD, two regions also related to glucose metabolism (oprB-1I/yeaD and oprB-II) were also commonly mutated (FIG. 3B). OprB porins are known to transport glucose into the periplasm (del Castillo et al., 2008; Saravolac et al., 1991). Mutations in oprB-I (encoding carbohydrate-selective porin-I)/yeaD (encoding glucose-6-phosphate 1-epimerase) regions were a stop codon insertion and frameshift in oprB-I, or single nucleotide mutation in their intergenic region. Given that two oprB-I mutations are loss of function mutations, it was inferred that its mutation was not beneficial for galactose utilization. In oprB-II, a silent mutation (Y265Y) identically occurred in two endpoint isolates from ALE5 and ALE8 (FIG. 3B) which displayed relatively higher galactose consumption rates compared to the endpoint isolate from ALE6 (FIG. 2F). The same mutation was also observed in previous evolution studies with glucose (Lim et al., 2020; Mohamed et al., 2020), likely suggesting that it improves the transport of hexoses into the cytosol.

3.2.2 Transcriptome Analysis of Evolved Clones Confirms the Xylose and Galactose Utilization Pathways

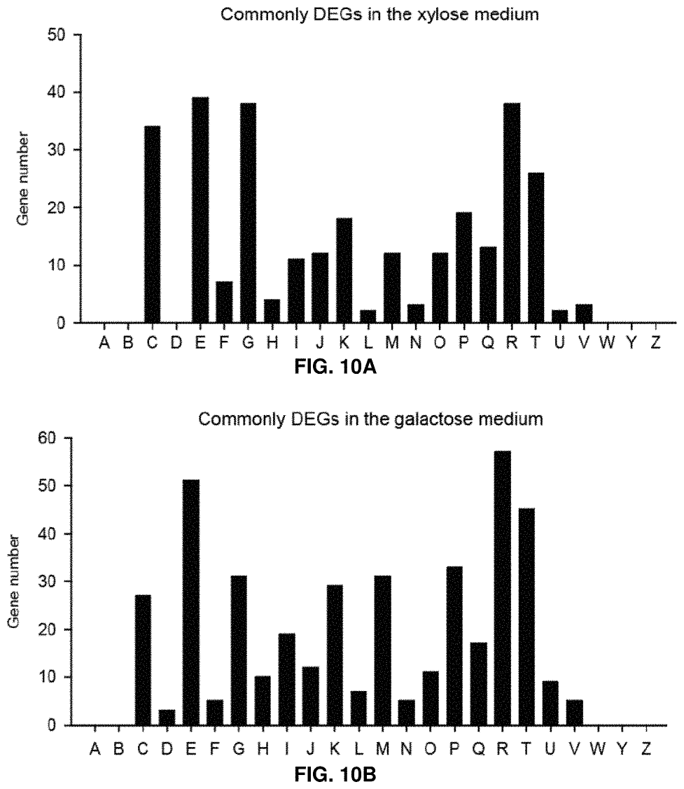

To further examine the impact of mutations on transcriptional changes in the context of xylose and galactose utilization, the transcriptomes of representative clones were analyzed via RNA-Seq and compared with that of the wildtype KT2440 strain growing on glucose (Lim et al., 2020). The transcriptomic analysis revealed large numbers (from 890 to 1,857) of differentially expressed genes (DEGs), indicating changing carbon sources induces global responses (Table 4 and Supplementary Data 3). Among them, in the xylose medium, 598 genes were common DEGs across the P. putida xylD derived strains and related to amino acid metabolism, carbohydrate metabolism and transport while 895 genes were common DEGs across the P. putida galETKM derived strains and related to amino acid metabolism and signal transduction (FIG. 10).

TABLE 4
Numbers of differentially expressed genes in evolved isolates^{a, b}

| | Xylose | | | | Galactose | | |
|---|---|---|---|---|---|---|---|
| | A1_F11_I1 | A2_F10_I1 | A3_F14_I1 | A4_F11_I1 | A5_F85_I1 | A6_F90_I1 | A8_F92_I1 |
| Up | 420 | 505 | 561 | 554 | 586 | 794 | 505 |
| Down | 470 | 604 | 779 | 488 | 844 | 1,063 | 909 |
| Total | 890 | 1,109 | 1,340 | 1,042 | 1,430 | 1,857 | 1,414 |

^{a}Transcriptomes of each strain grown on xylose or galactose were compared with that of the wildtype KT2440 grown on glucose.
^{b}The xylD and galETKM genes were excluded from the counting.

To directly investigate the effect of mutations and utilization of different sugars on carbon metabolism, we focused on the expression level changes of endogenous genes in central carbon metabolism (FIG. 3C and FIG. 3D). In the analyzed four evolved P. putida xylD isolates, indeed, the expression levels of ptxS, kguETK, and ptxD genes were highly up-regulated, likely due to the derepression by PtxS (FIG. 3D). Especially, the expression level of kguT was increased by 20.3-fold, on average, when compared to that of the wildtype grown on glucose. Although a previous study suggested that gntT is responsible for the transportation of xylonate (Meijnen et al., 2009), its expression level was hugely decreased, suggesting that xylonate is transported by KguT, and not GntT. Additionally, it was observed that the expression levels of the PP_2834-2837 genes were significantly increased (fold changes >500). One of these genes, PP_2835, was previously suggested to encode 2-keto-3-deoxy-xylonate dehydratase (Meijnen et al., 2009) due to its high structural similarity with that of C. crescentus. The upregulation of PP_2835 supports the hypothesis on its role to enable xylose metabolism via the Weimberg pathway. In addition to up-regulated genes, there were also noteworthy down-regulated genes; given xylose directly enters the TCA cycle after its conversion to α-ketoglutarate, genes related to glucose metabolism was relatively down-regulated.

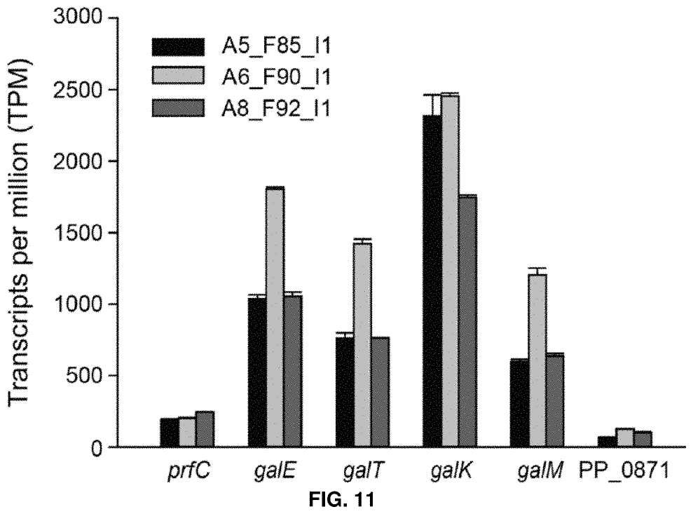

The expression levels in the three evolved P. putida galETKM were also investigated to confirm the galactose utilization pathway and to investigate transcriptional changes of three mutated regions (gtsABCD, oprB-1I/yeaD, oprB-II, FIG. 3D). As expected, the expression levels of gtsABCD were highly up-regulated (up to 9-fold) in the three isolates (FIG. 3D), supporting that they are closely related to the improved galactose utilization. The expression levels of oprB-I, in which perceived loss-of-function mutations occurred, were commonly increased (5.3-fold on average) while the yeaD expression levels changed inconsistently at less extents. Notably, the expression levels of oprB-II were indeed increased in the two endpoint isolates with the Y265Y mutation in this gene (the A5_F85_I1 and A8_F92_I1 strains) by a 13.8-fold and a 9.7-fold (FIG. 3B) whereas the level in the A6_F90_I1 without the mutation decreased by a 5.0-fold. This observation supports that this synonymous mutation, upregulates its expression; previously, changed gene expression levels by synonymous mutations have been observed in Pseudomonas species (Bailey et al., 2014). Furthermore, it was likely that this mutation also affected the expression of a downstream gene, gcd, as its levels were similarly changed (5.6-fold and 6.8-fold increases in the two strains with the mutation and a 3.7-fold decrease in the other strain). The effect of gcd was further evaluated in a targeted analysis (see below) Additionally, we compared the expression levels of the galETKM genes and their neighboring PP_0871 and prfC genes (FIG. 11) to investigate potential transcriptional changes caused by the two mutations in the A8_F92_I1 stain (Supplementary Data 3). However, the levels in A8_F92_I1 strain were generally similar to those in A5F85_I1, which do not have any mutations in this region, indicating that they do not significantly affect the transcription levels.

3.3 Reverse Engineering Validates the Xylose and Galactose Utilization Pathways and Phenotypes

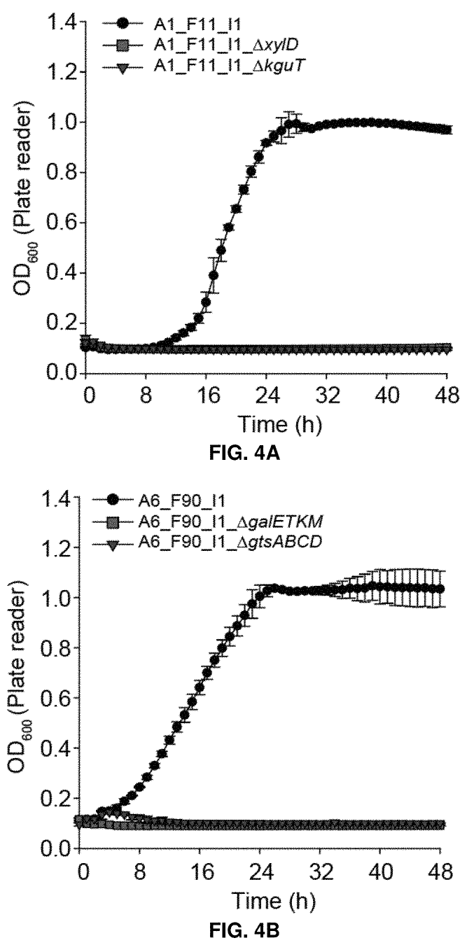

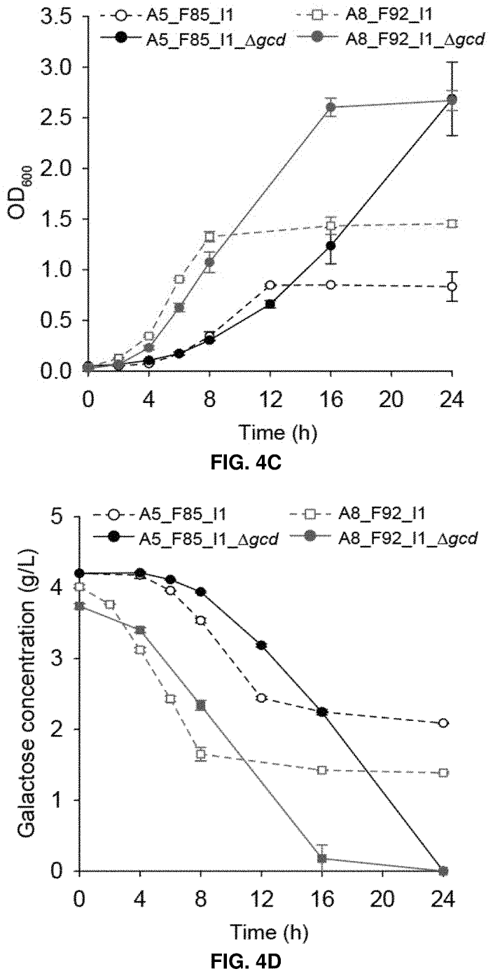

The essentiality of the heterologous and mutated genes was examined via gene deletions and subsequent growth measurements on the constructed clones. Initially, the heterologous xylD or galETKM was deleted in the A1_F11_I1 strain and A6_F90_I1 strain, respectively. Unsurprisingly, the A1_F11_I1_ΔxylD and A6_F90_I1_ΔgalETKM strains completely lost their capability to grow on xylose or galactose (FIG. 4A and FIG. 4B), respectively, confirming the essentialities of these genes. It is worthwhile to note that despite the presence of an endogenous fucD encoding fuconate dehydratase (which was significantly overexpressed by on average 62.6-fold, FIG. 3D) that this potential promiscuous activity could not support xylose-based growth in the strain. Next, we validated the roles of two frequently mutated genes (kguT and gtsABCD). As expected, the A1_F11_I1_ΔkguT and A6_F90_I1_ΔgtsABCD strains lacking the mutated transporter could not grow on xylose or galactose, respectively, confirming that these sugar transporters are indeed critical to the sugar metabolism. Collectively, these results indicate that xylose and galactose are utilized via the introduced Weimberg and Leloir pathways, respectively, and KguT and GtsABCD are essential for the respective sugar metabolism.

The effect and removal of gcd was evaluated in evolved strains given its significant upregulation and its known role to generate growth-inhibiting dead-end byproducts due to its broad substrate specificity (Dokter et al., 1987; Dvořák and de Lorenzo, 2018; Shen et al., 2020). Clones A5_F85_I1 and A8_F92_I1 were evaluated using a deletion of gcd for this analysis as their growth was arrested before full galactose consumption during phenotypic characterization (FIG. 2E). Indeed, after the deletion of gcd, the two strains (A5_F85_I1_Δgcd and A8_F92_I1_Δgcd) showed significantly increased biomass formation and galactose consumption (FIG. 4C). The final OD₆₀₀ reached 2.69 and 2.67 and 4 g/L galactose was fully consumed during 24 h, respectively. Galactose uptake rates were 0.85 g/g DCW/h and 1.61 g/g DCW/h, respectively, which is a slight reduction versus their parent strains over the same time window. Overall, these results confirmed that gcd overexpression induced stunted growth of the two evolved isolates and this phenomena was not observed in clone A6_F90_I1 which did not possess a mutation in the neighboring oprB-II.

3.4. Production of Indigoidine with the Evolved Isolates

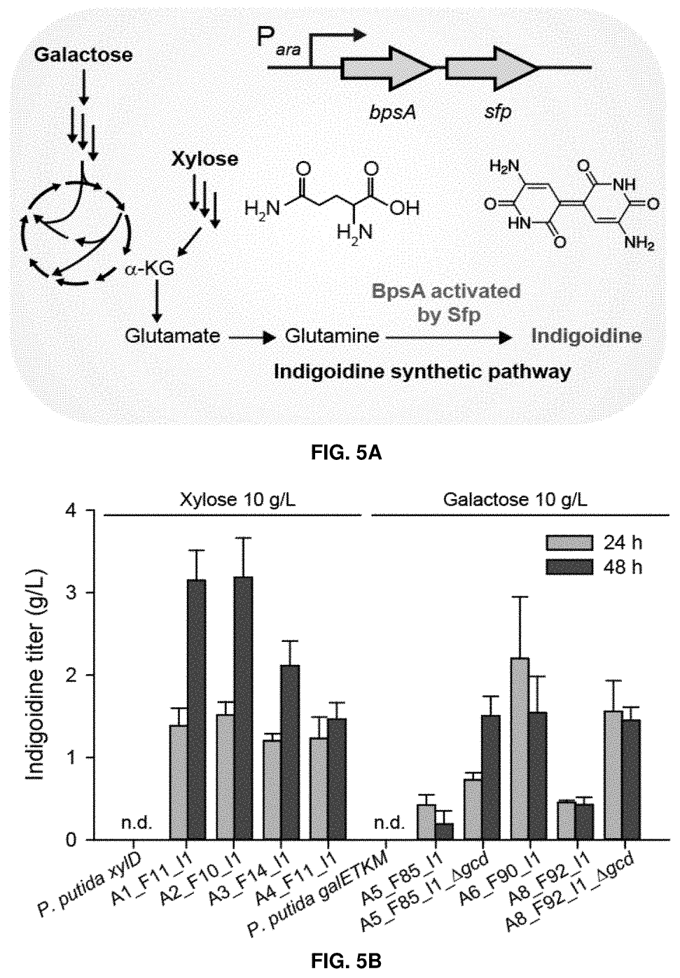

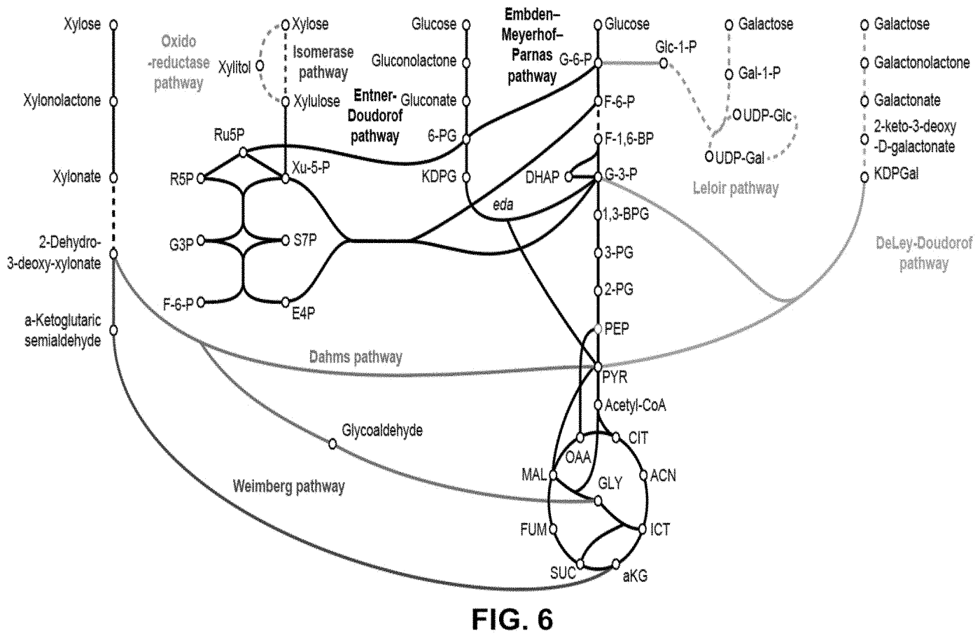

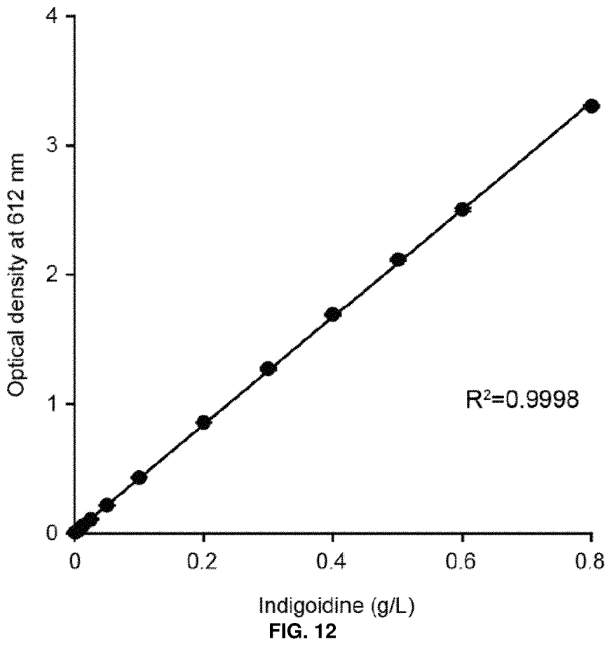

The applicability of the evolved strains and further gcd-deleted strains as preferred host chassis for the biochemical production of indigoidine from either xylose or galactose was examined. In particular, the Δgcd strains were included, given the deletion did not significantly affect the growth rates on glucose (FIG. 8). For this demonstration, we introduced the production pathway of indigoidine, which is a natural pigment and has an industrial interest (Banerjee et al., 2020; Wehrs et al., 2019, 2018), in evolved isolates (FIG. 5A). It was previously shown that indigoidine can be produced by the heterologous expression of bpsA encoding blue pigment synthetase A from Streptomyces lavendulae and sfp encoding 4′-phosphopantetheinyl transferase from Bacillus subtilis (Banerjee et al., 2020; Wehrs et al., 2019). We expressed the two genes under the arabinose-inducible promoter and integrated into the genome (see Methods). Subsequently, the resulting strains were cultivated in minimal media supplemented with 10 g/L xylose or galactose. The cultivation showed that all evolved strains with the heterologous production pathway successfully produced indigoidine whereas the starting strain with the pathway did not produce a detectable amount of indigoidine (FIG. 5B and FIG. 12). The titers varied, on average 2.5±0.8 g/L for xylose and 0.8±0.5 g/L for galactose at 48 h, depending on the sugar source and host, indicating the use of different sugars and host genotypes significantly affect the production. Specifically, xylose utilization via the Weimberg pathway allowed much higher indigoidine titers (3.1-fold on average) compared to the galactose utilization via the Leloir pathway. Among them, the A2_F10_I1_indigo strain produced 3.2 g/L, which is higher value than the titer (1.5-2 g/L) from the same amount of glucose (Banerjee et al., 2020). The titer of 2.2 g/L achieved by A6_F90_I1 strain was also comparable, while the other two strains did not show high titers, probably due to the stalled growth and galactose consumption (FIG. 4A to FIG. 4D). Both Δgcd strains showed improved indigoidine production compared to their parental strains, but the titers were still less than the titer achieved with the A6_F90_I1 strain. The highest titers for each carbon source represent up to 43% and 29% of the maximum theoretical production (see Methods for calculation). Collectively, this successful demonstration of the indigoidine production supports the applicability of the evolved clones in various biochemical production processes as optimized chassis when compared to the initial engineered but unevolved counterparts.

### DISCUSSION AND CONCLUSION

To develop economically feasible bioprocesses, it is essential to utilize host microorganisms that efficiently utilize carbon sources available from biomass (Lim et al., 2019; Linger et al., 2014; Yaegashi et al., 2017). However, often, the catabolic activities of wildtype microorganisms are not high enough, and further engineering is required. While there are many successful studies that rationally engineered to improve the utilization of native or non-native carbon sources (Antonovsky et al., 2016; Enquist-Newman et al., 2014; Lim et al., 2013; Wargacki et al., 2012), an initial design could fail or result in unsatisfactory utilization due to insufficient knowledge or understanding to precisely engineer a microorganism. In this regard, our study clearly demonstrated that an ALE strategy can complement a rational strain design strategy and generate improved strains by efficiently seeking beneficial mutations from a large sequence space. In addition to the aspect of the strain generation, we also showed that ALE allows a deeper understanding of host microorganisms by performing multi-scale analyses of evolved and reverse engineered strains. The genome and transcriptome sequencing of independently evolved clones were crucial for understanding how xylose and galactose are catabolized and to identify rate-limiting steps in KT2440. In addition, the analysis was also important to understand the range of phenotypes possible in evolved clones and utilized to further engineer the strains.

Interestingly, the final growth rates of P. putida xylD evolved clones on xylose (0.23-0.25 h⁻¹) were relatively smaller than those of P. putida galETKM on galactose (0.35-0.52 h⁻¹). Different endpoint growth rates depending on sugars were also observed in a previous ALE study with E. coli K-12 MG1655 (Sandberg et al., 2017). Although it is not currently clear why the growth rates on xylose remained at low level and were not further improved, considering that typical biomass hydrolysates contain multiple sugars, the P. putida xylD clones could consume other sugars (e.g., glucose, FIG. 8) and show higher growth rates during fermentation with actual biomass-derived feedstocks. If even higher growth rates are desired, further ALE experiments with increased mutation rates or combining multiple catabolic pathways could be performed. Growth rates on glucose greater than 0.8 h⁻¹ have been observed in previous ALE studies (Lim et al., 2020; Mohamed et al., 2020). Finally, the finding that similar growth rates of the evolved clones on glucose to the wild type (FIG. 8) indicates that the evolved strains are not entirely specialized and the mutations they possess appear to be local to the targeted sugar uptake pathways.

Additionally, more value can be added if the simultaneous utilization of multiple sugars is studied and ALE can similarly aide in the optimization of such strains. A bioprocess for the simultaneous utilization can be directly designed by co-culturing the generated strains for the specified utilization of sugars (L. Wang et al., 2019; Zhang et al., 2015). More promisingly, separately evolved and optimized pathways and mutations could be introduced into a single strain and inherent preferential utilization mechanisms can be deregulated. Previously, the deletion of carbon catabolite repression protein (Crc) was shown to enable the simultaneous utilization of sugars and aromatics (Elmore et al., 2020). Alternatively, one can also apply another ALE strategy that is growing a strain under a substrate-switching condition or mixed-substrate condition (Quan et al., 2012; Sandberg et al., 2017) to facilitate the utilization of multiple sugars. These efforts have the potentially to greatly improve titers, productivities, and yields, by increasing the substrate consumption rate (i.e., front end engineering), which are critical measures in bioprocess.

In summary, we successfully generated P. putida KT2440 strains for the efficient utilization of xylose and galactose. The ALE approach successfully overcomes the limitation of rational strain designing and enabled significantly improved xylose and galactose utilization capabilities. Furthermore, our indigoidine production results promise the strong potential of the developed strains to improve the economic viability. We believe the developed strains as well as mutational mechanisms could be useful to develop efficient biomass-converting processes.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A Pseudomonas cell is able to grow in a medium with xylose or galactose as a sole carbon source with a growth rate of equal to or higher than 0.10 h⁻¹.

2. The Pseudomonas cell of claim 1, wherein the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P, oryzihabitans, P. parafluva, or P. plecoglossicida.

3. The Pseudomonas cell of claim 2, wherein the Pseudomonas cell is a P. putida cell.

4. The Pseudomonas cell of claim 3, wherein the P. putida cell is strain KT2440.

5. The Pseudomonas cell of claim 1, wherein the Pseudomonas cell comprises the following genes: (A) a gene encoding a heterologous xylonate dehydratase, and mutations in native ptxS and/or kguT genes encoding a 2-ketogluconate operon repressor and 2-ketogluconate transporter, respectively; and/or, (B) genes encoding heterologous UDP-glucose 4-epimerase, galactose-1-phosphate uridylyltransferase, galactokinase, and galactose-1-epimerase, and mutation(s) in a native gtsABCD gene encoding an ATP-binding cassette (ABC) sugar transporting system.

6. The Pseudomonas cell of claim 5, wherein the heterologous xylonate dehydratase is Caulobacter crescentus xylD gene.

7. The Pseudomonas cell of claim 5, wherein the genes encoding heterologous UDP-glucose 4-epimerase, galactose-1-phosphate uridylyltransferase, galactokinase, and galactose-l-epimerase are Escherichia coli galETKM genes.

8. The Pseudomonas cell of claim 5, wherein the Pseudomonas cell is capable of utilizing xylose as a sole carbon source, and the Pseudomonas cell comprises the following genes: a gene encoding xylonate dehydratase, and mutations in ptxS and/or kguT genes encoding a 2-ketogluconate operon repressor and 2-ketogluconate transporter, respectively.

9. The Pseudomonas cell of claim 8, wherein the gene encoding xylonate dehydratase is Caulobacter crescentus xylD gene.

10. The Pseudomonas cell of claim 8, wherein the Pseudomonas cell growing in a medium with xylose as a sole carbon source has a growth rate of equal to or higher than 0.10 h⁻¹, 0.15 h⁻¹, 0.20 h⁻¹, or 0.25 h⁻¹.

11. The Pseudomonas cell of claim 5, wherein the Pseudomonas cell capable of utilizing galactose as a sole carbon source, and the Pseudomonas cell comprises the following genes: genes encoding UDP-glucose 4-epimerase, galactose-l-phosphate uridylyltransferase, galactokinase, and galactose-1-epimerase, and mutation(s) in gtsABCD genes encoding an ATP-binding cassette (ABC) sugar transporting system.

12. The Pseudomonas cell of claim 11, wherein the genes encoding UDP-glucose 4-epimerase, galactose-l-phosphate uridylyltransferase, galactokinase, and galactose-1-epimerase are Escherichia coli galETKM genes.

13. The Pseudomonas cell of claim 11, wherein the Pseudomonas cell growing in a medium with galactose as a sole carbon source has a growth rate of equal to or higher than 0.10 h⁻¹, 0.15 h⁻¹, 0.20 h⁻¹, 0.25 h⁻¹, 0.30 h⁻¹, 0.35 h⁻¹, 0.40 h⁻¹, 0.45 h⁻¹, 0.50 h⁻¹, or 0.52 h⁻¹.

14. The Pseudomonas cell of claim 5, wherein the native ptxS, kguT, and/or gtsABCD genes are knocked out or deleted from the chromosome of the Pseudomonas cell.

15. The Pseudomonas cell of claim 5, wherein the xylD gene and/or the galETKM genes are each independently capable of expression from the chromosome under a constitutive promoter and 5′-untranslated region.
